(19) United States
(12) Patent Application Publication
(10) Pub. No.: US 2007/0199090 A1
(43) Pub. Date: Aug. 23, 2007

(54) Modulating alkaloid biosynthesis

(76) Inventors: Nestor Apuya, Culver City, CA (US); Steven Craig Bobzin, Malibu, CA (US); Joon-Hyun Park, Oak Park, CA (US)

Correspondence Address: FISH & RICHARDSON P.C., P.O. BOX 1022, MINNEAPOLIS, MN, 55440-1022, US

(21) Appl. No.: 11/360,039

(22) Filed: Feb. 22, 2006

(51) Int. Cl. A01H 11/00; C12N 9/10; C12N 15/82; C12N 5/04; A01H 1/00

(52) U.S. Cl. 800/278; 435/419; 435/468; 435/193; 800/295

(57) ABSTRACT

Materials and methods for modulating expression of nucleic acid sequences of interest, e.g., nucleic acid sequences involved in alkaloid biosynthesis, are disclosed. For example, plants and plant cells containing a regulatory protein that can modulate expression of a gene(s), such as an alkaloid biosynthesis gene(s), are disclosed.

### TECHNICAL FIELD

This document relates to materials and methods involved in modulating gene expression in plants. For example, this document relates to materials and methods for using a regulatory protein to modulate the expression of nucleic acid sequences of interest, such as those involved in alkaloid biosynthesis.

### INCORPORATION-BY-REFERENCE & TEXTS

The material on the accompanying diskette is hereby incorporated by reference into this application. The accompanying compact discs contain one file, 11696-157002..txt, which was created on Feb. 22, 2006. The file named 11696-157002..txt is 232 KB. The file can be accessed using Microsoft Word on a computer that uses Windows OS.

### BACKGROUND

Plant families that produce alkaloids include the Papaveraceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, and Rutaceae families. Many alkaloids isolated from such plants are known for their pharmacologic (e.g., narcotic), insecticidal, and physiologic effects. For example, the poppy (Papaveraceae) family contains about 250 species found mainly in the northern temperate regions of the world. The principal morphinan alkaloids in opium poppy (Papaver somniferum) are morphine, codeine, and thebaine, which are used directly or modified using synthetic methods to produce pharmaceutical compounds used for pain management, cough suppression, and addiction.

### SUMMARY

The present invention relates to materials and methods for modulating expression of nucleic acid sequences, such as those encoding polypeptides involved in biosynthesis of alkaloids. For example, the invention relates to regulatory proteins identified as being capable of modulating expression of polypeptides involved in alkaloid biosynthesis. Modulation of expression can include up-regulation or activation, e.g., an increase of expression relative to basal or native states (e.g., a control level). In other cases, modulation of expression can include down-regulation or repression, e.g., a decrease of expression relative to basal or native states, such as the level in a control. Such regulatory proteins can be used to create transgenic plants, e.g., plants capable of producing one or more alkaloids. Such plants can have modulated, e.g., increased, amounts and/or rates of biosynthesis of one or more alkaloid compounds. Regulatory proteins can also be used along with their cognate promoters to modulate expression of one or more endogenous sequences, e.g., alkaloid biosynthesis genes, in a plant cell. Given the variety of uses of the various alkaloid classes of compounds, it would be useful to control selective expression of one or more proteins, including enzymes, regulatory proteins, and other auxiliary proteins, involved in alkaloid biosynthesis, e.g., to regulate biosynthesis of known and/or novel alkaloids.

In one aspect, a plant cell is provided. The plant cell comprises an exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, where the nucleic acid is operably linked to a regulatory region that modulates transcription of the regulatory protein in the plant cell.

The regulatory region can be a promoter. The promoter can be a tissue-preferential promoter. The tissue can be stem, seed pod, parenchymal, or reproductive tissue. The promoter can be a cell type-preferential promoter. The cell type can be a laticifer cell, a companion cell, or a sieve element cell. The promoter can be an inducible promoter.

The plant cell further can comprise an endogenous regulatory region that is associated with the regulatory protein or an exogenous regulatory region operably linked to a sequence of interest. The exogenous regulatory region, which is associated with the regulatory protein, comprises a nucleic acid having 80% or greater sequence identity to a regulatory region set forth in SEQ ID NOs:22-37.

The plant cell can be capable of producing one or more alkaloids. At least one of the one or more alkaloids can be a morphinan alkaloid, a morphinan analog alkaloid, a tetrahydrobenzylisoquinoline alkaloid, or a benzophenanthridine alkaloid. At least one of the one or more alkaloids can be a monoterpenoid indole alkaloid, a bisbenzylisoquinoline alkaloid, a pyridine, purine, tropane, or quinoline alkaloid, a terpenoid, betaine, or phenethylamine alkaloid, or a steroid alkaloid.

The plant cell can be a member of the Papaveraceae, Menispermaceae, Lauraceae, Euphorbiaceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, or Rutaceae families. The plant cell can be a member of the species Papaver bracteatum, Papaver orientale, Papaver setigerum, Papaver somniferum, Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, or Beilschmiedia oreophila.

The plant cell further can comprise a nucleic acid encoding a second regulatory protein operably linked to a second regulatory region that modulates transcription of the second regulatory protein in the plant cell. The nucleic acid encoding a second regulatory protein operably linked to a second regulatory region can be present on a second recombinant nucleic acid construct.

The sequence of interest can comprise a coding sequence for a polypeptide involved in alkaloid biosynthesis. The polypeptide can be a regulatory protein involved in alkaloid biosynthesis. The polypeptide can be an alkaloid biosynthesis enzyme.

The enzyme can be a morphinan alkaloid biosynthesis enzyme, a tetrahydrobenzylisoquinoline alkaloid biosynthesis enzyme, or a benzophenanthridine alkaloid biosynthesis enzyme.

The enzyme can be a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

The enzyme can be selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

The enzyme can be selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

The enzyme can be selected from the group consisting of dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

The regulatory protein-regulatory region association can be effective for modulating the amount of at least one alkaloid compound in the cell. The at least one alkaloid compound can be selected from the group consisting of salutaridine, salutaridinol, salutaridinol acetate, thebaine, isothebaine, papaverine, narcotine, noscapine, narceine, hydrastine, oripavine, morphinone, morphine, codeine, codeinone, and neopinone. The at least one alkaloid compound can be selected from the group consisting of berberine, palmatine, tetrahydropalmatine, S-canadine, columbamine, S-tetrahydrocolumbamine, S-scoulerine, S-cheilathifoline, S-stylopine, S-cis-N-methylstylopine, protopine, 6-hydroxyprotopine, R-norreticuline, S-norreticuline, R-reticuline, S-reticuline, 1,2-dehydroreticuline, S-3′-hydroxycoclaurine, S-norcoclaurine, S-coclaurine, S-N-methylcoclaurine, berbamunine, 2′-norberbamunine, and guatteguamerine. The at least one alkaloid compound can be selected from the group consisting of dihydro-sanguinarine, sanguinarine, dihydroxy-dihydro-sanguinarine, 12-hydroxy-dihydrochelirubine, 10-hydroxy-dihydro-sanguinarine, dihydro-macarpine, dihydro-chelirubine, dihydro-sanguinarine, chelirubine, 12-hydroxy-chelirubine, and macarpine.

In another aspect, a Papaveraceae plant is provided. The Papaveraceae plant comprises an exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, where the nucleic acid is operably linked to a regulatory region that modulates transcription of the regulatory protein in the plant cell.

The regulatory protein can modulate expression of an endogenous polypeptide involved in alkaloid biosynthesis in the cell. The endogenous polypeptide can be a regulatory protein involved in alkaloid biosynthesis. The endogenous polypeptide can be an alkaloid biosynthesis enzyme.

The enzyme can be a morphinan alkaloid biosynthesis enzyme, a tetrahydrobenzylisoquinoline alkaloid biosynthesis enzyme, or a benzophenanthridine alkaloid biosynthesis enzyme.

The enzyme can be a monoterpenoid indole alkaloid biosynthesis enzyme, a bisbenzylisoquinoline alkaloid biosynthesis enzyme, a pyridine, purine, tropane, or quinoline alkaloid biosynthesis enzyme, a terpenoid, betaine, or phenethylamine alkaloid biosynthesis enzyme, or a steroid alkaloid biosynthesis enzyme.

The enzyme can be selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247).

The enzyme can be selected from the group consisting of tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH; EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), and columbamine oxidase (EC 1.21.3.2).

The enzyme can be selected from the group consisting of dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase (EC 1.14.13.57), and 12-hydroxydihydrochelirubine 12-O-methyltransferase (EC 2.1.1.120).

In another aspect, a method of modulating the expression level of one or more genes in a plant cell is provided. The method comprises transforming the plant cell with an isolated nucleic acid comprising a nucleotide sequence encoding a regulatory protein comprising a polypeptide sequence set forth in SEQ ID NOs:2-13, SEQ ID NOs:15-19, SEQ ID NO:21, or a consensus sequence set forth in FIG. 1 or FIG. 2, where the nucleotide sequence is operably linked to a regulatory region that modulates transcription in the plant cell. The plant cell can be a member of the Papaveraceae family. The one or more genes can be involved in alkaloid biosynthesis.

In another aspect, a method of expressing a sequence of interest is provided. The method comprises growing a plant cell comprising (1) an exogenous nucleic acid comprising a regulatory region comprising a nucleic acid having 80% or greater sequence identity to a regulatory region set forth in SEQ ID NOs:22-37, where the regulatory region is operably linked to a sequence of interest; and (2) an exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2. The regulatory region and the regulatory protein are associated. The plant cell is grown under conditions effective for the expression of the regulatory protein.

In another aspect, a method of expressing an endogenous sequence of interest is provided. The method comprises growing a plant cell comprising an endogenous regulatory region operably linked to a sequence of interest. The endogenous regulatory region comprises a nucleic acid having 80% or greater sequence identity to a regulatory region set forth in SEQ ID NOs:22-37. The plant cell further comprises a nucleic acid encoding an exogenous regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2. The exogenous regulatory protein and the endogenous regulatory region are associated. The plant cell is grown under conditions effective for the expression of the exogenous regulatory protein. The sequence of interest can comprise a coding sequence for a polypeptide involved in alkaloid biosynthesis.

In another aspect, a method of expressing an exogenous sequence of interest is provided. The method comprises growing a plant cell comprising an exogenous regulatory region operably linked to a sequence of interest. The exogenous regulatory region comprises a nucleic acid having 80% or greater sequence identity to a regulatory region set forth in SEQ ID NOs:22-37. The plant cell further comprises a nucleic acid encoding an endogenous regulatory protein. The endogenous regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2. The regulatory region and the regulatory protein are associated. The plant cell is grown under conditions effective for the expression of the endogenous regulatory protein. The sequence of interest can comprise a coding sequence for a polypeptide involved in alkaloid biosynthesis.

In another aspect, a method of producing one or more alkaloids in a plant cell is provided. The method comprises growing a plant cell comprising an exogenous nucleic acid. The exogenous nucleic acid comprises a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2. The nucleic acid is operably linked to a regulatory region that modulates transcription of the regulatory protein in the plant cell. The plant cell further comprises an endogenous regulatory region that is associated with the regulatory protein. The endogenous regulatory region is operably linked to a sequence of interest comprising a coding sequence for a polypeptide involved in alkaloid biosynthesis. The plant cell is capable of producing one or more alkaloids. The plant cell is grown under conditions effective for the expression of the regulatory protein.

In another aspect, a method of producing one or more alkaloids in a plant cell is provided. The method comprises growing a plant cell comprising an exogenous nucleic acid. The exogenous nucleic acid comprises a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2. The nucleic acid is operably linked to a regulatory region that modulates transcription of the regulatory protein in the plant cell. The plant cell further comprises an exogenous regulatory region operably linked to a sequence of interest. The exogenous regulatory region is associated with the regulatory protein. The exogenous regulatory region comprises a nucleic acid having 80% or greater sequence identity to a regulatory region set forth in SEQ ID NOs:22-37. The sequence of interest comprises a coding sequence for a polypeptide involved in alkaloid biosynthesis. The plant cell is grown under conditions effective for the expression of the regulatory protein.

In another aspect, a method of modulating the level of one or more alkaloid compounds in a plant cell is provided. The method comprises transforming the plant cell with an isolated nucleic acid comprising a nucleotide sequence encoding a regulatory protein comprising a polypeptide sequence set forth in SEQ ID NOs:2-13, SEQ ID NOs:15-19, SEQ ID NO:21, or a consensus sequence set forth in FIG. 1 or FIG. 2. The nucleotide sequence is operably linked to a regulatory region that modulates transcription in the plant cell. A plant produced from the plant cell has a difference in the level of the alkaloid compared to the level of the alkaloid in a corresponding control plant that does not comprise the isolated nucleic acid. The plant cell can be a Papaveraceae family member.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is an alignment of the amino acid sequence of Lead cDNA ID 23461192 (5110B8; SEQ ID NO:2) with homologous and/or orthologous amino acid sequences gi|56384438 (SEQ ID NO:4), gi|9857294 (SEQ ID NO:5), gi|5360186 (SEQ ID NO:6), gi|38112202 (SEQ ID NO:7), gi|1370274 (SEQ ID NO:8), gi|17402597 (SEQ ID NO:9), gi|1673406 (SEQ ID NO:10), CeresClone:921919 (SEQ ID NO:11), gi|1772985 (SEQ ID NO:12), and gi|50900462 (SEQ ID NO:13). The consensus sequence determined by the alignment is set forth.

FIG. 2 is an alignment of the amino acid sequence of Lead cDNA ID 23660631 (5110C9; SEQ ID NO:15) with homologous and/or orthologous amino acid sequences CeresClone:763471 (SEQ ID NO:16), CeresClone:218529 (SEQ ID NO:17), and CeresClone:48 1192 (SEQ ID NO:18). The consensus sequence determined by the alignment is set forth.

### DETAILED DESCRIPTION

The invention is based, in part, on the discovery of regulatory proteins that can modulate expression of a reporter polypeptide operably linked to a regulatory region, such as a regulatory region involved in alkaloid biosynthesis. A regulatory protein and a regulatory region are considered to be associated when the regulatory protein is capable of modulating expression of a nucleic acid operably linked to the regulatory region. A regulatory protein and its associated regulatory region can be used to selectively modulate expression of a sequence of interest, when such a sequence is operably linked to the regulatory region. In addition, the use of such regulatory protein-regulatory region associations in plants can permit selective modulation of the amount or rate of biosynthesis of plant polypeptides and plant compounds, such as alkaloid compounds, under a desired environmental condition or in a desired plant developmental pathway. For example, the use of recombinant regulatory proteins in plants, such as Papaveraceae plants, that are capable of producing one or more alkaloids, can permit selective modulation of the amount of such compounds in such plants.

Polypeptides

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

The term “isolated” with respect to a polypeptide refers to a polypeptide that has been separated from cellular components that naturally accompany it. Typically, the polypeptide is isolated when it is at least 60%, e.g., 70%, 80%, 90%, 95%, or 99%, by weight, free from proteins and naturally occurring organic molecules that are naturally associated with it. In general, an isolated polypeptide will yield a single major band on a reducing and/or non-reducing polyacrylamide gel. Isolated polypeptides can be obtained, for example, by extraction from a natural source (e.g., plant tissue), chemical synthesis, or by recombinant production in a host plant cell. To recombinantly produce a polypeptide, a nucleic acid sequence containing a nucleotide sequence encoding a polypeptide of interest can be ligated into an expression vector and used to transform a bacterial, eukaryotic, or plant host cell, e.g., insect, yeast, mammalian, or plant cells.

Polypeptides described herein include regulatory proteins. Such a regulatory protein typically is effective for modulating expression of a nucleic acid sequence operably linked to a regulatory region involved in an alkaloid biosynthesis pathway, such as a nucleic acid sequence encoding a polypeptide involved in alkaloid biosynthesis. Modulation of expression of a nucleic acid sequence can be either an increase or a decrease in expression of the nucleic acid sequence relative to the average rate or level of expression of the nucleic acid sequence in a control plant.

A regulatory protein can contain an FHA domain. A forkhead-associated (FHA) domain is a phosphopeptide recognition domain found in many regulatory proteins. It displays specificity for phosphothreonine-containing epitopes but will also recognize phosphotyrosine with relatively high affinity. An FHA domain spans approximately 80-100 amino acid residues folded into an 11-stranded beta sandwich, which sometimes contain small helical insertions between the loops connecting the strands. The FHA domain is present in a diverse range of proteins, such as kinases, phosphatases, kinesins, transcription factors, RNA-binding proteins and metabolic enzymes which partake in many different cellular processes, such as DNA repair, signal transduction, vesicular transport and protein degradation.

A regulatory protein containing an FHA domain can also contain Pyr_redox and Pyr_redox_{—}2 domains, both of which are characteristic of polypeptides belonging to the pyridine nucleotide-disulphide oxidoreductase family. This family includes class I and class II oxidoreductases and also NADH oxidases and peroxidases. Pyr_redox and Pyr_redox_{—}2 domains are each annotated as a small NADH binding domain within a larger FAD binding domain. An FAD binding domain, such as an FAD_binding_{—}3 domain, is involved in FAD binding in a number of enzymes.

SEQ ID NO:2 sets forth the amino acid sequence of a DNA clone, identified herein as cDNA ID 23461192 (SEQ ID NO:1) that is predicted to encode a polypeptide containing a FHA, Pyr_redox, Pyr_redox_{—}2, and an FAD_binding_{—}3 domain. A regulatory protein can comprise the amino acid sequence set forth in SEQ ID NO:2. Alternatively, a regulatory protein can be a homolog, ortholog, or variant of the polypeptide having the amino acid sequence set forth in SEQ ID NO:2. For example, a regulatory protein can comprise an amino acid sequence with at least 60% sequence identity, e.g., 60%, 61%, 62%, 63%, 64%, 65%, 67%, 68%, 70%, 75%, 80%, 85%, 90%, 95%, 97%, 98%, or 99% sequence identity, to the amino acid sequence set forth in SEQ ID NO:2.

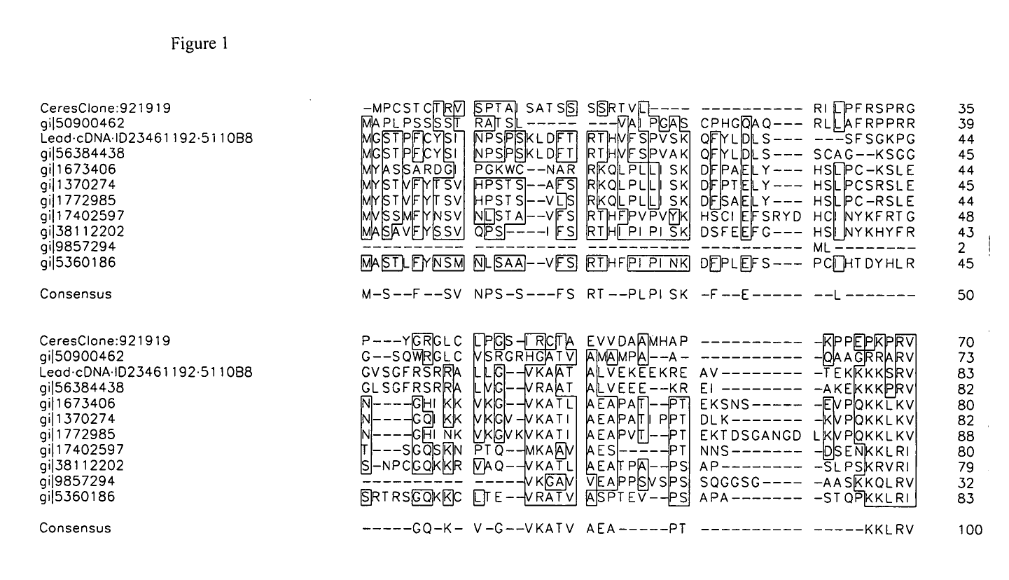

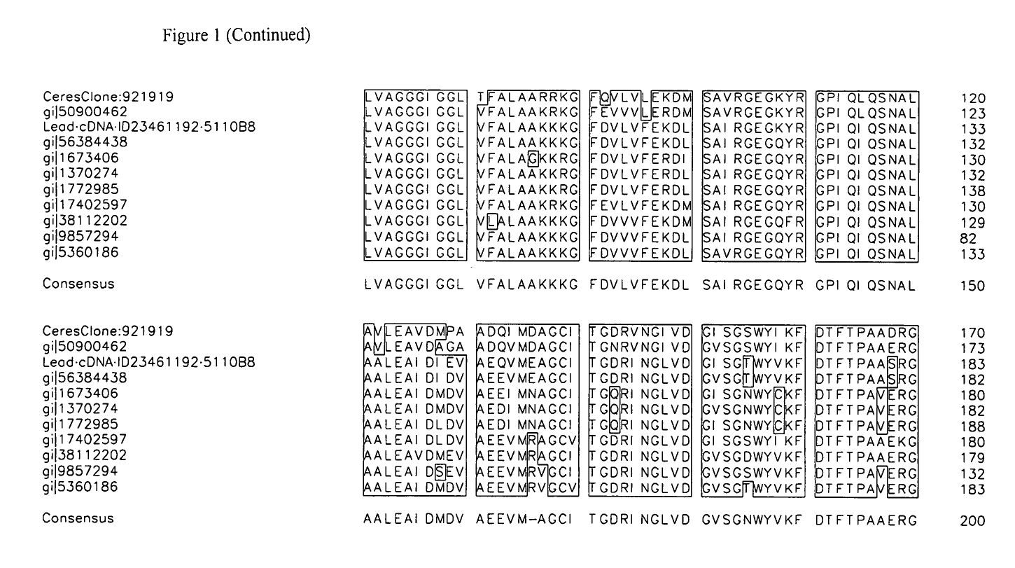

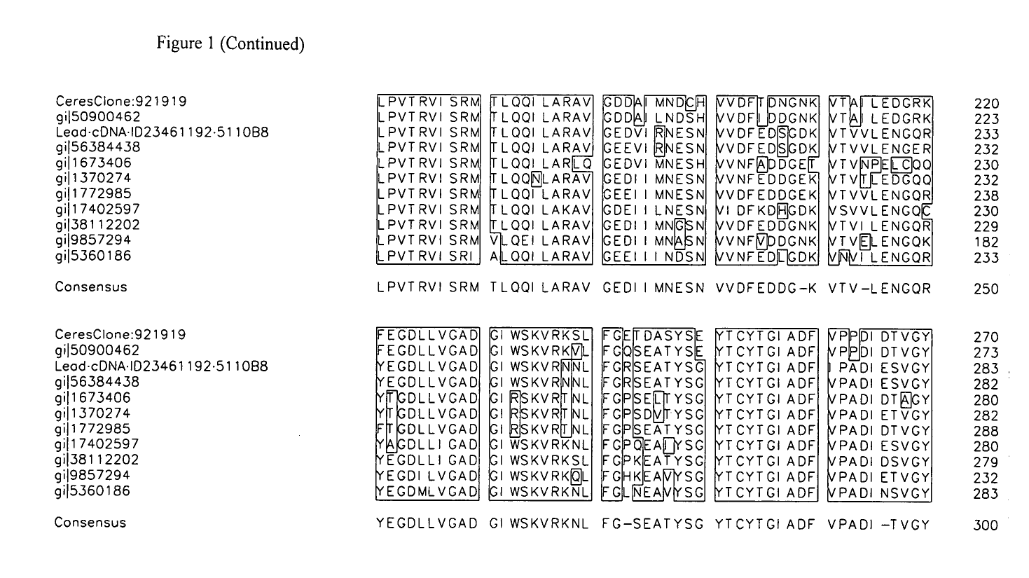

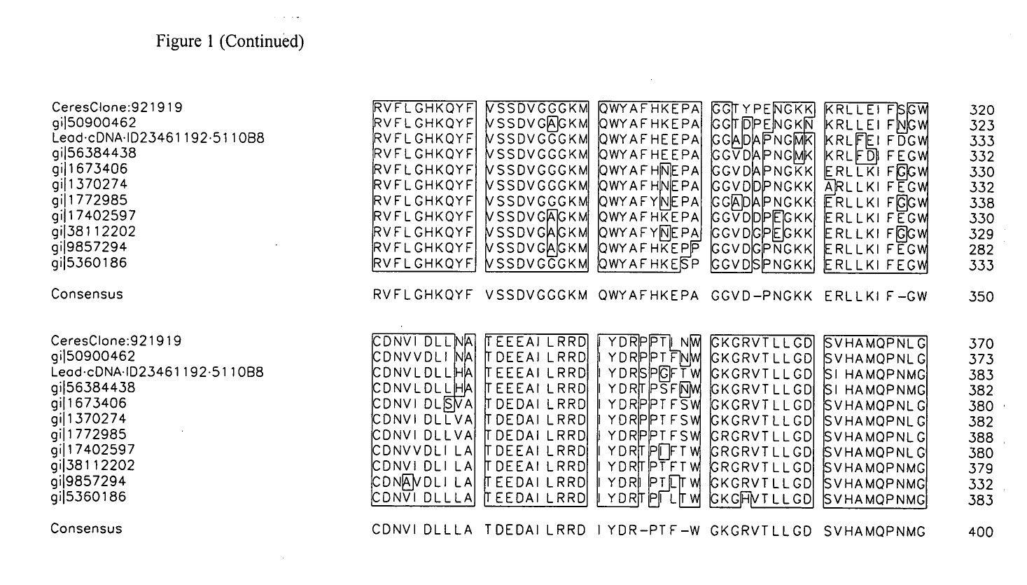

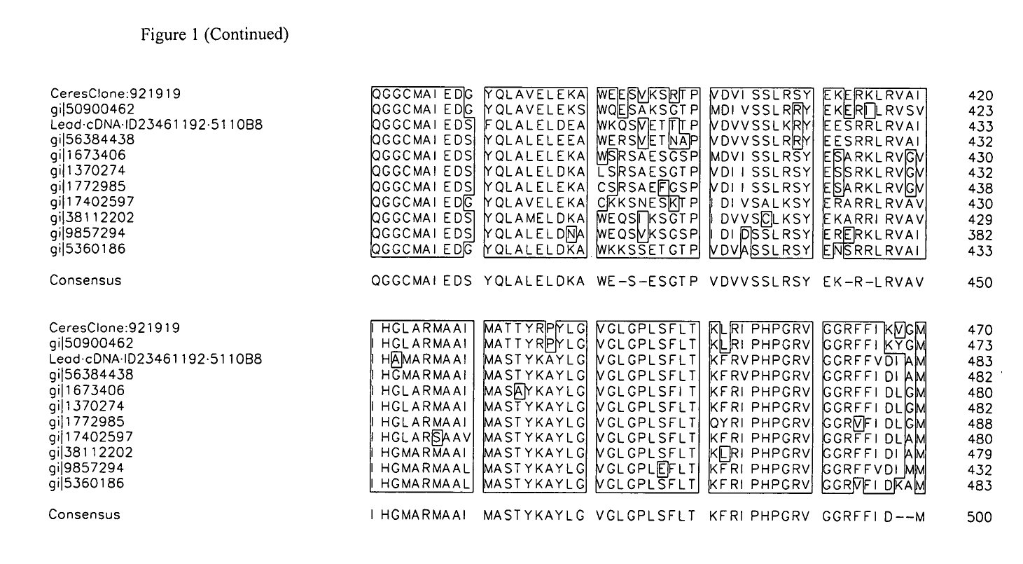

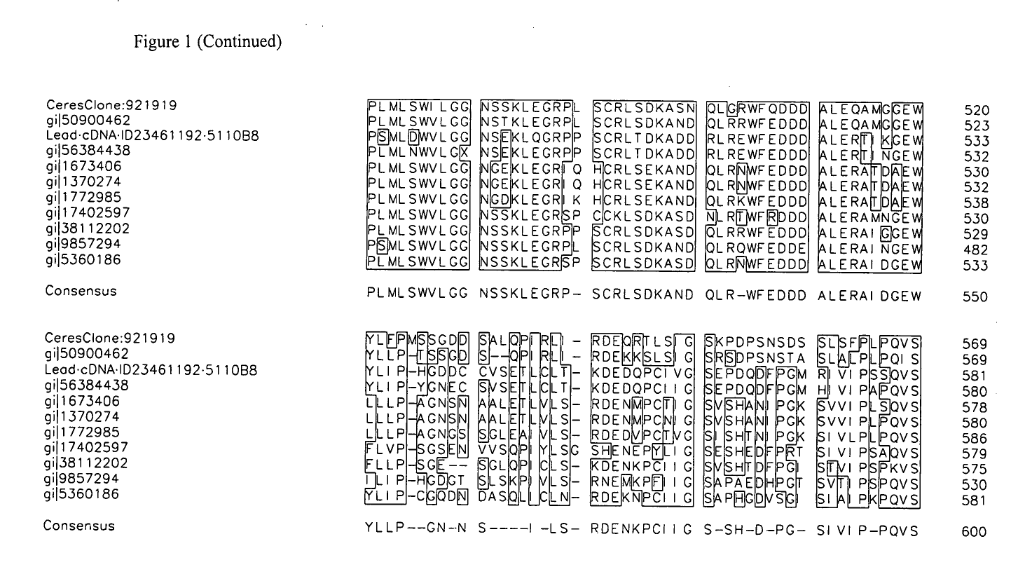

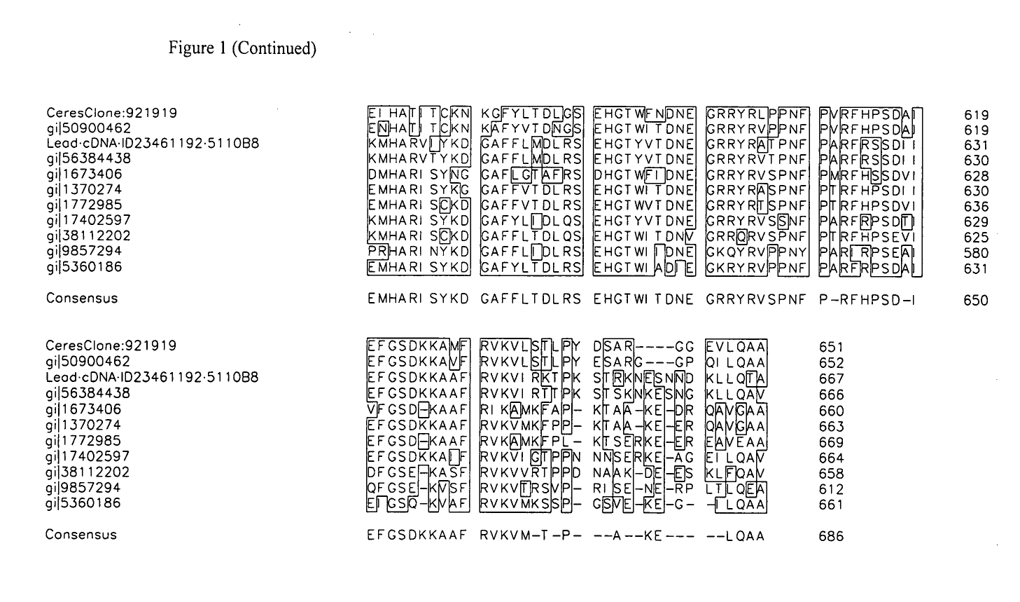

Amino acid sequences of homologs and/or orthologs of the polypeptide having the amino acid sequence set forth in SEQ ID NO:2 are provided in FIG. 1. FIG. 1 also includes a consensus amino acid sequence determined by aligning homologous and/or orthologous amino acid sequences with the amino acid sequence set forth in SEQ ID NO:2. For example, the alignment in FIG. 1 provides the amino acid sequences of cDNA ID 23461192 (SEQ ID NO:2), gi|56384438 (SEQ ID NO:4), gi|9857294 (SEQ ID NO:5), gi|5360186 (SEQ ID NO:6), gi|38112202 (SEQ ID NO:7), gi|1370274 (SEQ ID NO:8), gi|17402597 (SEQ ID NO:9), gi|1673406 (SEQ ID NO: 10), CeresClone:921919 (SEQ ID NO:11), gi|1772985 (SEQ ID NO: 12), and gi|50900462 (SEQ ID NO: 13). Other homologs and/or orthologs of SEQ ID NO:2 include gi|11602842 (SEQ ID NO:3).

In some cases, a regulatory protein can comprise a polypeptide having at least 80% sequence identity, e.g., 80%, 85%, 90%, 93%, 95%, 97%, 98%, or 99% sequence identity, to an amino acid sequence corresponding to gi|11602842 (SEQ ID NO:3), gi|56384438 (SEQ ID NO:4), gi|9857294 (SEQ ID NO:5), gi|5360186 (SEQ ID NO:6), gi|38112202 (SEQ ID NO:7), gi|1370274 (SEQ ID NO:8), gi|17402597 (SEQ ID NO:9), gi|1673406 (SEQ ID NO: 10), CeresClone:921919 (SEQ ID NO:11), gi|1772985 (SEQ ID NO: 12), gi|50900462 (SEQ ID NO: 13) or the consensus sequence set forth in FIG. 1.

A regulatory protein can have an ACT domain characteristic of polypeptides having a regulatory role. ACT domains are linked to a wide range of metabolic enzymes that are regulated by amino acid concentration. Pairs of ACT domains bind specifically to a particular amino acid leading to regulation of the linked enzyme. The ACT domain is found in a variety of contexts and is proposed to be a conserved regulatory binding fold. ACT domains are linked to a wide range of metabolic enzymes that are regulated by amino acid concentration. The archetypical ACT domain is the C-terminal regulatory domain of 3-phosphoglycerate dehydrogenase (3PGDH), which folds with a ferredoxin-like topology. A pair of ACT domains form an eight-stranded antiparallel sheet with two molecules of allosteric inhibitor serine bound in the interface.

SEQ ID NO:15 sets forth the amino acid sequence of a DNA clone, identified herein as cDNA ID 23660631 (SEQ ID NO:14) that is predicted to encode a polypeptide containing an ACT domain. A regulatory protein can comprise the amino acid sequence set forth in SEQ ID NO:15. Alternatively, a regulatory protein can be a homolog, ortholog, or variant of the polypeptide having the amino acid sequence set forth in SEQ ID NO:15. For example, a regulatory protein can comprise an amino acid sequence with at least 65% sequence identity, e.g., 65%, 67%, 68%, 70%, 75%, 80%, 85%, 90%, 95%, 97%, 98%, or 99% sequence identity, to the amino acid sequence set forth in SEQ ID NO:15.

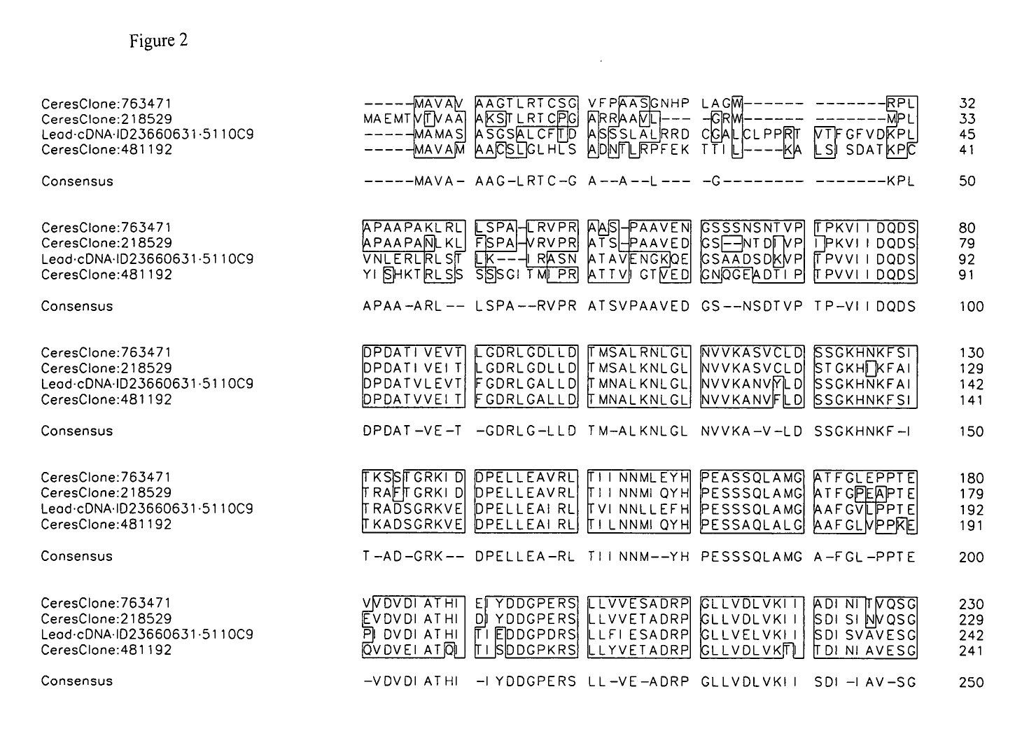

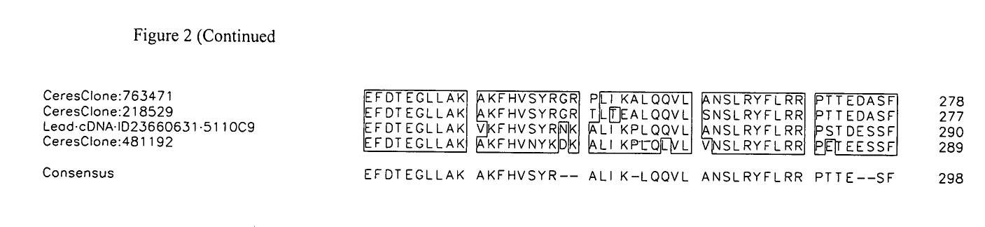

Amino acid sequences of homologs and/or orthologs of the polypeptide having the amino acid sequence set forth in SEQ ID NO:15 are provided in FIG. 2. FIG. 2 also includes a consensus amino acid sequence determined by aligning homologous and/or orthologous amino acid sequences with the amino acid sequence set forth in SEQ ID NO:15. For example, the alignment in FIG. 2 provides the amino acid sequences of cDNA ID 23660631 (SEQ ID NO: 15), CeresClone:763471 (SEQ ID NO:16), CeresClone:218529 (SEQ ID NO:17), and CeresClone:481192 (SEQ ID NO:18). Other homologs and/or orthologs of SEQ ID NO:15 include CeresClone:517528 (SEQ ID NO:19).

In some cases, a regulatory protein can comprise a polypeptide having at least 80% sequence identity, e.g., 80%, 85%, 90%, 93%, 95%, 97%, 98%, or 99% sequence identity, to an amino acid sequence corresponding to CeresClone:763471 (SEQ ID NO:16), CeresClone:218529 (SEQ ID NO:17), CeresClone:481192 (SEQ ID NO:18), CeresClone:517528 (SEQ ID NO: 19), or the consensus sequence set forth in FIG. 2.

A regulatory protein can have a Glyco_hydro_{—}2_N sugar binding domain characteristic of polypeptides belonging to glycosyl hydrolases family 2. O-Glycosyl hydrolases are a widespread group of enzymes that hydrolyze the glycosidic bond between two or more carbohydrates, or between a carbohydrate and a non-carbohydrate moiety. Glycoside hydrolase family 2 comprises enzymes with known activities, such as beta-galactosidase, beta-mannosidase and beta-glucuronidase activities. These enzymes contain a conserved glutamic acid residue which has been shown, in Escherichia coli lacZ, to be the general acid/base catalyst in the active site of the enzyme. The sugar binding domain has a jelly-roll fold.

A regulatory protein containing a Glyco_hydro_{—}2_N domain can also contain a myosin tail. Tail domains of myosin II heavy chains associate in a rod-like α-helical coiled coil. The coiled coil is composed of the tail from two molecules of myosin. These can then assemble into the macromolecular thick filament. The coiled-coil region provides the structural backbone of the thick filament.

SEQ ID NO:21 sets forth the amino acid sequence of a DNA clone, identified herein as cDNA ID 23777863 (SEQ ID NO:20) that is predicted to encode a polypeptide containing Glyco_hydro_{—}2_N domain. A regulatory protein can comprise the amino acid sequence set forth in SEQ ID NO:21. Alternatively, a regulatory protein can be a homolog, ortholog, or variant of the polypeptide having the amino acid sequence set forth in SEQ ID NO:21. For example, a regulatory protein can comprise an amino acid sequence with at least 40% sequence identity, e.g., 40%, 45%, 50%, 55%, 60%, 65%, 67%, 68%, 70%, 75%, 80%, 85%, 90%, 95%, 97%, 98%, or 99% sequence identity, to the amino acid sequence set forth in SEQ ID NO:21.

A regulatory protein encoded by a recombinant nucleic acid can be a native regulatory protein, i.e., one or more additional copies of the coding sequence for a regulatory protein that is naturally present in the cell. Alternatively, a regulatory protein can be heterologous to the cell, e.g., a transgenic Papaveraceae plant can contain the coding sequence for a regulatory protein from a Catharanthus plant.

A regulatory protein can include additional amino acids that are not involved in modulating gene expression, and thus can be longer than would otherwise be the case. For example, a regulatory protein can include an amino acid sequence that functions as a reporter. Such a regulatory protein can be a fusion protein in which a green fluorescent protein (GFP) polypeptide is fused to, e.g., SEQ ID NO:2, or in which a yellow fluorescent protein (YFP) polypeptide is fused to, e.g., SEQ ID NO:15. In some embodiments, a regulatory protein includes a purification tag, a chloroplast transit peptide, a mitochondrial transit peptide, or a leader sequence added to the amino or carboxy terminus.

Regulatory protein candidates suitable for use in the invention can be identified by analysis of nucleotide and polypeptide sequence alignments. For example, performing a query on a database of nucleotide or polypeptide sequences can identify homologs and/or orthologs of regulatory proteins. Sequence analysis can involve BLAST, Reciprocal BLAST, or PSI-BLAST analysis of nonredundant databases using known regulatory protein amino acid sequences. Those polypeptides in the database that have greater than 40% sequence identity can be identified as candidates for further evaluation for suitability as regulatory proteins. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in regulatory proteins, e.g., conserved functional domains.

The identification of conserved regions in a template or subject polypeptide can facilitate production of variants of regulatory proteins. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999).

Conserved regions also can be determined by aligning sequences of the same or related polypeptides from closely related species. Closely related species preferably are from the same family. In some embodiments, alignment of sequences from two different species is adequate. For example, sequences from Arabidopsis and Zea mays can be used to identify one or more conserved regions.

Typically, polypeptides that exhibit at least about 40% amino acid sequence identity are useful to identify conserved regions. Conserved regions of related polypeptides can exhibit at least 45% amino acid sequence identity, e.g., at least 50%, at least 60%, at least 70%, at least 80%, or at least 90% amino acid sequence identity. In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within regulatory proteins. These conserved regions can be useful in identifying functionally similar (orthologous) regulatory proteins.

In some instances, suitable regulatory proteins can be synthesized on the basis of consensus functional domains and/or conserved regions in polypeptides that are homologous regulatory proteins. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Representative homologs and/or orthologs of regulatory proteins are shown in FIGS. 1-2. Each Figure represents an alignment of the amino acid sequence of a regulatory protein with the amino acid sequences of corresponding homologs and/or orthologs. Amino acid sequences of regulatory proteins and their corresponding homologs and/or orthologs have been aligned to identify conserved amino acids and to determine consensus sequences that contain frequently occurring amino acid residues at particular positions in the aligned sequences, as shown in FIGS. 1-2. A dash in an aligned sequence represents a gap, i.e., a lack of an amino acid at that position. Identical amino acids or conserved amino acid substitutions among aligned sequences are identified by boxes.

Each consensus sequence is comprised of conserved regions. Each conserved region contains a sequence of contiguous amino acid residues. A dash in a consensus sequence indicates that the consensus sequence either lacks an amino acid at that position or includes an amino acid at that position. If an amino acid is present, the residue at that position corresponds to one found in any aligned sequence at that position.

Useful polypeptides can be constructed based on the consensus sequence in FIG. 1 or FIG. 2. Such a polypeptide includes the conserved regions in the selected consensus sequence, arranged in the order depicted in the Figure from amino-terminal end to carboxy-terminal end. Such a polypeptide may also include zero, one, or more than one amino acid in positions marked by dashes. When no amino acids are present at positions marked by dashes, the length of such a polypeptide is the sum of the amino acid residues in all conserved regions. When amino acids are present at all positions marked by dashes, such a polypeptide has a length that is the sum of the amino acid residues in all conserved regions and all dashes.

A conserved domain in certain cases may be 1) a localization domain, 2) an activation domain, 3) a repression domain, or 4) an oligomerization domain. Consensus domains and conserved regions can be identified by homologous polypeptide sequence analysis as described above. A regulatory protein can also be a fragment of a naturally occurring regulatory protein. The suitability of polypeptides for use as regulatory proteins can be evaluated by functional complementation studies.

Nucleic Acids

A nucleic acid can comprise a coding sequence that encodes any of the regulatory proteins as set forth in SEQ ID NOs:2-13, SEQ ID NOs:15-19, SEQ ID NO:21, and the consensus sequences set forth in FIGS. 1-2. In some cases, a recombinant nucleic acid construct can include a nucleic acid comprising less than the full-length coding sequence of a regulatory protein. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given regulatory protein can be modified such that optimal expression in a particular plant species is obtained, using appropriate codon bias tables for that species.

A nucleic acid also can comprise a nucleotide sequence corresponding to any of the regulatory regions as set forth in SEQ ID NOs:22-122. In some cases, a nucleic acid can comprise a nucleotide sequence corresponding to any of the regulatory regions as set forth in SEQ ID NOs:22-122 and a coding sequence that encodes any of the regulatory proteins as set forth in SEQ ID NOs:2-13, SEQ ID NOs:15-19, SEQ ID NO:21, and the consensus sequences set forth in FIGS. 1-2.

The terms “nucleic acid” and “polynucleotide” are used interchangeably herein, and refer both to RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

An isolated nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). Chenna et al., Nucleic Acids Res., 31(13):3497-500 (2003).

ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration. For example, a transgenic plant containing an exogenous nucleic acid can be the progeny of a cross between a stably transformed plant and a non-transgenic plant. Such progeny are considered to contain the exogenous nucleic acid.

Similarly, a regulatory protein can be endogenous or exogenous to a particular plant or plant cell. Exogenous regulatory proteins, therefore, can include proteins that are native to a plant or plant cell, but that are expressed in a plant cell via a recombinant nucleic acid construct, e.g., a California poppy plant transformed with a recombinant nucleic acid construct encoding a California poppy regulatory protein.

Likewise, a regulatory region can be exogenous or endogenous to a plant or plant cell. An exogenous regulatory region is a regulatory region that is part of a recombinant nucleic acid construct, or is not in its natural environment. For example, a Nicotiana promoter present on a recombinant nucleic acid construct is an exogenous regulatory region when a Nicotiana plant cell is transformed with the construct.

A transgenic plant or plant cell in which the expression of one or more sequences of interest is modulated includes at least one recombinant nucleic acid construct, e.g., a nucleic acid construct comprising a nucleic acid encoding a regulatory protein or a nucleic acid construct comprising a regulatory region as described herein. In certain cases, more than one recombinant nucleic acid construct can be included (e.g., two, three, four, five, six, or more recombinant nucleic acid constructs). For example, two recombinant nucleic acid constructs can be included, where one construct includes a nucleic acid encoding one regulatory protein, and another construct includes a nucleic acid encoding a second regulatory protein. Alternatively, one construct can include a nucleic acid encoding one regulatory protein, while another includes a regulatory region. In other cases, a plant cell can include a recombinant nucleic acid construct comprising a nucleic acid encoding a regulatory protein and further comprising a regulatory region that associates with the regulatory protein. In such cases, additional recombinant nucleic acid constructs can also be included in the plant cell, e.g., containing additional regulatory proteins and/or regulatory regions.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a plant cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

As described herein, plant cells can be transformed with a recombinant nucleic acid construct to express a polypeptide of interest. The polypeptide can then be extracted and purified using techniques known to those having ordinary skill in the art.

Regulatory Regions

Particular regulatory regions were examined for their ability to associate with regulatory proteins described herein. The sequences of these regulatory regions are set forth in SEQ ID NOs:22-37. These regulatory regions were initially chosen for investigation because they were thought to be regulatory regions involved in alkaloid biosynthetic pathways in plants such as Arabidopsis, California poppy, and opium poppy. Using the methods described herein, regulatory proteins that can associate with some of these regulatory regions were identified, and such associations are listed in Table 4 (under Example 5 below). In turn, knowledge of a regulatory protein-regulatory region association facilitates the modulation of expression of sequences of interest that are operably linked to a given regulatory region by the associated regulatory protein. The regulatory protein associated with the regulatory region operably linked to the sequence of interest is itself operably linked to a regulatory region. The amount and specificity of expression of a regulatory protein can be modulated by selecting an appropriate regulatory region to direct expression of the regulatory protein. For example, a regulatory protein can be broadly expressed under the direction of a promoter such as a CaMV 35S promoter. Once expressed, the regulatory protein can modulate expression of a sequence of interest operably linked to another regulatory region, which is associated with the regulatory protein. In some cases, a regulatory protein can be expressed under the direction of a cell type- or tissue-preferential promoter, such as a cell type- or tissue-preferential promoter described below. In some embodiments, a regulatory region useful in the methods described herein has 80% or greater, e.g., 85%, 90%, 95%, 97%, 98%, 99%, or 100%, sequence identity to a regulatory region set forth in SEQ ID NOs:22-37.

The methods described herein can also be used to identify new regulatory region-regulatory protein association pairs. For example, an ortholog to a given regulatory protein is expected to associate with the associated regulatory region for that regulatory protein.

It should be noted that for a given regulatory protein listed in Table 4 (under Example 5 below), a regulatory region construct that includes one or more regulatory regions is set forth. A regulatory protein is expected to associate with either one or both such regulatory regions. Similarly, FIGS. 1-2 provide ortholog/homolog sequences and consensus sequences for corresponding regulatory proteins. It is contemplated that each such ortholog/homolog sequence and each polypeptide sequence that corresponds to the consensus sequence of the regulatory protein would also associate with the regulatory regions associated with the given regulatory protein as set forth in Table 4 (under Example 5 below).

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site, or about 2,000 nucleotides upstream of the transcription start site. A promoter typically comprises at least a core (basal) promoter. A promoter also may include at least one control element, such as an enhancer sequence, an upstream element or an upstream activation region (UAR). For example, a suitable enhancer is a cis-regulatory element (−212 to −154) from the upstream region of the octopine synthase (ocs) gene. Fromm et al., The Plant Cell, 1:977-984 (1989). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Some suitable promoters initiate transcription only, or predominantly, in certain cell types. For example, a promoter that is active predominantly in a reproductive tissue (e.g., fruit, ovule, pollen, pistils, female gametophyte, egg cell, central cell, nucellus, suspensor, synergid cell, flowers, embryonic tissue, embryo sac, embryo, zygote, endosperm, integument, or seed coat) can be used. Thus, as used herein a cell type- or tissue-preferential promoter is one that drives expression preferentially in the target tissue, but may also lead to some expression in other cell types or tissues as well. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110: 1069-1079 (1996).

Examples of various classes of promoters are described below. Some of the promoters indicated below are described in more detail in U.S. Patent Application Ser. Nos. 60/505,689; 60/518,075; 60/544,771; 60/558,869; 60/583,691; 60/619,181; 60/637,140; 10/950,321; 10/957,569; 11/058,689; 11/172,703; 11/208,308; and PCT/US05/23639. Nucleotide sequences of promoters are set forth in SEQ ID NOs:38-122. It will be appreciated that a promoter may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326 (SEQ ID NO:113), YP0144 (SEQ ID NO:92), YP0190 (SEQ ID NO:96), p13879 (SEQ ID NO:112), YP0050 (SEQ ID NO:72), p32449 (SEQ ID NO:114), 21876 (SEQ ID NO:38), YP0158 (SEQ ID NO:94), YP0214 (SEQ ID NO:98), YP0380 (SEQ ID NO:107), PT0848 (SEQ ID NO:63), and PT0633 (SEQ ID NO:44) promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128 (SEQ ID NO:89), YP0275 (SEQ ID NO:100), PT0625 (SEQ ID NO:43), PT0660 (SEQ ID NO:46), PT0683 (SEQ ID NO:51), and PT0758 (SEQ ID NO:59) promoters. Other root-preferential promoters include the PT0613 (SEQ ID NO:42), PT0672 (SEQ ID NO:48), PT0688 (SEQ ID NO:52), and PT0837 (SEQ ID NO:61) promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters reported by Conkling et al., Plant Physiol., 93:1203-1211 (1990), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell Biol., 13:5829-5842 (1993)), the beta-amylase promoter, and the barley hordein promoter. Other maturing endosperm promoters include the YP0092 (SEQ ID NO:75), PT0676 (SEQ ID NO:49), and PT0708 (SEQ ID NO:54) promoters.

Ovary Tissue Promoters

Promoters that are active in ovary tissues such as the ovule wall and mesocarp can also be useful, e.g., a polygalacturonidase promoter, the banana TRX promoter, and the melon actin promoter. Examples of promoters that are active primarily in ovules include YP0007 (SEQ ID NO:67), YP0111 (SEQ ID NO:83), YP0092 (SEQ ID NO:75), YP0103 (SEQ ID NO:80), YP0028 (SEQ ID NO:70), YP0121 (SEQ ID NO:88), YP0008 (SEQ ID NO:68), YP0039 (SEQ ID NO:71), YP0115 (SEQ ID NO:84), YP0119 (SEQ ID NO:86), YP0120 (SEQ ID NO:87), and YP0374 (SEQ ID NO:105).

Embryo Sac/Early Endosperm Promoters

To achieve expression in embryo sac/early endosperm, regulatory regions can be used that are active in polar nuclei and/or the central cell, or in precursors to polar nuclei, but not in egg cells or precursors to egg cells. Most suitable are promoters that drive expression only or predominantly in polar nuclei or precursors thereto and/or the central cell. A pattern of transcription that extends from polar nuclei into early endosperm development can also be found with embryo sac/early endosperm-preferential promoters, although transcription typically decreases significantly in later endosperm development during and after the cellularization phase. Expression in the zygote or developing embryo typically is not present with embryo sac/early endosperm promoters.

Promoters that may be suitable include those derived from the following genes: Arabidopsis viviparous-1 (see, GenBank No. U93215); Arabidopsis atmycl (see, Urao (1996) Plant Mol. Biol., 32:571-57; Conceicao (1994) Plant, 5:493-505); Arabidopsis FIE (GenBank No. AF 129516); Arabidopsis MEA; Arabidopsis FIS2 (GenBank No. AF096096); and FIE 1.1 (U.S. Pat. No. 6,906,244). Other promoters that may be suitable include those derived from the following genes: maize MAC1 (see, Sheridan (1996) Genetics, 142:1009-1020); maize Cat3 (see, GenBank No. L05934; Abler (1993) Plant Mol. Biol., 22:10131-1038). Other promoters include the following Arabidopsis promoters: YP0039 (SEQ ID NO:71), YP0101 (SEQ ID NO:78), YP0102 (SEQ ID NO:79), YP0110 (SEQ ID NO:82), YP0117 (SEQ ID NO:85), YP0119 (SEQ ID NO:86), YP0137 (SEQ ID NO:90), DME, YP0285 (SEQ ID NO:101), and YP0212 (SEQ ID NO:97). Other promoters that may be useful include the following rice promoters: p530c10, pOsFIE2-2, pOsMEA, pOsYp102, and pOsYp285.

Embryo Promoters

Regulatory regions that preferentially drive transcription in zygotic cells following fertilization can provide embryo-preferential expression. Most suitable are promoters that preferentially drive transcription in early stage embryos prior to the heart stage, but expression in late stage and maturing embryos is also suitable. Embryo-preferential promoters include the barley lipid transfer protein (Ltpl) promoter (Plant Cell Rep (2001) 20:647-654), YP0097 (SEQ ID NO:77), YP0107 (SEQ ID NO:81), YP0088 (SEQ ID NO:74), YP0143 (SEQ ID NO:91), YP0156 (SEQ ID NO:93), PT0650 (SEQ ID NO:45), PT0695 (SEQ ID NO:53), PT0723 (SEQ ID NO:56), PT0838 (SEQ ID NO:62), PT0879 (SEQ ID NO:65), and PT0740 (SEQ ID NO:57).

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535 (SEQ ID NO:40), PT0668 (SEQ ID NO:39), PT0886 (SEQ ID NO:66), YP0144 (SEQ ID NO:92), YP0380 (SEQ ID NO:107), and PT0585 (SEQ ID NO41).

Vascular Tissue Promoters

Examples of promoters that have high or preferential activity in vascular bundles include YP0087 (SEQ ID NO:116), YP0093 (SEQ ID NO:117), YP0108 (SEQ ID NO:118), YP0022 (SEQ ID NO:119), and YP0080 (SEQ ID NO:120). Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Poppy Capsule Promoters

Examples of promoters that have high or preferential activity in siliques/fruits, which are botanically equivalent to capsules in opium poppy, include PT0565 (SEQ ID NO:121) and YP0015 (SEQ ID NO:122).

Inducible Promoters

Inducible promoters confer transcription in response to external stimuli such as chemical agents or environmental stimuli. For example, inducible promoters can confer transcription in response to hormones such as gibberellic acid or ethylene, or in response to light or drought. Examples of drought-inducible promoters include YP0380 (SEQ ID NO:107), PT0848 (SEQ ID NO:63), YP0381 (SEQ ID NO:108), YP0337 (SEQ ID NO:103), PT0633 (SEQ ID NO:44), YP0374 (SEQ ID NO:105), PT0710 (SEQ ID NO:55), YP0356 (SEQ ID NO:104), YP0385 (SEQ ID NO:110), YP0396 (SEQ ID NO:111), YP0388, YP0384 (SEQ ID NO:109), PT0688 (SEQ ID NO:52), YP0286 (SEQ ID NO:102), YP0377 (SEQ ID NO:106), PD1367 (SEQ ID NO:115), PD0901, and PD0898. Nitrogen-inducible promoters include PT0863 (SEQ ID NO:64), PT0829 (SEQ ID NO:60), PT0665 (SEQ ID NO:47), and PT0886 (SEQ ID NO:66).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Other Promoters

Other classes of promoters include, but are not limited to, leaf-preferential, stem/shoot-preferential, callus-preferential, guard cell-preferential, such as PT0678 (SEQ ID NO:50), and senescence-preferential promoters. Promoters designated YP0086 (SEQ ID NO:73), YP0188 (SEQ ID NO:95), YP0263 (SEQ ID NO:99), PT0758 (SEQ ID NO:59), PT0743 (SEQ ID NO:58), PT0829 (SEQ ID NO:60), YP0119 (SEQ ID NO:86), and YP0096 (SEQ ID NO:76), as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a regulatory protein.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

Sequences of Interest and Plants and Plant Cells Containing the Same

Plant cells and plants described herein are useful because expression of a sequence of interest can be modulated to achieve a desired amount and/or specificity in expression by selecting an appropriate association of regulatory region and regulatory protein. A sequence of interest operably linked to a regulatory region can encode a polypeptide or can regulate the expression of a polypeptide. In some embodiments, a sequence of interest is transcribed into an anti-sense molecule. In some embodiments, more than one sequence of interest is present in a plant, e.g., two, three, four, five, six, seven, eight, nine, or ten sequences of interest. Each sequence of interest can be present on the same nucleic acid construct in such embodiments. Alternatively, each sequence of interest can be present on separate nucleic acid constructs. The regulatory region operably linked to each sequence of interest can be the same or can be different. In addition, one or more nucleotide sequences encoding a regulatory protein can be included on a nucleic acid construct that is the same as or separate from that containing an associated regulatory region(s) operably linked to a sequence(s) of interest. The regulatory region operably linked to each sequence encoding a regulatory protein can be the same or different.

A sequence of interest that encodes a polypeptide can encode a plant polypeptide, a non-plant polypeptide, e.g., a mammalian polypeptide, a modified polypeptide, a synthetic polypeptide, or a portion of a polypeptide. A sequence of interest can be endogenous, i.e., unmodified by recombinant DNA technology from the sequence and structural relationships that occur in nature and operably linked to the unmodified regulatory region. Alternatively, a sequence of interest can be an exogenous nucleic acid.

Alkaloid Biosynthesis Sequences

In certain cases, a sequence of interest can be an endogenous or exogenous sequence associated with alkaloid biosynthesis. For example, a transgenic plant cell containing a recombinant nucleic acid encoding a regulatory protein can be effective for modulating the amount and/or rate of biosynthesis of one or more alkaloid compounds. Such effects on alkaloid compounds typically occur via modulation of expression of one or more endogenous or exogenous sequences of interest operably linked to an associated regulatory region, e.g., endogenous sequences involved in alkaloid biosynthesis, such as native enzymes or regulatory proteins in alkaloid biosynthesis pathways, or exogenous sequences involved in alkaloid biosynthesis pathways introduced via a recombinant nucleic acid construct into a plant cell.

In some embodiments, the coding sequence can encode a polypeptide involved in alkaloid biosynthesis, e.g., an enzyme involved in biosynthesis of the alkaloid compounds described herein, or a regulatory protein involved in the biosynthesis pathways of the alkaloid compounds described herein. Other components that may be present in a sequence of interest include introns, enhancers, upstream activation regions, and inducible elements.

A suitable sequence of interest can encode an enzyme involved in tetrahydrobenzylisoquinoline alkaloid biosynthesis, e.g., selected from the group consisting of those encoding for tyrosine decarboxylase (YDC or TYD; EC 4.1.1.25), norcoclaurine synthase (EC 4.2.1.78), coclaurine N-methyltransferase (EC 2.1.1.140), (R,S)-norcoclaurine 6-O-methyl transferase (NOMT; EC 2.1.1.128), S-adenosyl-L-methionine:3′-hydroxy-N-methylcoclaurine 4′-O-methyltransferase 1 (HMCOMT1; EC 2.1.1.116); S-adenosyl-L-methionine:3′-hydroxy-N-methylcoclaurine 4′-O-methyltransferase 2 (HMCOMT2; EC 2.1.1.116); monophenol monooxygenase (EC 1.14.18.1), N-methylcoclaurine 3′-hydroxylase (NMCH EC 1.14.13.71), (R,S)-reticuline 7-O-methyltransferase (ROMT); berbamunine synthase (EC 1.14.21.3), columbamine O-methyltransferase (EC 2.1.1.118), berberine bridge enzyme (BBE; (EC 1.21.3.3), reticuline oxidase (EC 1.21.3.4), dehydro reticulinium ion reductase (EC 1.5.1.27), (RS)-1-benzyl-1,2,3,4-tetrahydroisoquinoline N-methyltransferase (EC 2.1.1.115), (S)-scoulerine oxidase (EC 1.14.21.2), (S)-cheilanthifoline oxidase (EC 1.14.21.1), (S)-tetrahydroprotoberberine N-methyltransferase (EC 2.1.1.122), (S)-canadine synthase (EC 1.14.21.5), tetrahydroberberine oxidase (EC 1.3.3.8), columbamine oxidase (EC 1.21.3.2), and other enzymes, such as protopine-6-monooxygenase, related to the biosynthesis of tetrahydrobenzylisoquinoline alkaloids.

In other cases, a sequence of interest can be an enzyme involved in benzophenanthridine alkaloid biosynthesis, e.g., selected from the group consisting of those encoding for dihydrobenzophenanthridine oxidase (EC 1.5.3.12), dihydrosanguinarine 10-hydroxylase (EC 1.14.13.56), 10-hydroxydihydrosanguinarine 10-O-methyltransferase (EC 2.1.1.119), dihydrochelirubine 12-hydroxylase ( EC 1.14.13.57), 12-hydroxydihydrochelirubine 12-O-methyltransferase (EC 2.1.1.120), and other enzymes, including dihydrobenzophenanthridine oxidase and dihydrosanguinarine 10-monooxygenase, related to the biosynthesis of benzophenanthridine alkaloids.

In yet other cases, a sequence is involved in morphinan alkaloid biosynthesis, e.g., selected from the group consisting of salutaridinol 7-O-acetyltransferase (SAT; EC 2.3.1.150), salutaridine synthase (EC 1.14.21.4), salutaridine reductase (EC 1.1.1.248), morphine 6-dehydrogenase (EC 1.1.1.218); and codeinone reductase (CR; EC 1.1.1.247); and other sequences related to the biosynthesis of morphinan/opiate alkaloids.

In other embodiments, a suitable sequence encodes an enzyme involved in purine alkaloid (e.g., xanthines, such as caffeine) biosynthesis such as xanthosine methyltransferase, 7-N-methylxanthine methyltransferase (theobromine synthase), or 3,7-dimethylxanthine methyltransferase (caffeine synthase).

In some embodiments, a suitable sequence encodes an enzyme involved in biosynthesis of indole alkaloids compounds such as tryptophane decarboxylase, strictosidine synthase, strictosidine glycosidase, dehydrogeissosshizine oxidoreductase, polyneuridine aldehyde esterase, sarpagine bridge enzyme, vinorine reductase, vinorine synthase, vinorine hydroxylase, 17-O-acetylajmalan acetylesterase, or norajamaline N-methyl transferase. In other embodiments, a suitable sequence of interest encodes an enzyme involved in biosynthesis of vinblastine, vincristine and compounds derived from them, such as tabersonine 16-hydroxylase, 16-hydroxytabersonine 16-O-methyl transferase, desacetoxyvindoline 4-hydroxylase, or desacetylvindoline O-acetyltransferasesynthase.

In still other embodiments, a suitable sequence encodes an enzyme involved in biosynthesis of pyridine, tropane, and/or pyrrolizidine alkaloids such as arginine decarboxylase, spermidine synthase, ornithine decarboxylase, putrescine N-methyl transferase, tropinone reductase, hyoscyamine 6-beta-hydroxylase, diamine oxidase, and tropinone dehydrogenase.

Other Sequences of Interest

Other sequences of interest can encode a therapeutic polypeptide for use with mammals such as humans, e.g., as set forth in Table 1, below. In certain cases, a sequence of interest can encode an antibody or antibody fragment. An antibody or antibody fragment includes a humanized or chimeric antibody, a single chain Fv antibody fragment, an Fab fragment, and an F(ab)₂ fragment. A chimeric antibody is a molecule in which different portions are derived from different animal species, such as those having a variable region derived from a mouse monoclonal antibody and a human immunoglobulin constant region. Antibody fragments that have a specific binding affinity can be generated by known techniques. Such antibody fragments include, but are not limited to, F(ab′)₂ fragments that can be produced by pepsin digestion of an antibody molecule, and Fab fragments that can be generated by deducing the disulfide bridges of F(ab′)₂ fragments. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778. U.S. Pat. No. 6,303,341 discloses immunoglobulin receptors. U.S. Pat. No. 6,417,429 discloses immunoglobulin heavy- and light-chain polypeptides.

TABLE 1
Human Therapeutic Proteins

| | | |
|---|---|---|
| Bromelain | Humatrope ® | Proleukin ® |
| Chymopapain | Humulin ® (insulin) | Protropin ® |
| Papain ® | Infergen ® | Recombivax-HB ® |
| Activase ® | Interferon-gamma-1a | Recormon ® |
| Albutein ® | Interleukin-2 | Remicade ® (s-TNF-r) |
| Angiotensin II | Intron ® | ReoPro ® |
| Asparaginase | Leukine ® (GM-CSF) | Retavase ® (TPA) |
| Avonex ® | Nartogastrim ® | Roferon-A ® |
| Betaseron ® | Neumega ® | Pegaspargas |
| BioTropin ® | Neupogen ® | Prandin ® |
| Cerezyme ® | Norditropin ® | Procrit ® |
| Enbrel ® (s-TNF-r) | Novolin ® (insulin) | Filgastrim ® |
| Engerix-B ® | Nutropin ® | Genotropin ® |
| Epogen ® | Oncaspar ® | Geref ® |
| Sargramostrim | Tripedia ® | Trichosanthin |
| TriHIBit ® | Venoglobin-S ® (HIG) | |

A sequence of interest can encode a polypeptide or result in a transcription product anti-sense molecule that confers insect resistance, bacterial disease resistance, fungal disease resistance, viral disease resistance, nematode disease resistance, herbicide resistance, enhanced grain composition or quality, enhanced nutrient composition, nutrient transporter functions, enhanced nutrient utilization, enhanced environmental stress tolerance, reduced mycotoxin contamination, female sterility, a selectable marker phenotype, a screenable marker phenotype, a negative selectable marker phenotype, or altered plant agronomic characteristics. Specific examples include, without limitation, a chitinase coding sequence and a glucan endo-1,3-β-glucosidase coding sequence. In some embodiments, a sequence of interest encodes a bacterial ESPS synthase that confers resistance to glyphosate herbicide or a phosphinothricin acetyl transferase coding sequence that confers resistance to phosphinothricin herbicide.

A sequence of interest can encode a polypeptide involved in the production of industrial or pharmaceutical chemicals, modified and specialty oils, enzymes, or renewable non-foods such as fuels and plastics, vaccines and antibodies. U.S. Pat. No. 5,824,779 discloses phytase-protein-pigmenting concentrate derived from green plant juice. U.S. Pat. No. 5,900,525 discloses animal feed compositions containing phytase derived from transgenic alfalfa. U.S. Pat. No. 6,136,320 discloses vaccines produced in transgenic plants. U.S. Pat. No. 6,255,562 discloses insulin. U.S. Pat. No. 5,958,745 discloses the formation of copolymers of 3-hydroxy butyrate and 3-hydroxy valerate. U.S. Pat. No. 5,824,798 discloses starch synthases. U.S. Pat. No. 6,087,558 discloses the production of proteases in plants. U.S. Pat. No. 6,271,016 discloses an anthranilate synthase gene for tryptophan overproduction in plants.

Methods of Inhibiting Expression of a Sequence of Interest

The polynucleotides and recombinant vectors described herein can be used to express or inhibit expression of a gene, such as an endogenous gene involved in alkaloid biosynthesis, e.g., to alter alkaloid biosynthetic pathways in a plant species of interest. The term “expression” refers to the process of converting genetic information of a polynucleotide into RNA through transcription, which is catalyzed by an enzyme, RNA polymerase, and into protein, through translation of mRNA on ribosomes. “Up-regulation” or “activation” refers to regulation that increases the production of expression products (mRNA, polypeptide, or both) relative to basal or native states, while “down-regulation” or “repression” refers to regulation that decreases production of expression products (mRNA, polypeptide, or both) relative to basal or native states.

“Modulated level of gene expression” as used herein refers to a comparison of the level of expression of a transcript of a gene or the amount of its corresponding polypeptide in the presence and absence of a regulatory protein described herein, and refers to a measurable or observable change in the level of expression of a transcript of a gene or the amount of its corresponding polypeptide relative to a control plant or plant cell under the same conditions (e.g., as measured through a suitable assay such as quantitative RT-PCR, a “northern blot,” a “western blot” or through an observable change in phenotype, chemical profile, or metabolic profile). A modulated level of gene expression can include up-regulated or down-regulated expression of a transcript of a gene or polypeptide relative to a control plant or plant cell under the same conditions. Modulated expression levels can occur under different environmental or developmental conditions or in different locations than those exhibited by a plant or plant cell in its native state.

A number of nucleic acid based methods, including antisense RNA, co-suppression, ribozyme directed RNA cleavage, and RNA interference (RNAi) can be used to inhibit protein expression in plants. Antisense technology is one well-known method. In this method, a nucleic acid segment from a gene to be repressed is cloned and operably linked to a promoter so that the antisense strand of RNA is transcribed. The recombinant vector is then transformed into plants, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Constructs containing operably linked nucleic acid molecules in the sense orientation can also be used to inhibit the expression of a gene. The transcription product can be similar or identical to the sense coding sequence of a polypeptide of interest. The transcription product can also be unpolyadenylated, lack a 5′ cap structure, or contain an unsplicable intron. Methods of co-suppression using a full-length cDNA as well as a partial cDNA sequence are known in the art. See, e.g., U.S. Pat. No. 5,231,020.

In another method, a nucleic acid can be transcribed into a ribozyme, or catalytic RNA, that affects expression of an mRNA. (See, U.S. Pat. No. 6,423,885). Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. No. 4,987,071 and 6,423,885.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art. See, e.g., U.S. Pat. Nos. 5,034,323; 6,326,527; 6,452,067; 6,573,099; 6,753,139; and 6,777,588. See also WO 97/01952; WO 98/53083; WO 99/32619; WO 98/36083; and U.S. Patent Publications 20030175965, 20030175783, 20040214330, and 20030180945.

In some nucleic-acid based methods for inhibition of gene expression in plants, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller, 1997, Antisense Nucleic Acid Drug Dev., 7:187-195; Hyrup et al., Bioorgan. Med. Chem., 4:5-23 (1996). In addition, the deoxyphosphate backbone can be replaced with, for example, a phosphorothioate or phosphorodithioate backbone, a phosphoroamidite, or an alkyl phosphotriester backbone.

Transgenic Plant Cells and Plants

Provided herein are transgenic plant cells and plants comprising at least one recombinant nucleic acid construct or exogenous nucleic acid. A recombinant nucleic acid construct or exogenous nucleic acid can include a regulatory region as described herein, a nucleic acid encoding a regulatory protein as described herein, or both. In certain cases, a transgenic plant cell or plant comprises at least two recombinant nucleic acid constructs or exogenous nucleic acids, one including a regulatory region, and one including a nucleic acid encoding the associated regulatory protein.

A plant or plant cell used in methods of the invention contains a recombinant nucleic acid construct as described herein. A plant or plant cell can be transformed by having a construct integrated into its genome, i.e., can be stably transformed. Stably transformed cells typically retain the introduced nucleic acid with each cell division. A plant or plant cell can also be transiently transformed such that the construct is not integrated into its genome. Transiently transformed cells typically lose all or some portion of the introduced nucleic acid construct with each cell division such that the introduced nucleic acid cannot be detected in daughter cells after a sufficient number of cell divisions. Both transiently transformed and stably transformed transgenic plants and plant cells can be useful in the methods described herein.

Typically, transgenic plant cells used in methods described herein constitute part or all of a whole plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species or for further selection of other desirable traits. Alternatively, transgenic plants can be propagated vegetatively for those species amenable to such techniques. Progeny includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. Seeds produced by a transgenic plant can be grown and then selfed (or outcrossed and selfed) to obtain seeds homozygous for the nucleic acid construct.

Transgenic plant cells growing in suspension culture, or tissue or organ culture, can be useful for extraction of alkaloid compounds. For the purposes of this invention, solid and/or liquid tissue culture techniques can be used. When using solid medium, transgenic plant cells can be placed directly onto the medium or can be placed onto a filter film that is then placed in contact with the medium. When using liquid medium, transgenic plant cells can be placed onto a floatation device, e.g., a porous membrane that contacts the liquid medium. Solid medium typically is made from liquid medium by adding agar. For example, a solid medium can be Murashige and Skoog (MS) medium containing agar and a suitable concentration of an auxin, e.g., 2,4-dichlorophenoxyacetic acid (2,4-D), and a suitable concentration of a cytokinin, e.g., kinetin.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous regulatory protein whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, e.g., U.S. Pat. Nos. 5,538,880, 5,204,253, 6,329,571 and 6,013,863. If a cell or tissue culture is used as the recipient tissue for transformation, plants can be regenerated from transformed cultures if desired, by techniques known to those skilled in the art. See, e.g., Allen et al., “RNAi-mediated replacement of morphine with the nonnarcotic alkaloid reticuline in opium poppy,” Nature Biotechnology 22(12):1559-1566 (2004); Chitty et al., “Genetic transformation in commercial Tasmanian cultures of opium poppy, Papaver somniferum, and movement of transgenic pollen in the field,” Funct. Plant Biol. 30:1045-1058 (2003); and Park et al., J. Exp. Botany 51(347):1005-1016 (2000).

Plant Species

The polynucleotides and vectors described herein can be used to transform a number of monocotyledonous and dicotyledonous plants and plant cell systems. A suitable group of plant species includes dicots, such as poppy, safflower, alfalfa, soybean, cotton, coffee, rapeseed (high erucic acid and canola), or sunflower. Also suitable are monocots such as corn, wheat, rye, barley, oat, rice, millet, amaranth or sorghum. Also suitable are vegetable crops or root crops such as lettuce, carrot, onion, broccoli, peas, sweet corn, popcorn, tomato, potato, beans (including kidney beans, lima beans, dry beans, green beans) and the like. Also suitable are fruit crops such as grape, strawberry, pineapple, melon (e.g., watermelon, cantaloupe), peach, pear, apple, cherry, orange, lemon, grapefruit, plum, mango, banana, and palm.

Thus, the methods and compositions described herein can be utilized with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myriales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. Methods described herein can also be utilized with monocotyledonous plants belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales, or with plants belonging to Gymnospermae, e.g., Pinales, Ginkgoales, Cycadales and Gnetales.

The invention has use over a broad range of plant species, including species from the genera Allium, Alseodaphne, Anacardium, Arachis, Asparagus, Atropa, Avena, Beilschmiedia, Brassica, Citrus, Citrullus, Capsicum, Catharanthus, Carthamus, Cocculus, Cocos, Coffea, Croton, Cucumis, Cucurbita, Daucus, Duguetia, Elaeis, Eschscholzia, Ficus, Fragaria, Glaucium, Glycine, Gossypium, Helianthus, Heterocallis, Hevea, Hordeum, Hyoscyamus, Lactuca, Landolphia, Linum, Litsea, Lolium, Lupinus, Lycopersicon, Malus, Manihot, Majorana, Medicago, Musa, Nicotiana, Olea, Oryza, Panicum, Pannesetum, Papaver, Parthenium, Persea, Phaseolus, Pinus, Pistachia, Pisum, Pyrus, Prunus, Raphanus, Rhizocarya, Ricinus, Secale, Senecio, Sinomenium, Sinapis, Solanum, Sorghum, Stephania, Theobroma, Trigonella, Triticum, Vicia, Vinca, Vitis, Vigna, and Zea.

Particularly suitable plants with which to practice the invention include plants that are capable of producing one or more alkaloids. A “plant that is capable of producing one or more alkaloids” refers to a plant that is capable of producing one or more alkaloids even when it is not transgenic for a regulatory protein described herein. For example, a plant from the Solanaceae or Papaveraceae family is capable of producing one or more alkaloids when it is not transgenic for a regulatory protein described herein. In certain cases, a plant or plant cell may be transgenic for sequences other than the regulatory protein sequences described herein, e.g., growth factors or stress modulators, and can still be characterized as “capable of producing one or more alkaloids,” e.g., a Solanaceae family member transgenic for a growth factor but not transgenic for a regulatory protein described herein.

Useful plant families that are capable of producing one or more alkaloids include the Papaveraceae, Berberidaceae, Lauraceae, Menispermaceae, Euphorbiaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, and Ruiaceae families. The Papaveraceae family, for example, contains about 250 species found mainly in the northern temperate regions of the world and includes plants such as California poppy and Opium poppy. Useful genera within the Papaveraceae family include the Papaver (e.g., Papaver bracteatum, Papaver orientale, Papaver setigerum, and Papaver somniferum), Sanguinaria, Dendromecon, Glaucium, Meconopsis, Chelidonium, Eschscholzioideae (e.g., Eschscholzia, Eschscholzia california), and Argemone (e.g., Argemone hispida, Argemone mexicana, and Argemone munita) genera. Other alkaloid producing species with which to practice this invention include Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, and Beilschmiedia oreophila, or other species listed in Table 2, below.

Alkaloid Compounds

Compositions and methods described herein are useful for producing one or more alkaloid compounds. Alkaloid compounds are nitrogenous organic molecules that are typically derived from plants. Alkaloid biosynthetic pathways often include amino acids as reactants. Alkaloid compounds can be mono-, bi-, or polycyclic compounds. Bi- or poly-cyclic compounds can include bridged structures or fused rings. In certain cases, an alkaloid compound can be a plant secondary metabolite.

The regulatory proteins described previously can modulate expression of sequences involved in the biosynthesis of alkaloid compounds. Thus, a transgenic plant or cell comprising a recombinant nucleic acid expressing such a regulatory protein can be effective for modulating the amount and/or rate of biosynthesis of one or more of such alkaloids in a plant containing the associated regulatory region, either as a genomic sequence or introduced in a recombinant nucleic acid construct.

An amount of one or more of any individual alkaloid compound can be modulated, e.g., increased or decreased, relative to a control plant or cell not transgenic for the particular regulatory protein using the methods described herein. In certain cases, therefore, more than one alkaloid compound (e.g., two, three, four, five, six, seven, eight, nine, ten or even more alkaloid compounds) can have its amount modulated relative to a control plant or cell that is not transgenic for a regulatory protein described herein.

Alkaloid compounds can be grouped into classes based on chemical and structural features. Alkaloid classes described herein include, without limitation, tetrahydrobenzylisoquinoline alkaloids, morphinan alkaloids, benzophenanthridine alkaloids, monoterpenoid indole alkaloids, bisbenzylisoquinoline alkaloids, pyridine alkaloids, purine alkaloids, tropane alkaloids, quinoline alkaloids, terpenoid alkaloids, betaine alkaloids, steroid alkaloids, acridone alkaloids, and phenethylamine alkaloids. Other classifications may be known to those having ordinary skill in the art. Alkaloid compounds whose amounts are modulated relative to a control plant can be from the same alkaloid class or from different alkaloid classes.

In certain embodiments, a morphinan alkaloid compound that is modulated is salutaridine, salutaridinol, salutaridinol acetate, thebaine, isothebaine, papaverine, narcotine, narceine, hydrastine, oripavine, morphinone, morphine, codeine, codeinone, and neopinone. Other morphinan analog alkaloid compounds of interest include sinomenine, flavinine, oreobeiline, and zipperine.

In other embodiments, a tetrahydrobenzylisoquinoline alkaloid compound that is modulated is noscapine, 2′-norberbamunine, S-coclaurine, S-norcoclaurine, R-N-methyl-coclaurine, S—N-methylcoclaurine, S-3′-hydroxy-N-methylcoclaurine, aromarine, S-3-hydroxycoclaurine, S-norreticuline, R-norreticuline, S-reticuline, R-reticuline, S-scoulerine, S-cheilanthifoline, S-stylopine, S-cis-N-methyl-stylopine, protopine, 6-hydroxy-protopine, 1,2-dehydro-reticuline, S-tetrahydrocolumbamine, columbamine, palmatine, tetrahydropalmatine, S-canadine, berberine, S-norlaudenosoline, 6-O-methylnorlaudanosoline, and nororientaline.

In some embodiments, a benzophenanthridine alkaloid compound can be modulated, which can be dihydrosanguinarine, sanguinarine, dihydroxy-dihydro-sanguinarine, 12-hydroxy-dihydrochelirubine, 10-hydroxy-dihydro-sanguinarine, dihydro-macarpine, dihydro-chelirubine, dihydro-sanguinarine, chelirubine, 12-hydroxy-chelirubine, or macarpine.

In yet other embodiments, monoterpenoid indole alkaloid compounds that are modulated include vinblastine, vincristine, yohimbine, ajmalicine, ajmaline, and vincamine. In other cases, a pyridine alkaloid is modulated. A pyridine alkaloid can be piperine, coniine, trigonelline, arecaidine, guvacine, pilocarpine, cytosine, nicotine, and sparteine. A tropane alkaloid that can be modulated includes atropine, cocaine, tropacocaine, hygrine, ecgonine, (−) hyoscyamine, (−) scopolamine, and pelletierine. A quinoline alkaloid that is modulated can be quinine, strychnine, brucine, veratrine, or cevadine. Acronycine is an example of an acridone alkaloid.

In some cases, a phenylethylamine alkaloid can be modulated, which can be MDMA, methamphetamine, mescaline, and ephedrine. In other cases, a purine alkaloid is modulated, such as the xanthines caffeine, theobromine, theacrine, and theophylline.

Bisbenzylisoquinoline alkaloids that can be modulated in amount include (+)tubocurarine, dehatrine, (+)thalicarpine, aromoline, guatteguamerine, berbamunine, and isotetradine. Yet another alkaloid compound that can be modulated in amount is 3,4-dihydroxyphenylacetaldehyde.

Certain useful alkaloid compounds, with associated plant species that are capable of producing them, are listed in Table 2, below.

TABLE 2
Alkaloid Compound Table

| Alkaloid Name | Plant Source(s) |
|---|---|
| Apomorphine | Papaver somniferum |
| Hemsleyadine | Aconitum hemsleyanum, Hemsleya amabilis |
| Anabasine | Anabasis sphylla |
| Aconitine | Aconitum spp. |
| Anisodamine | Anisodus tanguticus |
| Anisodine | Datura sanguinera |
| Arecoline | Areca catechu |
| Atropine | Atropa belladonna, Datura stomonium |
| Homatropine | Atropa belladonna |
| Berberine | Berberis spp. and Mahonia spp. |
| Caffeine | Camellia sinensis, Theobroma cacao, Coffea |
| | arabica, Cola spp. |
| Camptothecin | Camptotheca acuminata |
| Orothecin | Camptotheca acuminata |
| 9-amino | Camptotheca acuminata |
| camptothecin | |
| Topotecan | Camptotheca acuminata |
| Irinotecan | Camptotheca acuminata |
| Castanospermine | Castanosperma australe, Alexa spp. |
| Vinblastine | Catharanthus roseus |
| Vincristine | Catharanthus roseus |
| Vinorelbine | Catharanthus roseus |
| Emetine | Alangium lamarkii, Cephaelis ipecacuanha, |
| | Psychotria spp. |
| Homoharringtonine | Cephalotaxus spp. |
| Harringtonine | Cephalotaxus spp. |
| Tubocurarine | Chondodendron tomentosum |
| Quinine | Cinchona officinalis, Cinchona spp., Remijia |
| | pedunculata |
| Quinidine | Cinchona spp., Remijia pedunculata |
| Cissampareine | Cissampelos pareira |
| Cabergoline | Claviceps pupurea |
| Colchicine | Colchicum autumnale |
| Demecolcine | Colchicum spp., Merendera spp. |
| Palmatine | Coptis japonica, Berberis spp., Mahonia spp. |
| Tetrahydropalmatine | Coptis japonica, Berberis spp., Mahonia spp. |
| Monocrotaline | Crotalaria spp. |
| Sparteine | Cytisus scoparius, Sophora pschycarpa, |
| | Ammodendron spp. |
| Changrolin | Dichroa febrifuga |
| Ephedrine | Ephedra sinica, Ephedra spp. |
| Cocaine | Erythroxylum coca |
| Rotundine | Eschsholtzia californica, Stephania sinica, |
| | Eschsholtzia spp., Argemone spp. |
| Galanthamine | Galanthus wornorii |
| Gelsemin | Gelsemium sempervivens |
| Glaucine | Glaucium flavum, Berberis spp. and Mahonia spp. |
| Indicine | Heliotropium indicum & Messerschmidia |
| | argentea |
| Hydrastine | Hydrastis canadensis |
| Hyoscyamine | Hyoscyamus, Atropa, Datura, Scopolia spp. |
| a-Lobeline | Lobelia spp. |
| Huperzine A | Lycopodium serratum (=Huperzia serrata), |
| | Lycopodium spp. |
| Ecteinascidin 743 | Marine tunicate - Ecteinascidia turbinata |
| Nicotine | Nicotiana tabacum |
| Ellipticine | Ochrosia spp., Aspidospera subincanum, |
| | Bleekeria vitiensis |
| 9- | Ochrosia spp., Excavatia coccinea, Bleekeria |
| Methoxyellipticine | vitiensis |
| Codeine | Papaver somniferum |
| Hydrocodone | Papaver somniferum |
| Hydromorphone | Papaver somniferum |
| Morphine | Papaver somniferum |
| Narceine | Papaver somniferum |
| Oxycodone | Papaver somniferum |
| Oxymorphone | Papaver somniferum |
| Papaverine | Papaver somniferum, Rauwolfia serpentina |
| Thebaine | Papaver bracteatum, Papaver spp. |
| Yohimbine | Pausinystalia yohimbe, Rauwolfia, Vinca, & |
| | Catharanthus spp. |
| Physostigmine | Physostigma venenosum |
| Pilocarpine | Pilocarpus microphyllus, Philocarpus spp. |
| Oxandrin | Pseudoxandra lucida |
| Sarpagine | Rauwolfia & Vinca spp. |
| Deserpidine | Rauwolfia canescens, Rauwolfia spp. |
| Rescinnamine | Rauwolfia spp. |
| Reserpine | Rauwolfia serpentina, Rauwolfia spp. |
| Ajmaline | Rauwolfia serpentina, Rauwolfia spp., Melodinus |
| | balansae, Tonduzia longifolia |
| Ajmalicine | Rauwolfia spp., Vinca rosea |
| Sanguinarine | Sanguinaria canadensis, Eschscholtzia californica |
| Matrine | Sophora spp. |
| Tetrandrine | Stephania tetrandra |
| Strychnine | Strychnos nux-vomica, Strychnos spp. |
| Brucine | Strychnos spp. |
| Protoveratrines A, B | Veratrum spp. |
| Cyclopamine | Vertatrum spp. |
| Veratramine | Veratrum spp. |
| Vasicine | Vinca minor, Galega officinalis |
| Vindesine | Vinca rosea |
| Vincamine | Vinca spp. |
| Buprenorphine | Papaver somniferum |
| Cimetropium | Atropa, Datura, Scopolia, Hyoscyamus spp. |
| Bromide | |
| Levallorphan | Papaver somniferum |
| Serpentine | Rauwolfvia spp. and Catharanthus spp. |
| Noscapine | Papaver somniferum |
| Scopolamine | Atropa, Datura, Scopolia, Hyoscyamus spp. |
| Salutaridine | Croton salutaris, Croton balsamifera, Papaver |
| | spp. and Glaucium spp. |
| Sinomenine | Sinomenium acutum and Stephania cepharantha |
| Flavinine | Litsea sebiferea, Alseodaphne perakensis, |
| | Cocculus laurifolius, Duguetia obovata and |
| | Rhizocarya racemifera |
| Oreobeiline | Beilschmiedia oreophila |
| Zippeline | Stephania zippeliana |

The amount of one or more alkaloid compounds can be increased or decreased in transgenic cells or tissues expressing a regulatory protein as described herein. An increase can be from about 1.5-fold to about 300-fold, or about 2-fold to about 22-fold, or about 50-fold to about 200-fold, or about 75-fold to about 130-fold, or about 5-fold to about 50-fold, or about 5-fold to about 10-fold, or about 10-fold to about 20-fold, or about 150-fold to about 200-fold, or about 20-fold to about 75-fold, or about 10-fold to about 100-fold, or about 40-fold to about 150-fold, about 100-fold to about 200-fold, about 150-fold to about 300-fold, or about 30-fold to about 50-fold higher than the amount in corresponding control cells or tissues that lack the recombinant nucleic acid encoding the regulatory protein.

In other embodiments, the alkaloid compound that is increased in transgenic cells or tissues expressing a regulatory protein as described herein is either not produced or is not detectable in corresponding control cells or tissues that lack the recombinant nucleic acid encoding the regulatory protein. Thus, in such embodiments, the increase in such an alkaloid compound is infinitely high as compared to corresponding control cells or tissues that lack the recombinant nucleic acid encoding the regulatory protein. For example, in certain cases, a regulatory protein described herein may activate a biosynthetic pathway in a plant that is not normally activated or operational in a control plant, and one or more new alkaloids that were not previously produced in that plant species can be produced.

The increase in amount of one or more alkaloids can be restricted in some embodiments to particular tissues and/or organs, relative to other tissues and/or organs. For example, a transgenic plant can have an increased amount of an alkaloid in leaf tissue relative to root or floral tissue.

In other embodiments, the amounts of one or more alkaloids are decreased in transgenic cells or tissues expressing a regulatory protein as described herein. A decrease ratio can be expressed as the ratio of the alkaloid in such a transgenic cell or tissue on a weight basis (e.g., fresh or freeze dried weight basis) as compared to the alkaloid in a corresponding control cell or tissue that lacks the recombinant nucleic acid encoding the regulatory protein. The decrease ratio can be from about 0.05 to about 0.90. In certain cases, the ratio can be from about 0.2 to about 0.6, or from about 0.4 to about 0.6, or from about 0.3 to about 0.5, or from about 0.2 to about 0.4.

In certain embodiments, the alkaloid compound that is decreased in transgenic cells or tissues expressing a regulatory protein as described herein is decreased to an undetectable level as compared to the level in corresponding control cells or tissues that lack the recombinant nucleic acid encoding the regulatory protein. Thus, in such embodiments, the decrease ratio in such an alkaloid compound is zero.

The decrease in amount of one or more alkaloids can be restricted in some embodiments to particular tissues and/or organs, relative to other tissues and/or organs. For example, a transgenic plant can have a decreased amount of an alkaloid in leaf tissue relative to root or floral tissue.

In some embodiments, the amounts of two or more alkaloids are increased and/or decreased, e.g., the amounts of two, three, four, five, six, seven, eight, nine, ten (or more) alkaloid compounds are independently increased and/or decreased. The amount of an alkaloid compound can be determined by known techniques, e.g., by extraction of alkaloid compounds followed by gas chromatography-mass spectrometry (GC-MS) or liquid chromatography-mass spectrometry (LC-MS). If desired, the structure of the alkaloid compound can be confirmed by GC-MS, LC-MS, nuclear magnetic resonance and/or other known techniques.

Methods of Screening for Associations and Modulating Expression of Sequences of Interest

Provided herein are methods of screening for novel regulatory region-regulatory protein association pairs. The described methods can thus determine whether or not a given regulatory protein can activate a given regulatory region (e.g., to modulate expression of a sequence of interest operably linked to the given regulatory region).

A method of determining whether or not a regulatory region is activated by a regulatory protein can include determining whether or not reporter activity is detected in a plant cell transformed with a recombinant nucleic acid construct comprising a test regulatory region operably linked to a nucleic acid encoding a polypeptide having the reporter activity and with a recombinant nucleic acid construct comprising a nucleic acid encoding a regulatory protein described herein. Detection of the reporter activity indicates that the test regulatory region is activated by the regulatory protein. In certain cases, the regulatory region is a regulatory region as described herein, e.g., comprising a nucleic acid sequence having 80% or greater sequence identity to a regulatory region as set forth in SEQ ID NOs:22-37.

For example, a plant can be made that is stably transformed with a sequence encoding a reporter operably linked to the regulatory region under investigation. The plant is inoculated with Agrobacterium containing a sequence encoding a regulatory protein on a Ti plasmid vector. A few days after inoculation, the plant tissue is examined for expression of the reporter, or for detection of reporter activity associated with the reporter. If reporter expression or activity is observed, it can be concluded that the regulatory protein increases expression of the reporter coding sequence. A positive result indicates that expression of the regulatory protein being tested in a plant would be effective for increasing the in planta amount and/or rate of biosynthesis of one or more sequences of interest operably linked to the associated regulatory region.

Similarly, a method of determining whether or not a regulatory region is activated by a regulatory protein can include determining whether or not reporter activity is detected in a plant cell transformed with a recombinant nucleic acid construct comprising a regulatory region as described herein operably linked to a reporter nucleic acid, and with a recombinant nucleic acid construct comprising a nucleic acid encoding a test regulatory protein. Detection of reporter activity indicates that the regulatory region is activated by the test regulatory protein. In certain cases, the regulatory protein is a regulatory protein as described herein, e.g., comprising a polypeptide sequence having 80% or greater sequence identity to a polypeptide sequence set forth in SEQ ID NOs:2-13, SEQ ID NOs:15-19, SEQ ID NO:21, or the consensus sequences set forth in FIG. 1-2.

A transformation can be a transient transformation or a stable transformation, as discussed previously. The regulatory region and the nucleic acid encoding a test regulatory protein can be on the same or different nucleic acid constructs.

A reporter activity, such as an enzymatic or optical activity, can permit the detection of the presence of the reporter polypeptide in situ or in vivo, either directly or indirectly. For example, a reporter polypeptide can itself be bioluminescent upon exposure to light. As an alternative, a reporter polypeptide can catalyze a chemical reaction in vivo that yields a detectable product that is localized inside or that is associated with a cell that expresses the chimeric polypeptide. Exemplary bioluminescent reporter polypeptides that emit light in the presence of additional polypeptides, substrates or cofactors include firefly luciferase and bacterial luciferase. Bioluminescent reporter polypeptides that fluoresce in the absence of additional proteins, substrates or cofactors when exposed to light having a wavelength in the range of 300 nm to 600 nm include, for example: amFP486, Mut15-amFP486, Mut32-amFP486, CNFP-MODCd1 and CNFP-MODCd2; asFP600, mut1-RNFP, NE-RNFP, d1RNFP and d2RNFP; cFP484, Δ19-cFP484 and Δ38-cFP484; dgFP512; dmFP592; drFP583, E5 drFP583, E8 drFP583, E5UP drFP583, E5down drFP583, E57 drFP583, AG4 drFP583 and AG4H drFP583; drFP583/dmFP592, drFP583/dmFP592-2G and drFP583/dmFP592-Q3; dsFP483; zFP506, N65M-zFP506, d1zFP506 and d2zFP506; zFP538, M128V-zFP538, YNFPM128V-MODCd1 and YNFPM128V-MODCd2; GFP; EGFP, ECFP, EYFP, EBFP, BFP2; d4EGFP, d2EGFP, and d1EGFP; and DsRed and DsRed1. See WO 00/34318; WO 00/34320; WO 00/34319; WO 00/34321; WO 00/34322; WO 00/34323; WO 00/34324; WO 00/34325; WO 00/34326; GenBank Accession No. AAB57606; Clontech User Manual, April 1999, PT2040-1, version PR94845; Li et al., J. Biol. Chem. 1998, 273:34970-5; U.S. Pat. No. 5,777,079; and Clontech User Manual, October 1999, PT34040-1, version PR9X217. Reporter polypeptides that catalyze a chemical reaction that yields a detectable product include, for example, β-galactosidase or β-glucuronidase. Other reporter enzymatic activities for use in the invention include neomycin phosphotransferase activity and phosphinotricin acetyl transferase activity.

In some cases, it is known that a particular regulatory protein can activate expression from a particular alkaloid regulatory region(s), e.g., a regulatory region involved in alkaloid biosynthesis. In these cases, similar methods can also be useful to screen other regulatory regions, such as other regulatory regions involved in alkaloid biosynthesis, to determine whether they are activated by the same regulatory protein. Thus, the method can comprise transforming a plant cell with a nucleic acid comprising a test regulatory region operably linked to a nucleic acid encoding a polypeptide having reporter activity. The plant cell can include a recombinant nucleic acid encoding a regulatory protein operably linked to a regulatory region that drives transcription of the regulatory protein in the cell. If reporter activity is detected, it can be concluded that the regulatory protein activates expression from the test regulatory region.

Provided herein also are methods to modulate expression of sequences of interest. Modulation of expression can be expression itself, an increase in expression, or a decrease in expression. Such a method can involve transforming a plant cell with, or growing a plant cell comprising, at least one recombinant nucleic acid construct. A recombinant nucleic acid construct can include a regulatory region as described above, e.g., comprising a nucleic acid having 80% or greater sequence identity to a regulatory region set forth in SEQ ID NOs:22-37, where the regulatory region is operably linked to a nucleic acid encoding a sequence of interest. In some cases, a recombinant nucleic acid construct can further include a nucleic acid encoding a regulatory protein as described above, e.g., comprising a polypeptide sequence having 80% or greater sequence identity to a polypeptide sequence set forth in SEQ ID NOs:2-13, SEQ ID NOs:15-19, SEQ ID NO:21, and the consensus sequences set forth in FIGS. 1-2. In other cases, the nucleic acid encoding the described regulatory protein is contained on a second recombinant nucleic acid construct. In either case, the regulatory region and the regulatory protein are associated, e.g., as shown in Table 4 (under Example 5 below) or as described herein (e.g., all orthologs of a regulatory protein are also considered to associate with the regulatory regions shown to associate with a given regulatory protein in Table 4, under Example 5 below). A plant cell is typically grown under conditions effective for the expression of the regulatory protein.

As will be recognized by those having ordinary skill in the art, knowledge of an associated regulatory region-regulatory protein pair can also be used to modulate expression of endogenous sequences of interest that are operably linked to endogenous regulatory regions. In such cases, a method of modulating expression of a sequence of interest includes transforming a plant cell that includes an endogenous regulatory region as described herein, with a recombinant nucleic acid construct comprising a nucleic acid encoding a regulatory protein as described herein, where the regulatory region and the regulatory protein are associated as indicated in Table 4 (under Example 5 below) and as described herein. Accordingly, an orthologous sequence and a polypeptide corresponding to the consensus sequence of a given regulatory protein would also be considered to be associated with the regulatory region shown in Table 4 (under Example 5 below) to be associated with the given regulatory protein. A method for expressing an endogenous sequence of interest can include growing such a plant cell under conditions effective for the expression of the regulatory protein. An endogenous sequence of interest can in certain cases be a nucleic acid encoding a polypeptide involved in alkaloid biosynthesis, such as an alkaloid biosynthesis enzyme or a regulatory protein involved in alkaloid biosynthesis.

In other cases, knowledge of an associated regulatory region-regulatory protein pair can be used to modulate expression of exogenous sequences of interest by endogenous regulatory proteins. Such a method can include transforming a plant cell that includes a nucleic acid encoding a regulatory protein as described herein, with a recombinant nucleic acid construct comprising a regulatory region described herein, where the regulatory region is operably linked to a sequence of interest, and where the regulatory region and the regulatory protein are associated as shown in Table 4 (under Example 5 below) and described herein. A method of expressing a sequence of interest can include growing such a plant cell under conditions effective for the expression of the endogenous regulatory protein.

Also provided are methods for producing one or more alkaloids. Such a method can include growing a plant cell that includes a nucleic acid encoding an exogenous regulatory protein as described herein and an endogenous regulatory region as described herein operably linked to a sequence of interest. The regulatory protein and regulatory region are associated, as described previously. A sequence of interest can encode a polypeptide involved in alkaloid biosynthesis. A plant cell can be from a plant capable of producing one or more alkaloids. The plant cell can be grown under conditions effective for the expression of the regulatory protein. The one or more alkaloids produced can be novel alkaloids, e.g., not normally produced in a wild-type plant cell.

Alternatively, a method for producing one or more alkaloids can include growing a plant cell that includes a nucleic acid encoding an endogenous regulatory protein as described herein and a nucleic acid including an exogenous regulatory region as described herein operably linked to a sequence of interest. A sequence of interest can encode a polypeptide involved in alkaloid biosynthesis. A plant cell can be grown under conditions effective for the expression of the regulatory protein. The one or more alkaloids produced can be novel alkaloids, e.g., not normally produced in a wild-type plant cell.

Provided herein also are methods for modulating (e.g., altering, increasing, or decreasing) the amounts of one or more alkaloids in a plant cell. The method can include growing a plant cell as described above, e.g., a plant cell that includes a nucleic acid encoding an endogenous or exogenous regulatory protein, where the regulatory protein associates with, respectively, an exogenous or endogenous regulatory region operably linked to a sequence of interest. In such cases, a sequence of interest can encode a polypeptide involved in alkaloid biosynthesis. Alternatively, a sequence of interest can result in a transcription product such as an antisense RNA or interfering RNA that affects alkaloid biosynthesis pathways, e.g., by modulating the steady-state level of mRNA transcripts available for translation that encode one or more alkaloid biosynthesis enzymes.

The invention will be further described in the following examples, which do not limit the scope of the invention described in the claims.

### EXAMPLES

### Example 1

### Generation of Arabidopsis Plants Containing Alkaloid Regulatory Region::Luciferase Constructs

T-DNA binary vector constructs were made using standard molecular biology techniques. A set of constructs were made that contained a luciferase coding sequence operably linked to one or two of the regulatory regions set forth in SEQ ID NO:22, SEQ ID NO:24, SEQ ID NOs:29-30, SEQ ID NO:31, SEQ ID NO:32, and SEQ ID NO:33. Each of these constructs also contained a marker gene conferring resistance to the herbicide Finale®.

Each construct was introduced into Arabidopsis ecotype Wassilewskija (WS) by the floral dip method essentially as described in Bechtold et al., C. R. Acad. Sci. Paris, 316:1194-1199 (1993). The presence of each reporter region::luciferase construct was verified by PCR. At least two independent events from each transformation were selected for further study; these events were referred to as Arabidopsis thaliana screening lines. T₁ (first generation transformant) seeds were germinated and allowed to self-pollinate. T₂ (second generation, progeny of self-pollinated T₁ plants) seeds were collected and a portion were germinated and allowed to self-pollinate. T₃ (third generation, progeny of self-pollinated T₂ plants) seeds were collected.

### Example 2

### Screening of Regulatory Proteins in Arabidopsis

T₂ or T₃ seeds of the Arabidopsis thaliana screening lines described in Example 1 were planted in soil comprising Sunshine LP5 Mix and Thermorock Vermiculite Medium #3 at a ratio of 60:40, respectively. The seeds were stratified at 4° C. for approximately two to three days. After stratification, the seeds were transferred to the greenhouse and covered with a plastic dome and tarp until most of the seeds had germinated. Plants were grown under long day conditions. Approximately seven to ten days post-germination, plants were sprayed with Finale® herbicide to confirm that the plants were transgenic. Between three to four weeks after germination, the plants were used for screening.

T-DNA binary vector constructs comprising a CaMV 35S constitutive promoter operably linked to one of the regulatory protein coding sequences listed in Table 4 (under Example 5 below) were made and transformed into Agrobacterium. One colony from each transformation was selected and maintained as a glycerol stock. Two days before the experiment commenced, each transformant was inoculated into 150 μL of YEB broth containing 100 μg/mL spectinomycin, 50 μg/mL rifampicin, and 20 μM acetosyringone; grown in an incubator-shaker at 28° C.; and harvested by centrifugation at 4,000 rpm for at least 25 minutes. The supernatant was discarded, and each pellet was resuspended in a solution of 10 mM MgCl; 10 mM MES, pH 5.7; and 150 μM acetosyringone to an optical density (OD₆₀₀) of approximately 0.05 to 0.1. Each suspension was transferred to a 1 mL syringe outfitted with a 30 gauge needle.

Plants were infected by mildly wounding the surface of a leaf using the tip of a syringe/needle containing a suspension of one of the Agrobacterium transformants. A small droplet of the Agrobacterium suspension was placed on the wound area after wounding. Each leaf was wounded approximately 10 times at different positions on the same leaf. Each leaf was wounded using one Agrobacterium transformant. The syringe needle preferably did not pierce through the leaf to increase the likelihood of Agrobacterium infection on the wounded site. Treated leaves were left attached to the mother plant for at least 5 days prior to analysis.

### Example 3

### Screening of Regulatory Proteins in Nicotiana

Stable Nicotiana tabacum screening lines, cultivar Samsun, were generated by transforming Nicotiana leaf explants with the T-DNA binary vector containing regulatory region and luciferase reporter construct as described in Example 1, following the transformation protocol essentially described by Rogers, S. G. et al., Methods in Enzymology 118:627 (1987). Leaf disks were cut from leaves of the screening lines using a paper puncher and were transiently infected with Agrobacterium clones prepared as described in Example 2. In addition, leaf disks from wild-type Nicotiana tabacum plants, cultivar SR1, were transiently infected with Agrobacterium containing a binary vector comprising a CaMV 35S constitutive promoter operably linked to a luciferase reporter coding sequence. These leaf disks were used as positive controls to indicate that the method of Agrobacterium infection was working. Some leaf disks from Nicotiana screening plants were transiently infected with Agrobacterium containing a binary construct of a CaMV 35S constitutive promoter operably linked to a GFP coding sequence. These leaf disks served as reference controls to indicate that the luciferase reporter activity in the treated disks was not merely a response to treatment with Agrobacterium.

Transient infection was performed by immersing the leaf disks in about 5 to 10 mL of a suspension of Agrobacterium culture, prepared as described in Example 2, for about 2 min. Treated leaf disks were briefly and quickly blot-dried in tissue paper and then transferred to a plate lined with paper towels sufficiently wet with 1× MS solution (adjusted to pH 5.7 with 1 N KOH and supplemented with 1 mg/L BAP and 0.25 mg/L NAA). The leaf disks were incubated in a growth chamber under long-day light/dark cycle at 22° C. for 5 days prior to analysis.

### Example 4

### Co-Infection Experiments in Nicotiana

In some cases, a mixture of two different Agrobacterium cultures were used in transient co-infection experiments in wild-type Nicotiana plants. One of the Agrobacterium cultures contained a vector comprising a regulatory region of interest operably linked to a luciferase reporter gene, and the other contained a vector that included the CaMV 35S constitutive promoter operably linked to a nucleotide sequence that coded for a regulatory factor of interest. The Agrobacterium culture and suspension were prepared as described in Example 2. The two different Agrobacterium suspensions were mixed to a final optical density (OD₆₀₀) of approximately 0.1 to 0.5. The mixture was loaded into a 1 mL syringe with a 30 gauge needle.

Depending on the size of a Nicotiana leaf, it can be divided arbitrarily into several sectors, with each sector accommodating one type of Agrobacterium mixture. Transient infection of a wild-type tobacco leaf sector was done by mildly wounding the surface of a leaf using the tip of a syringe/needle containing a mixture of Agrobacterium culture suspensions. A small droplet of the Agrobacterium suspension was placed on the wound area after wounding. Each leaf sector was wounded approximately 20 times at different positions within the same leaf sector. Treated Nicotiana leaves were left intact and attached to the mother plant for at least 5 days prior to analysis. A leaf sector treated with Agrobacterium that contained a binary construct including a CaMV 35S constitutive promoter operably linked to a GFP coding sequence was used as a reference control.

### Example 5

### Luciferase Assay and Results

Treated intact leaves from Examples 2 and 4, and leaf disks from Example 3, were collected five days after infection and placed in a square Petri dish. Each leaf was sprayed with 10 μM luciferin in 0.01% Triton X-100. Leaves were then incubated in the dark for at least a minute prior to imaging with a Night Owl™ CCD camera from Berthold Technology. The exposure time depended on the screening line being tested; in most cases the exposure time was between 2 to 5 minutes. Qualitative scoring of luciferase reporter activity from each infected leaf was done by visual inspection and comparison of images, taking into account the following criteria: (1) if the luminescence signal was higher in the treated leaf than in the 35S-GFP-treated reference control (considered the background activity of the regulatory region), and (2) if the #1 criterion occurred in at least two independent transformation events carrying the regulatory region-luciferase reporter construct. Results of the visual inspection were noted according to the rating system given in Table 3, and with respect to both the positive and negative controls.

TABLE 3
Luciferase activity scoring system

| Score | Score Comment |
|---|---|
| ++ | signal in the treated leaf is much stronger than in reference |
| | background |
| + | signal in the treated leaf is stronger than in reference background |
| +/− | weak signal but still relatively higher than reference background |
| − | no response |

Alkaloid regulatory region/regulatory protein combinations that resulted in a score of ±, + or ++ in both independent Arabidopsis transformation events were scored as having detectable luciferase reporter activity. Combinations that resulted in a score of ±, + or ++ in one independent Arabidopsis transformation event were also scored as having detectable reporter activity if similar ratings were observed in the Nicotiana experiment. Combinations (also referred to as associations herein) having detectable luciferase reporter activity are shown in Table 4, below.

TABLE 4
Combinations of regulatory regions and regulatory proteins producing
expression of a reporter gene operably linked to each regulatory region

| | Regulatory | Regulatory | Regulatory | |
|---|---|---|---|---|
| Regulatory Region | Protein | Protein | Protein | Screening |
| Construct | SEQ ID NO: | Gemini_ID | cDNA_ID | Organism |
| AtSS3-L-AtROX7-L | 2 | 5110B8 | 23461192 | Arabidopsis thaliana |
| | | | | and Tobacco |
| AtSS3-L-AtROX7-L | 15 | 5110C9 | 23660631 | Arabidopsis thaliana |
| | | | | and Tobacco |
| PsHMCOMT2-L | 15 | 5110C9 | 23660631 | Tobacco |
| PsROMT-L | 15 | 5110C9 | 23660631 | Tobacco |
| EcBBE-L-EcNMCH3-L | 21 | 552G1 | 23777863 | Arabidopsis thaliana |
| PsROMT-L | 21 | 552G1 | 23777863 | Tobacco |
| PsSAT-L | 21 | 552G1 | 23777863 | Tobacco |

Legend:
L = Luciferase
K = Kanamycin (neomycin phosphotransferase)
AtROX7 = Arabidopsis putative reticuline oxidase gene 7 promoter
EcNMCH3 = Eschscholzia californica N-methylcoclaurine 3′-hydroxylase gene promoter
AtSS3 = Arabidopsis putative strictosidine synthase gene 3 promoter
EcBBE = Eschscholzia californica berberine bridge enzyme promoter
PsHMCOMT2 = Papaver somniferum hydroxy N-methyl S-coclaurine 4-O-methyltransferase 2 gene promoter
PsROMT = Papaver somniferum (R,S)-reticuline 7-O-methyltransferase (PsROMT) gene promoter
PsSAT = Papaver somniferum (7S)-salutaridinol 7-O-acetyltransferase gene promoter

### Example 6

### Determination of Functional Homolog and/or Ortholog Sequences

A subject sequence was considered a functional homolog or ortholog of a query sequence if the subject and query sequences encoded proteins having a similar function and/or activity. A process known as Reciprocal BLAST (Rivera et al., Proc. Natl. Acad. Sci. USA, 95:6239-6244 (1998)) was used to identify potential functional homolog and/or ortholog sequences from databases consisting of all available public and proprietary peptide sequences, including NR from NCBI and peptide translations from Ceres clones.

Before starting a Reciprocal BLAST process, a specific query polypeptide was searched against all peptides from its source species using BLAST in order to identify, polypeptides having sequence identity of 80% or greater to the query polypeptide and an alignment length of 85% or greater along the shorter sequence in the alignment. The query polypeptide and any of the aforementioned identified polypeptides were designated as a cluster.

The main Reciprocal BLAST process consists of two rounds of BLAST searches; forward search and reverse search. In the forward search step, a query polypeptide sequence, “polypeptide A,” from source species SA was BLASTed against all protein sequences from a species of interest. Top hits were determined using an E-value cutoff of 10⁻⁵ and an identity cutoff of 35%. Among the top hits, the sequence having the lowest E-value was designated as the best hit, and considered a potential functional homolog or ortholog. Any other top hit that had a sequence identity of 80% or greater to the best hit or to the original query polypeptide was considered a potential functional homolog or ortholog as well. This process was repeated for all species of interest.

In the reverse search round, the top hits identified in the forward search from all species were BLASTed against all protein sequences from the source species SA. A top hit from the forward search that returned a polypeptide from the aforementioned cluster as its best hit was also considered as a potential functional homolog or ortholog.

Functional homologs and/or orthologs were identified by manual inspection of potential functional homolog and/or ortholog sequences. Representative functional homologs and/or orthologs for SEQ ID NO:2 and SEQ ID NO:15 are shown in FIGS. 1-2, respectively. The percent identities of functional homologs and/or orthologs to SEQ ID NO:2 and SEQ ID NO:15 are shown below in Tables 5 and 6, respectively.

TABLE 5
Percent identity to Ceres cDNA ID 23461192 (SEQ ID NO: 2)

| | | SEQ | | |
|---|---|---|---|---|
| | | ID | % | |
| Designation | Species | NO: | Identity | e-value |
| Public GI no. 11602842 | Arabidopsis | 3 | 98.95 | 0 |
| | thaliana | | | |
| Public GI no. 56384438 | Thellungiella | 4 | 91.58 | 0 |
| | halophila | | | |
| Public GI no. 9857294 | Vigna unguiculata | 5 | 74.18 | 0 |
| Public GI no. 5360186 | Prunus armeniaca | 6 | 72.53 | 0 |
| Public GI no. 38112202 | Vitis vinifera | 7 | 71.28 | 0 |
| Public GI no. 1370274 | Nicotiana | 8 | 69.94 | 0 |
| | plumbaginifolia | | | |
| Public GI no. 17402597 | Citrus unshiu | 9 | 69.53 | 0 |
| Public GI no. 1673406 | Capsicum annuum | 10 | 66.31 | 0 |
| Ceres CLONE ID no. | Triticum aestivum | 11 | 64.62 | 0 |
| 921919 | | | | |
| Public GI no. 1772985 | Lycopersicon | 12 | 69.03 | 0 |
| | esculentum | | | |
| Public GI no. 50900462 | Oryza sativa subsp. | 13 | 69.9 | 0 |
| | japonica | | | |

TABLE 6
Percent identity to Ceres cDNA ID 23660631 (SEQ ID NO: 15)

| | | SEQ ID | % | |
|---|---|---|---|---|
| Designation | Species | NO: | Identity | e-value |
| Ceres CLONE ID no. | Triticum aestivum | 16 | 74.2 | 2.40E−89 |
| 763471 | | | | |
| Ceres CLONE ID no. | Zea mays | 17 | 73.8 | 4.79E−84 |
| 218529 | | | | |
| Ceres CLONE ID no. | Glycine max | 18 | 72.9 | 3.70E−93 |
| 481192 | | | | |
| Ceres CLONE ID no. | Glycine max | 19 | 72.7 | 3.89E−98 |
| 517528 | | | | |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A plant cell comprising an exogenous nucleic acid, said exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, said nucleic acid operably linked to a regulatory region that modulates transcription of said regulatory protein in said plant cell.

2. The plant cell of claim 1, wherein said regulatory region is a tissue-preferential promoter.

3. The plant cell of claim 2, wherein said tissue-preferential promoter is a vascular tissue-preferential promoter or a poppy capsule-preferential promoter.

4. The plant cell of claim 1, wherein said plant cell further comprises an endogenous regulatory region that is associated with said regulatory protein.

5. The plant cell of claim 1, wherein said plant cell further comprises an exogenous regulatory region operably linked to a sequence of interest, wherein said exogenous regulatory region is associated with said regulatory protein, and wherein said exogenous regulatory region comprises a nucleic acid having 80% or greater sequence identity to a regulatory region set forth in SEQ ID NOs:22-37.

6. The plant cell of claim 1, wherein said regulatory protein modulates expression of an endogenous polypeptide involved in alkaloid biosynthesis in said cell.

7. The plant cell of any of claims 1-6, wherein said plant cell is capable of producing one or more alkaloids.

8. The plant cell of claim 7, wherein at least one of said one or more alkaloids is salutaridine, salutaridinol, salutaridinol acetate, thebaine, isothebaine, papaverine, narcotine, noscapine, narceine, hydrastine, oripavine, morphinone, morphine, codeine, codeinone, or neopinone.

9. The plant cell of claim 7, wherein said plant is a member of the Papaveraceae, Menispermaceae, Lauraceae, Euphorbiaceae, Berberidaceae, Leguminosae, Boraginaceae, Apocynaceae, Asclepiadaceae, Liliaceae, Gnetaceae, Erythroxylaceae, Convolvulaceae, Ranunculaeceae, Rubiaceae, Solanaceae, or Rutaceae families.

10. The plant cell of claim 7, wherein said plant is a member of the species Papaver bracteatum, Papaver orientale, Papaver setigerum, Papaver somniferum, Croton salutaris, Croton balsamifera, Sinomenium acutum, Stephania cepharantha, Stephania zippeliana, Litsea sebiferea, Alseodaphne perakensis, Cocculus laurifolius, Duguetia obovata, Rhizocarya racemifera, or Beilschmiedia oreophila.

11. The plant cell of claim 7, wherein said cell further comprises a nucleic acid encoding a second regulatory protein operably linked to a second regulatory region that modulates transcription of said second regulatory protein in said plant cell.

12. The plant cell of claim 5, wherein said sequence of interest comprises a coding sequence for a polypeptide involved in alkaloid biosynthesis.

13. The plant cell of claim 12, wherein said polypeptide is an alkaloid biosynthesis enzyme.

14. The plant cell of claim 12, wherein said polypeptide is a regulatory protein involved in alkaloid biosynthesis.

15. The plant cell of claim 4 or 5, wherein said regulatory protein-regulatory region association is effective for modulating the amount of at least one alkaloid compound in said cell.

16. A Papaveraceae plant comprising an exogenous nucleic acid, said exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, said nucleic acid operably linked to a regulatory region that modulates transcription of said regulatory protein in said plant cell.

17. A method of modulating the expression level of one or more genes in a plant cell, said method comprising transforming said plant cell with an isolated nucleic acid comprising a nucleotide sequence encoding a regulatory protein comprising a polypeptide sequence set forth in SEQ ID NOs:2-13, SEQ ID NOs: 15-19, SEQ ID NO:21, or a consensus sequence set forth in FIG. 1 or FIG. 2, wherein said nucleotide sequence is operably linked to a regulatory region that modulates transcription in said plant cell.

18. The method of claim 17, wherein said plant cell is a member of the Papaveraceae family.

19. The method of claim 17, wherein said one or more genes are involved in alkaloid biosynthesis.

20. A method of expressing a sequence of interest comprising: growing a plant cell comprising:
1) an exogenous nucleic acid comprising a regulatory region comprising a nucleic acid having 80% or greater sequence identity to a regulatory region set forth in SEQ ID NOs:22-37, wherein said regulatory region is operably linked to a sequence of interest; and
2) an exogenous nucleic acid comprising a nucleic acid encoding a regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2;
wherein said regulatory region and said regulatory protein are associated, and wherein said plant cell is grown under conditions effective for the expression of said regulatory protein.

21. A method of expressing an endogenous sequence of interest comprising growing a plant cell comprising an endogenous regulatory region operably linked to a sequence of interest, wherein said endogenous regulatory region comprises a nucleic acid having 80% or greater sequence identity to a regulatory region set forth in SEQ ID NOs:22-37, wherein said plant cell further comprises a nucleic acid encoding an exogenous regulatory protein, said exogenous regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, wherein said exogenous regulatory protein and said endogenous regulatory region are associated, and wherein said plant cell is grown under conditions effective for the expression of said exogenous regulatory protein.

22. A method of expressing an exogenous sequence of interest comprising growing a plant cell comprising an exogenous regulatory region operably linked to a sequence of interest, wherein said exogenous regulatory region comprises a nucleic acid having 80% or greater sequence identity to a regulatory region set forth in SEQ ID NOs:22-37, wherein said plant cell further comprises a nucleic acid encoding an endogenous regulatory protein, said endogenous regulatory protein comprising a polypeptide sequence having 80% or greater sequence identity to a polypeptide sequence set forth in SEQ ID NOs:2-13, SEQ ID NOs:15-19, SEQ ID NO:21, or a consensus sequence set forth in FIG. 1 or FIG. 2, wherein said regulatory region and said regulatory protein are associated, and wherein said plant cell is grown under conditions effective for the expression of said endogenous regulatory protein.

23. A method of producing one or more alkaloids in a plant cell comprising growing the plant cell of claim 4 under conditions effective for the expression of said regulatory protein, wherein said plant cell is capable of producing one or more alkaloids, and wherein said endogenous regulatory region is operably linked to a sequence of interest comprising a coding sequence for a polypeptide involved in alkaloid biosynthesis.

24. A method of producing one or more alkaloids in a plant cell comprising growing the plant cell of claim 5 under conditions effective for the expression of said regulatory protein, wherein said sequence of interest comprises a coding sequence for a polypeptide involved in alkaloid biosynthesis.

25. A method of modulating the level of one or more alkaloid compounds in a plant cell, said method comprising transforming said plant cell with an isolated nucleic acid comprising a nucleotide sequence encoding a regulatory protein comprising a polypeptide sequence set forth in SEQ ID NOs:2-13, SEQ ID NOs:15-19, SEQ ID NO:21, or a consensus sequence set forth in FIG. 1 or FIG. 2, wherein said nucleotide sequence is operably linked to a regulatory region that modulates transcription in said plant cell, and wherein a plant produced from said plant cell has a difference in the level of said alkaloid compared to the level of said alkaloid in a corresponding control plant that does not comprise said isolated nucleic acid.


SEQUENCE LISTING (122 sequences)

SEQ ID NO: 1 - DNA, 3346 bp, Arabidopsis thaliana
  misc_feature (1)..(3346): Ceres CDNA ID no. 23461192
  misc_feature (1)..(3346): Also Known As Ceres GEMINI ID 5110B8
atgggttcaactccgttttgctactctatcaatccatctccatcaaagcttgatttcacgaggacccatgtgtttagtcctgtttctaaacagttttacttagatttatcatcgttttccggaaaacccggaggagtatctgggtttaggagccgtcgagctttgctcggagtaaaggcggcgacggcgttagttgagaaggaggagaagagagaggcggtgacggagaagaagaagaaatcgagggttttagttgccggaggtggaatcggaggattggtgtttgctttagcggctaagaagaaaggattcgatgtgttagtgtttgagaaagatttgagtgctataagaggagaaggaaaatacagaggcccgattcaaatacagagcaacgctttagctgctttggaagctattgatattgaagttgctgaacaagttatggaagctgggtgtatcactggtgatcggattaacggtctcgttgatggtatctctggtacttggtgagtttccctgagatctcttgattgagtagccaatcagattattggaaacttgtggtgatatttttgtttgtgaatcaattaggtatgtaaagtttgatactttcactcctgcggcgtcacggggacttcctgtgactagagtaattagtagaatgactctgcagcagattctagcacgtgcggttggagaagatgtgattagaaacgagagtaatgttgttgattttgaagattctggagataaggtgatggatgattgatttttacttaaaagagactattgatataaggaaagaatccaatgtagctgtatgggtttgttggtttaggttactgtggtactcgagaatggtcaacgctatgaaggtgatctgcttgtgggtgcagatggcatttggtctaaggtaggttttaactgaatagagtataacaaacagctttcgttgttgagtatagaactgaaagattgtgccttttgatttttgtttgtaggtgagaaataatttgtttggccgtagtgaagctacttattcaggctacacttgttacacggggattgcagattttataccagcggatatcgagtctgttgggtatgttgcagtcagtctaaatctcatgcatatcttctctgattatgaatctttttctgatattgggtctgtttcattctctcttaaagctaccgggttttcttgggacacaaacagtactttgtttcttcggatgttggtggtggaaaaatgcaatggtatgcatttcacgaggaaccagctggtggggctgatgctccaaatggtaaatttctgcattgttttacttctttctatggatcgtatttagtattagttcacagaagcttcttttctaattgatttttcaggtatgaagaaaaggttgtttgaaatatttgacggttggtgcgacaatgtactcgacttgttgcatgcgactgaggaggaagccattctgagaagagatatttatgatagaagtcctggttttacttggggtaaagggcgtgttacgctgctcggggattctatccatgcgatgcagccaaatatgggtcaaggtggatgcatggccattgaggtatctatctaatatataaatccactcaaccttttgactgattttaaagtgttattgagggaaatttcttgatgtattttgcaggatagttttcaactagcattggagcttgatgaagcatggaaacagagtgttgaaacgactacacctgttgatgttgtttcctctttgaaaaggtaaaactaaaacacaatttgtttagtttgggcttctggtggatactaaacatgctcttgaattggttttagatatgaggaatctagaagactgagagtcgctattatccatgcaatggcgaggatggctgcaattatggcttccacttacaaagcatacttaggtgttgggcttggtcctctgtctgtgagtatctttatggttttcactatacatggaaatcattcttgtttgttcatttctaactcaattccaaatcttgtttctgcagttcttgacaaagtttagagtaccacatccaggaagagttggtggtagattcttcgttgacattgctatgccatcgatgcttgactgggtccttggaggtaacaggttagttctatctgtgagttttatctaaagagaacttgatttgacagacctgtaaagccaactcgtttacacgcttttgcagtgaaaaactccaaggaaggccacctagttgcagactcactgacaaagtatgtttctttctttctctatttctcactcaaaaaacttcactaaatgactaatatgagaatgtaaatatttctttcaaggccgatgaccggcttcgagagtggtttgaagatgacgatgctcttgaacgtactataaagggagagtatgtactcacaaatctgaattcgacatttagttccattatatttcttcacctacctaacaaaaaaacggccttgctctctatactgctttggtctgagtctgagtcttaatttctttgcagatggtatctaattccacacggcgacgattgttgcgtttcggaaacattatgtctaaccaaagatgaagatcaaccttgcatcgtcgggtatgaaacaactcaccattctgaattcctctcagatttatagtcaatggaaacttagaaactttgtattaaagatcttacagttttaaacttttgaaaactctgttgattttagaagcgaaccagatcaagattttcctggaatgcgcattgtgatcccttcgtctcaggtatataaactgtgtatctgatagtgtgacctgagatacttaaagagaactagtcaacgaacttaattatgttttggtttaggtttcgaagatgcatgctcgtgtgatttacaaagacggagctttcttcttgatggatcttcgaagcgaacacggaacctatgtgaccgagtaagtctatatggatactctgaccaggttattatatctgagtctttgaagtttctgatttgatatttgaatgttttctagtaacgaaggaagaagatatagagcaacaccgaattttcccgcgcggtttagatcgtccgacatcatcgagtttggttcagataagaaggtgagagacatctatcacccctaccaaatctcggtttttcttttaaccgaatcatttacaataaccggtcttggtttctgaaaattatccggtttggttttgtcaggcggcgtttagggtgaaagtaatcaggaaaactccgaaatcgacgaggaagaatgagagtaacaacgataaattacttcagacagcttga

SEQ ID NO: 2 - protein, 667 aa, Arabidopsis thaliana
  misc_feature (1)..(667): Ceres CDNA ID no. 23461192
  misc_feature (1)..(667): Also Known As Ceres GEMINI ID 5110B8
  misc_feature (208)..(244): Pfam Name Pyr_redox;
  misc_feature (365)..(397): Pfam Name FAD_binding_3;
  misc_feature (558)..(634): Pfam Name FHA; Pfam Description FHA domain
  misc_feature (82)..(112): Pfam Name Pyr_redox_2;
MGSTPFCYSINPSPSKLDFTRTHVFSPVSKQFYLDLSSFSGKPGGVSGFRSRRALLGVKAATALVEKEEKREAVTEKKKKSRVLVAGGGIGGLVFALAAKKKGFDVLVFEKDLSAIRGEGKYRGPIQIQSNALAALEAIDIEVAEQVMEAGCITGDRINGLVDGISGTWYVKFDTFTPAASRGLPVTRVISRMTLQQILARAVGEDVIRNESNVVDFEDSGDKVTVVLENGQRYEGDLLVGADGIWSKVRNNLFGRSEATYSGYTCYTGIADFIPADIESVGYRVFLGHKQYFVSSDVGGGKMQWYAFHEEPAGGADAPNGMKKRLFEIFDGWCDNVLDLLHATEEEAILRRDIYDRSPGFTWGKGRVTLLGDSIHAMQPNMGQGGCMAIEDSFQLALELDEAWKQSVETTTPVDVVSSLKRYEESRRLRVAIIHAMARMAAIMASTYKAYLGVGLGPLSFLTKFRVPHPGRVGGRFFVDIAMPSMLDWVLGGNSEKLQGRPPSCRLTDKADDRLREWFEDDDALERTIKGEWYLIPHGDDCCVSETLCLTKDEDQPCIVGSEPDQDFPGMRIVIPSSQVSKMHARVIYKDGAFFLMDLRSEHGTYVTDNEGRRYRATPNFPARFRSSDIIEFGSDKKAAFRVKVIRKTPKSTRKNESNNDKLLQTA

SEQ ID NO: 3 - protein, 667 aa, Arabidopsis thaliana
  misc_feature (1)..(667): Public GI no. 11602842
  misc_feature (1)..(667): Functional Homolog of  Ceres CDNA ID no. 23461192 at SEQ ID NO. 2 with e-value of 0.0 and percent identity of 98.95
MGSTPFCYSINPSPSKLDFTRTHVFSPVAKQFYLDLSSFSGRSGGGLSVFRSRKTLLGVKAATALVEKEEKREAVTEKKKSRVLVAGGGIGGLVFALAAKKKGFDVLVFEKDLSAIRGEGKYRGPIQIQSNALAALEAIDIEVAEQVMEAGCITGDRINGLVDGISGTWYVKFDTFTPAASRGLPVTRVISRMTLQQILARAVGEDVIRNESNVVDFEDSGDKVTVVLENGQRYEGDLLVGADGIWSKVRNNLFGRSEATYSGYTCYTGIADFIPADIESVGYRVFLGHKQYFVSSDVGGGKMQWYAFHEEPAGGADAPNGMKKRLFEIFDGWCDNVLDLLHATEEEAILRRDIYDRSPGFTWGKGRVTLLGDSIHAMQPNMGQGGCMAIEDSFQLALELDEAWKQSVETTTPVDVVSSLKRYEESRRLRVAIIHAMARMAAIMASTYKAYLGVGLGPLSFLTKFRVPHPGRVGGRFFVDIAMPSMLDWVLGGNSEKLQGRPPSCRLTDKADDRLREWFEDDDALERTIKGEWYLIPHGDDCCVSETLCLTKDEDQPCIVGSEPDQDFPGMRIVIPSSQVSKMHARVIYKDGAFFLMDLRSEHGTYVTDNEGRRYRATPNFPARFRSSDIIEFGSDKKAAFRVKVIRKTPKSTRKNESNNDKLLQTA

SEQ ID NO: 4 - protein, 666 aa, Thellungiella halophila
  misc_feature (1)..(666): Public GI no. 56384438
  misc_feature (1)..(666): Functional Homolog of  Ceres CDNA ID no. 23461192 at SEQ ID NO. 2 with e-value of 0.0 and percent identity of 91.58
  misc_feature (492)..(492): Xaa is any aa, unknown, or other
MGSTPFCYSINPSPSKLDFTRTHVFSPVAKQFYLDLSSCAGKSGGGLSGFRSRRALVGVRAATALVEEEKREIAKEKKKPRVLVAGGGIGGLVFALAAKKKGFDVLVFEKDLSAIRGEGQYRGPIQIQSNALAALEAIDIDVAEEVMEAGCITGDRINGLVDGVSGTWYVKFDTFTPAASRGLPVTRVISRMTLQQILARAVGEEVIRNESNVVDFEDSGDKVTVVLENGERYEGDLLVGADGIWSKVRNNLFGRSEATYSGYTCYTGIADFVPADIESVGYRVFLGHKQYFVSSDVGGGKMQWYAFHEEPAGGVDAPNGMKKRLFDIFEGWCDNVLDLLHATEEEAILRRDIYDRTPSFNWGKGRVTLLGDSIHAMQPNMGQGGCMAIEDSYQLALELEEAWERSVETNAPVDVVSSLRRYEESRRLRVAIIHGMARMAAIMASTYKAYLGVGLGPLSFLTKFRVPHPGRVGGRFFIDIAMPLMLNWVLGXNSEKLEGRPPSCRLTDKADDRLREWFEDDDALERTINGEWYLIPYGNECSVSETLCLTKDEDQPCIIGSEPDQDFPGMHIVIPAPQVSKMHARVTYKDGAFFLMDLRSEHGTYVTDNEGRRYRVTPNFPARFRSSDIIEFGSDKKAAFRVKVIRTTPKSTSKNKESNGKLLQAV

SEQ ID NO: 5 - protein, 612 aa, Vigna unguiculata
  misc_feature (1)..(612): Public GI no. 9857294
  misc_feature (1)..(612): Functional Homolog of  Ceres CDNA ID no. 23461192 at SEQ ID NO. 2 with e-value of 0.0 and percent identity of 74.18
MLVKGAVVEAPPSVSPSSQGGSGAASKKQLRVLVAGGGIGGLVFALAAKKKGFDVVVFEKDLSAIRGEGQYRGPIQIQSNALAALEAIDSEVAEEVMRVGCITGDRINGLVDGVSGSWYVKFDTFTPAVERGLPVTRVISRMVLQEILARAVGEDIIMNASNVVNFVDDGNKVTVELENGQKYEGDILVGADGIWSKVRKQLFGHKEAVYSGYTCYTGIADFVPADIETVGYRVFLGHKQYFVSSDVGAGKMQWYAFHKEPPGGVDGPNGKKERLLKIFEGWCDNAVDLILATEEDAILRRDIYDRIPTLTWGKGRVTLLGDSVHAMQPNMGQGGCMAIEDSYQLALELDNAWEQSVKSGSPIDIDSSLRSYERERKLRVAIIHGMARMAALMASTYKAYLGVGLGPLEFLTKFRIPHPGRVGGRFFVDIMMPSMLSWVLGGNSSKLEGRPLSCRLSDKANDQLRQWFEDDEALERAINGEWILIPHGDGTSLSKPIVLSRNEMKPFIIGSAPAEDHPGTSVTIPSPQVSPRHARINYKDGAFFLIDLRSEHGTWIIDNEGKQYRVPPNYPARIRPSEAIQFGSEKVSFRVKVTRSVPRISENERPLTLQEA

SEQ ID NO: 6 - protein, 661 aa, Prunus armeniaca
  misc_feature (1)..(661): Public GI no. 5360186
  misc_feature (1)..(661): Functional Homolog of  Ceres CDNA ID no. 23461192 at SEQ ID NO. 2 with e-value of 0.0 and percent identity of 72.53
MASTLFYNSMNLSAAVFSRTHFPIPINKDFPLEFSPCIHTDYHLRSRTRSGQKKCLTEVRATVASPTEVPSAPASTQPKKLRILVAGGGIGGLVFALAAKKKGFDVVVFEKDLSAVRGEGQYRGPIQIQSNALAALEAIDMDVAEEVMRVGCVTGDRINGLVDGVSGTWYVKFDTFTPAVERGLPVTRVISRIALQQILARAVGEEIIINDSNVVNFEDLGDKVNVILENGQRYEGDMLVGADGIWSKVRKNLFGLNEAVYSGYTCYTGIADFVPADINSVGYRVFLGHKQYFVSSDVGGGKMQWYAFHKESPGGVDSPNGKKERLLKIFEGWCDNVIDLLLATEEDAILRRDIYDRTPILTWGKGHVTLLGDSVHAMQPNMGQGGCMAIEDGYQLALELDKAWKKSSETGTPVDVASSLRSYENSRRLRVAIIHGMARMAALMASTYKAYLGVGLGPLSFLTKFRIPHPGRVGGRVFIDKAMPLMLSWVLGGNSSKLEGRSPSCRLSDKASDQLRNWFEDDDALERAIDGEWYLIPCGQDNDASQLICLNRDEKNPCIIGSAPHGDVSGISIAIPKPQVSEMHARISYKDGAFYLTDLRSEHGTWIADIEGKRYRVPPNFPARFRPSDAIEIGSQKVAFRVKVMKSSPGSVEKEGILQAA

SEQ ID NO: 7 - protein, 658 aa, Vitis vinifera
  misc_feature (1)..(658): Public GI no. 38112202
  misc_feature (1)..(658): Functional Homolog of  Ceres CDNA ID no. 23461192 at SEQ ID NO. 2 with e-value of 0.0 and percent identity of 71.28
MASAVFYSSVQPSIFSRTHIPIPISKDSFEEFGHSINYKHYFRSNPCGQKKRVAQVKATLAEATPAPSAPSLPSKRVRILVAGGGIGGLVLALAAKKKGFDVVVFEKDMSAIRGEGQFRGPIQIQSNALAALEAVDMEVAEEVMRAGCITGDRINGLVDGVSGDWYVKFDTFTPAAERGLPVTRVISRMTLQQILARAVGEDIIMNGSNVVDFEDDGNKVTVILENGQRYEGDLLIGADGIWSKVRKSLFGPKEATYSGYTCYTGIADFVPADIDSVGYRVFLGHKQYFVSSDVGAGKMQWYAFYNEPAGGVDGPEGKKERLLKIFGGWCDNVIDLILATDEEAILRRDIYDRTPTFTWGRGRVTLLGDSVHAMQPNMGQGGCMAIEDSYQLAMELDKAWEQSIKSGTPIDVVSCLKSYEKARRIRVAVIHGMARMAAIMASTYKAYLGVGLGPLSFLTKLRIPHPGRVGGRFFIDIAMPLMLSWVLGGNSSKLEGRPPSCRLSDKASDQLRRWFEDDDALERAIGGEWFLLPSGESGLQPICLSKDENKPCIIGSVSHTDFPGISTVIPSPKVSKMHARISCKDGAFFLTDLQSEHGTWITDNVGRRQRVSPNFPTRFHPSEVIDFGSEKASFRVKVVRTPPDNAAKDEESKLFQAV

SEQ ID NO: 8 - protein, 663 aa, Nicotiana plumbaginifolia
  misc_feature (1)..(663): Public GI no. 1370274
  misc_feature (1)..(663): Functional Homolog of  Ceres CDNA ID no. 23461192 at SEQ ID NO. 2 with e-value of 0.0 and percent identity of 69.94
MYSTVFYTSVHPSTSAFSRKQLPLLISKDFPTELYHSLPCSRSLENGQIKKVKGVVKATIAEAPATIPPTDLKKVPQKKLKVLVAGGGIGGLVFALAAKKRGFDVLVFERDLSAIRGEGQYRGPIQIQSNALAALEAIDMDVAEDIMNAGCITGQRINGLVDGVSGNWYCKFDTFTPAVERGLPVTRVISRMTLQQNLARAVGEDIIMNESNVVNFEDDGEKVTVTLEDGQQYTGDLLVGADGIRSKVRTNLFGPSDVTYSGYTCYTGIADFVPADIETVGYRVFLGHKQYFVSSDVGGGKMQWYAFHNEPAGGVDDPNGKKARLLKIFEGWCDNVIDLLVATDEDAILRRDIYDRPPTFSWGKGRVTLLGDSVHAMQPNLGQGGCMAIEDSYQLALELDKALSRSAESGTPVDIISSLRSYESSRKLRVGVIHGLARMAAIMASTYKAYLGVGLGPLSFLTKFRIPHPGRVGGRFFIDLGMPLMLSWVLGGNGEKLEGRIQHCRLSEKANDQLRNWFEDDDALERATDAEWLLLPAGNSNAALETLVLSRDENMPCNIGSVSHANIPGKSVVIPLPQVSEMHARISYKGGAFFVTDLRSEHGTWITDNEGRRYRASPNFPTRFHPSDIIEFGSDKKAAFRVKVMKFPPKTAAKEERQAVGAA

SEQ ID NO: 9 - protein, 664 aa, Citrus unshiu
  misc_feature (1)..(664): Public GI no. 17402597
  misc_feature (1)..(664): Functional Homolog of  Ceres CDNA ID no. 23461192 at SEQ ID NO. 2 with e-value of 0.0 and percent identity of 69.53
MVSSMFYNSVNLSTAVFSRTHFPVPVYKHSCIEFSRYDHCINYKFRTGTSGQSKNPTQMKAAVAESPTNNSDSENKKLRILVAGGGIGGLVFALAAKRKGFEVLVFEKDMSAIRGEGQYRGPIQIQSNALAALEAIDLDVAEEVMRAGCVTGDRINGLVDGISGSWYIKFDTFTPAAEKGLPVTRVISRMTLQQILAKAVGDEIILNESNVIDFKDHGDKVSVVLENGQCYAGDLLIGADGIWSKVRKNLFGPQEAIYSGYTCYTGIADFVPADIESVGYRVFLGHKQYFVSSDVGAGKMQWYAFHKEPAGGVDDPEGKKERLLKIFEGWCDNVVDLILATDEEAILRRDIYDRTPIFTWGRGRVTLLGDSVHAMQPNLGQGGCMAIEDGYQLAVELEKACKKSNESKTPIDIVSALKSYERARRLRVAVIHGLARSAAVMASTYKAYLGVGLGPLSFLTKFRIPHPGRVGGRFFIDLAMPLMLSWVLGGNSSKLEGRSPCCKLSDKASDNLRTWFRDDDALERAMNGEWFLVPSGSENVVSQPIYLSGSHENEPYLIGSESHEDFPRTSIVIPSAQVSKMHARISYKDGAFYLIDLQSEHGTYVTDNEGRRYRVSSNFPARFRPSDTIEFGSDKKAIFRVKVIGTPPNNNSERKEAGEILQAV

SEQ ID NO: 10 - protein, 660 aa, Capsicum annuum
  misc_feature (1)..(660): Public GI no. 1673406
  misc_feature (1)..(660): Functional Homolog of  Ceres CDNA ID no. 23461192 at SEQ ID NO. 2 with e-value of 0.0 and percent identity of 66.31
MYASSARDGIPGKWCNARRKQLPLLISKDFPAELYHSLPCKSLENGHIKKVKGVKATLAEAPATPTEKSNSEVPQKKLKVLVAGGGIGGLVFALAGKKRGFDVLVFERDISAIRGEGQYRGPIQIQSNALAALEAIDMDVAEEIMNAGCITGQRINGLVDGISGNWYCKFDTFTPAVERGLPVTRVISRMTLQQILARLQGEDVIMNESHVVNFADDGETVTVNPELCQQYTGDLLVGADGIRSKVRTNLFGPSELTYSGYTCYTGIADFVPADIDTAGYRVFLGHKQYFVSSDVGGGKMQWYAFHNEPAGGVDAPNGKKERLLKIFGGWCDNVIDLSVATDEDAILRRDIYDRPPTFSWGKGRVTLLGDSVHAMQPNLGQGGCMAIEDSYQLALELEKAWSRSAESGSPMDVISSLRSYESARKLRVGVIHGLARMAAIMASAYKAYLGVGLGPLSFITKFRIPHPGRVGGRFFIDLGMPLMLSWVLGGNGEKLEGRIQHCRLSEKANDQLRNWFEDDDALERATDAEWLLLPAGNSNAALETLVLSRDENMPCTIGSVSHANIPGKSVVIPLSQVSDMHARISYNGGAFLGTAFRSDHGTWFIDNEGRRYRVSPNFPMRFHSSDVIVFGSDKAAFRIKAMKFAPKTAAKEDRQAVGAA

SEQ ID NO: 11 - protein, 651 aa, Triticum aestivum
  misc_feature (1)..(651): Ceres CLONE ID no. 921919
  misc_feature (1)..(651): Functional Homolog of  Ceres CDNA ID no. 23461192 at SEQ ID NO. 2 with e-value of 0.0 and percent identity of 64.62
MPCSTCTRVSPTAISATSSSSRTVLRILPFRSPRGPYGRGLCLPGSIRCTAEVVDAAMHAPKPPEPKPRVLVAGGGIGGLTFALAARRKGFQVLVLEKDMSAVRGEGKYRGPIQLQSNALAVLEAVDMPAADQIMDAGCITGDRVNGIVDGISGSWYIKFDTFTPAADRGLPVTRVISRMTLQQILARAVGDDAIMNDCHVVDFTDNGNKVTAILEDGRKFEGDLLVGADGIWSKVRKSLFGETDASYSEYTCYTGIADFVPPDIDTVGYRVFLGHKQYFVSSDVGGGKMQWYAFHKEPAGGTYPENGKKKRLLEIFSGWCDNVIDLLNATEEEAILRRDIYDRPPTINWGKGRVTLLGDSVHAMQPNLGQGGCMAIEDGYQLAVELEKAWEESVKSRTPVDVISSLRSYEKERKLRVAIIHGLARMAAIMATTYRPYLGVGLGPLSFLTKLRIPHPGRVGGRFFIKVGMPLMLSWILGGNSSKLEGRPLSCRLSDKASNQLGRWFQDDDALEQAMGGEWYLFPMSSGDDSALQPIRLIRDEQRTLSIGSKPDPSNSDSSLSFPLPQVSEIHATITCKNKGFYLTDLGSEHGTWFNDNEGRRYRLPPNFPVRFHPSDAIEFGSDKKAMFRVKVLSTLPYDSARGGEVLQAA

SEQ ID NO: 12 - protein, 669 aa, Lycopersicon esculentum
  misc_feature (1)..(669): Public GI no. 1772985
  misc_feature (1)..(669): Functional Homolog of  Ceres CDNA ID no. 23461192 at SEQ ID NO. 2 with e-value of 0.0 and percent identity of 69.03
MYSTVFYTSVHPSTSVLSRKQLPLLISKDFSAELYHSLPCRSLENGHINKVKGVKVKATIAEAPVTPTEKTDSGANGDLKVPQKKLKVLVAGGGIGGLVFALAAKKRGFDVLVFERDLSAIRGEGQYRGPIQIQSNALAALEAIDLDVAEDIMNAGCITGQRINGLVDGISGNWYCKFDTFTPAVERGLPVTRVISRMTLQQILARAVGEEIIMNESNVVDFEDDGEKVTVVLENGQRFTGDLLVGADGIRSKVRTNLFGPSEATYSGYTCYTGIADFVPADIDTVGYRVFLGHKQYFVSSDVGGGKMQWYAFYNEPAGGADAPNGKKERLLKIFGGWCDNVIDLLVATDEDAILRRDIYDRPPTFSWGRGRVTLLGDSVHAMQPNLGQGGCMAIEDSYQLALELEKACSRSAEFGSPVDIISSLRSYESARKLRVGVIHGLARMAAIMASTYKAYLGVGLGPLSFLTQYRIPHPGRVGGRVFIDLGMPLMLSWVLGGNGDKLEGRIKHCRLSEKANDQLRKWFEDDDALERATDAEWLLLPAGNGSSGLEAIVLSRDEDVPCTVGSISHTNIPGKSIVLPLPQVSEMHARISCKDGAFFVTDLRSEHGTWVTDNEGRRYRTSPNFPTRFHPSDVIEFGSDKAAFRVKAMKFPLKTSERKEEREAVEAA

SEQ ID NO: 13 - protein, 652 aa, Oryza sativa subsp. japonica
  misc_feature (1)..(652): Public GI no. 50900462
  misc_feature (1)..(652): Functional Homolog of  Ceres CDNA ID no. 23461192 at SEQ ID NO. 2 with e-value of 0.0 and percent identity of 69.9
MAPLPSSSSTRATSLVAIPGASCPHGQAQRLLAFRPPRRGSQWRGLCVSRGRHGATVAMAMPAAQAAGRRARVLVAGGGIGGLVFALAAKRKGFEVVVLERDMSAVRGEGKYRGPIQLQSNALAVLEAVDAGAADQVMDAGCITGNRVNGIVDGVSGSWYIKFDTFTPAAERGLPVTRVISRMTLQQILARAVGDDAILNDSHVVDFIDDGNKVTAILEDGRKFEGDLLVGADGIWSKVRKVLFGQSEATYSEYTCYTGIADFVPPDIDTVGYRVFLGHKQYFVSSDVGAGKMQWYAFHKEPAGGTDPENGKNKRLLEIFNGWCDNVVDLINATDEEAILRRDIYDRPPTFNWGKGRVTLLGDSVHAMQPNLGQGGCMAIEDGYQLAVELEKSWQESAKSGTPMDIVSSLRRYEKERILRVSVIHGLARMAAIMATTYRPYLGVGLGPLSFLTKLRIPHPGRVGGRFFIKYGMPLMLSWVLGGNSTKLEGRPLSCRLSDKANDQLRRWFEDDDALEQAMGGEWYLLPTSSGDSQPIRLIRDEKKSLSIGSRSDPSNSTASLALPLPQISENHATITCKNKAFYVTDNGSEHGTWITDNEGRRYRVPPNFPVRFHPSDAIEFGSDKKAVFRVKVLSTLPYESARGGPQILQAA

SEQ ID NO: 14 - DNA, 2330 bp, Arabidopsis thaliana
  misc_feature (1)..(2330): Ceres CDNA ID no. 23660631
  misc_feature (1)..(2330): Also Known As Ceres GEMINI ID 5110C9
atggctatggcctctgcttctggttctgctctttgcttcactgatgcttctagctctcttgctttaagacgcgattgtggagctctttgcttgcctcctaggacagttacgtttggatttgtcgataaacctcttgtcaatctcgagcgattaaggtttttgttttttggctatctcttgatgccttcttgagaatttgaattcgagcttcttttggattgctttttcgattgtgatcttagttttgtgctgattcgagtttcatgaagtaaatactgtttccgaaacaaattcttttgtagtgaattttctccacagatctctaaatccttaaaaagcatctgatacctttttttaacgattatcagaattttgtgttccgagttgctaatctttttaaaatgtccaaatgattttattgctggttataaattcgtatcttttgctggaattcgattcaaagcacagattgttttgagctgatcttctacggattcaaatacttcaattcaattcttttttttttttttcttcagattatccactttgaagattagagcatcaaatgctactgctgtggaggtatgaaaatgaaatgattacacaaggacactagtaatttgaatagactttttaaagtttatatgtttttgtttttgattttgaagaatggaaagcaagaaggaagtgctgctgattctgataaagttccaactccagttgttataatcgaccaagattctgaccctgatgcaaccgttcttgaagttacattcggagatcgtcttggagctttacttgacactgtaaggaaggataccttcttcttactcacttgttgctagctctgcataatcgaccaacattcacaaaagcttcttacttcctttcctgcttttttgactgcttcttgatctgatcaactttgcccttctagatgaatgcgctcaaaaacttgggattgaatgtcgtcaaggcaaatgtttaccttgattcttccggcaagcacaacaaatttgccattactagagcgtatgtcttttcatatcatcaaagagtattttgagctccaagagtcgtttagatagttaatgttgctttgaatcgattgactctgtcaaatagttgactttagattgagttttgagctgactgtaattgttgttacaacagagatagtggaagaaaagtagaagaccctgaattgcttgaggccattcgtctcacagtcataaacaatctgcttgagtttcatcctgtaactacttcacatcctaatatcctctctttctatttttcctatatcttgcataattttttaactcacacattgcttctcgatgatttggatcaggaatcaagttctcagttggcaatgggagcagcttttggtgttcttcctccaactgaaccggtaaaaaccttaaactgtgatgctctttttataaccttatccacttcttttgtattgcactaattcttcaagtagttagcatcctcgaacataaaggtcaagactgcttgtcgtttcctcggtcacgtacagataagaaacaataatagagaaaacaaagacttaactaataatcaacatatttggatttggcgggtcaatgtagagtacaagtattaaacttcattatagtattaatcactttgatgaagaagggaatacccacgcaacttcaaagtagcttcaaaattcattttccttcttctgtattttggattgggatgttgacagattgacgtggacatagcaacacatataaccattgaagatgatggaccagaccgcaggtaatgtatacccataccttatctcaactctagaaccttatccaaatgcttttattcttatgatatatgatctgatcattgatccataattatttgttgatgatgcagtttactcttcatagaatcagcggatagacctggactattagttgagctggtgaagatcatttcagatatcagcgtcgctgttgaatctggagaattcgacaccgaggtatctaataatcctgttttttaatgtctaaaacatataccctccgctataatatcgctctgtttctcatgttgcttcttcttgttgtatgtgaaacaaaaagggtttgttggctaaggtgaagtttcatgtaagctacaggaacaaagccctaatcaagcctctccagcaggttaaaattctgaaattagacttttctctttgataagttgaaatgtttttactgaaatgaaatcaaattttgtatttgtaggtccttgctaatagtctgaggtacttcttgaggcgtccatcaactgacgagtcaagtttctga

SEQ ID NO: 15 - protein, 290 aa, Arabidopsis thaliana
  misc_feature (1)..(290): Ceres CDNA ID no. 23660631
  misc_feature (1)..(290): Also Known As Ceres GEMINI ID 5110C9
  misc_feature (212)..(241): Pfam Name ACT; Pfam Description ACT domain
MAMASASGSALCFTDASSSLALRRDCGALCLPPRTVTFGFVDKPLVNLERLRLSTLKIRASNATAVENGKQEGSAADSDKVPTPVVIIDQDSDPDATVLEVTFGDRLGALLDTMNALKNLGLNVVKANVYLDSSGKHNKFAITRADSGRKVEDPELLEAIRLTVINNLLEFHPESSSQLAMGAAFGVLPPTEPIDVDIATHITIEDDGPDRSLLFIESADRPGLLVELVKIISDISVAVESGEFDTEGLLAKVKFHVSYRNKALIKPLQQVLANSLRYFLRRPSTDESSF

SEQ ID NO: 16 - protein, 278 aa, Triticum aestivum
  misc_feature (1)..(278): Ceres CLONE ID no. 763471
  misc_feature (1)..(278): Functional Homolog of  Ceres CDNA ID no. 23660631 at SEQ ID NO. 15with e-value of 2.40E-89 and percent identity of 74.2
MAVAVAAGTLRTCSGVFPAASGNHPLAGWRPLAPAAPAKLRLLSPALRVPRAASPAAVENGSSSNSNTVPTPKVIIDQDSDPDATIVEVTLGDRLGDLLDTMSALRNLGLNVVKASVCLDSSGKHNKFSITKSSTGRKIDDPELLEAVRLTIINNMLEYHPEASSQLAMGATFGLEPPTEVVDVDIATHIEIYDDGPERSLLVVESADRPGLLVDLVKIIADINITVQSGEFDTEGLLAKAKFHVSYRGRPLIKALQQVLANSLRYFLRRPTTEDASF

SEQ ID NO: 17 - protein, 277 aa, Zea mays
  misc_feature (1)..(277): Ceres CLONE ID no. 218529
  misc_feature (1)..(277): Functional Homolog of  Ceres CDNA ID no. 23660631 at SEQ ID NO. 15with e-value of 4.79E-84 and percent identity of 73.8
MAEMTVTVAAAKSTLRTCPGARRAAVLGRWMPLAPAAPANLKLFSPAVRVPRATSPAAVEDGSNTDIVPIPKVIIDQDSDPDATIVEITLGDRLGDLLDTMSALKNLGLNVVKASVCLDSTGKHIKFAITRAFTGRKIDDPELLEAVRLTIINNMIQYHPESSSQLAMGATFGPEAPTEEVDVDIATHIDIYDDGPERSLLVVETADRPGLLVDLVKIISDISINVQSGEFDTEGLLAKAKFHVSYRGRTLTEALQQVLSNSLRYFLRRPTTEDASF

SEQ ID NO: 18 - protein, 289 aa, Glycine max
  misc_feature (1)..(289): Ceres CLONE ID no. 481192
  misc_feature (1)..(289): Functional Homolog of  Ceres CDNA ID no. 23660631 at SEQ ID NO. 15with e-value of 3.70E-93 and percent identity of 72.9
MAVAMAACSLGLHLSADNTLRPFEKTTILKALSISDATKPCYISHKTRLSSSSSGITMIPRATTVIGTVEDGNQGEADTIPTPVVIIDQDSDPDATVVEITFGDRLGALLDTMNALKNLGLNVVKANVFLDSSGKHNKFSITKADSGRKVEDPELLEAIRLTILNNMIQYHPESSAQLALGAAFGLVPPKEQVDVEIATQITISDDGPKRSLLYVETADRPGLLVDLVKTITDINIAVESGEFDTEGLLAKAKFHVNYKDKALIKPLQLVLVNSLRYFLRRPETEESSF

SEQ ID NO: 19 - protein, 291 aa, Glycine max
  misc_feature (1)..(291): Ceres CLONE ID no. 517528
  misc_feature (1)..(291): Functional Homolog of  Ceres CDNA ID no. 23660631 at SEQ ID NO. 15with e-value of 3.89E-98 and percent identity of 72.7
MTLAMAVWSAGLHFSAARSSLRPLEKTICTAPFLKASSGFAATKPFCILNTTRLSYSGTTIIPRAAPVTDVKDGNQGETDTIPTPVVIIDQDSDPDATVVEITFGDRLGALLDTMNALKNLGLNVVKANVFLDSSGKHNKFSITKADTGRKVEEPELLEAIRLTIINNLIQYHPESSSQLALGAAFGLLPPKEQVDVDIATHINISDDGPDRSMLYVETADRPGLLVDLVKIITDINIAVESGEFDTEGLLAKAKFHVSYNGKAISKPLQQVLANSLRYFLRRPTTEESSF

SEQ ID NO: 20 - DNA, 1290 bp, Zea mays
  misc_feature (1)..(1290): Ceres CDNA ID no. 23777863
  misc_feature (1)..(1290): Also Known As Ceres GEMINI ID 552G1
  misc_feature (1)..(1290): Also Known As Ceres CLONE ID 304947
attttggagcccgatttattctctgtaggctggttcttttttagcccagtttgcctggaagaacagagatacgtggatttgatccaccaccagctcgagtttttgcttggcattgccctggagttggatgcattcaagtatttgcaaatagttgcttcatatggaggatgcacaggtgcaaacaatcttgataagaatggtaaactagactgaggagatttcgagtgggtgagcgcggcaacgacatgttgacaggagacggcgggaagcgctcgacaagctaattaacttgaaaggaagtgtcggggtcttctgtcgggtctgaccgcttttgaacactgagtcaccggttactgtcgagcaggagaggatcacggtcaagtcagtcgggattaagaaagagttcgatgctgatagggtctttgggcaggagtatgcacaagccaggaaaacatgcttcctgctgagtttgaaattaccgattgttgccatccttgtgattcggacaaggaaaatgttcttgctattcaagttatgagatggagtgatggctcttatcttgaagatcaggatcactggcggttgtctggtagcctcagtgataacttctttgcgatcattgtgccgactgagtacgattgtttaacggccagcactaggaagaaggaaattgttgatgtcatagttaaggctttcaagagcacatgagtatgaacctgaggtggcttgctggcgtacatggagtggtgacagattgcatgctggtagttgatgaaactggtgggatatttgagaaagatgacgagggaatgcctattcgaggtactggaggagttaggaatatccagccaaatggggacttgctagcaataaccccctcaatttatgtcacagtgttgcaaaaaattgggcggctacgtgactctagggtggaatttagatctactgtaagcagtgggaaaagtaggctatttcctgtagaggttctatttaataatgaaaaatggaataggcattgaaaaaacaagagatgaagctcaagctcaagatgctgagaaaactctccaaaatttaaagagggatgaatttgcatgagaggttatgaactgcaggcagagtgagtgatgggagtgtttgcatattaaagtgcactgcacgatggaggattaaggtgtttgtggatgttgagcgtttacttcagcttattcaatcttgtggcatttgtaatgttatccctgtaagaagatstccaatgtgttttgcaagatggacacgtccatcacgtctcgc

SEQ ID NO: 21 - protein, 84 aa, Zea mays
  misc_feature (1)..(84): Ceres CDNA ID no. 23777863
  misc_feature (1)..(84): Also Known As Ceres GEMINI ID 552G1
  misc_feature (1)..(84): Also Known As Ceres CLONE ID 304947
  misc_feature (2)..(46): Pfam Name Glyco_hydro_2_N;
  misc_feature (47)..(83): Pfam Name Myosin_tail_2; Pfam Description Myosin tail
MLPAEFEITDCCHPCDSDKENVLAIQVMRWSDGSYLEDQDHWRLSGSLSDNFFAIIVPTEYDCLTASTRKKEIVDVIVKAFKST

SEQ ID NO: 22 - DNA, 976 bp, Arabidopsis thaliana
  misc_feature (1)..(976): Ceres Promoter AtSS3
aaggtctgtttacagttattttaaccataataatatcccctcaaatgtcctgcttgttacgtgggtgacacaattgttcggtgattccttgaataggctactactacttattgtttcatattttcgaagagattatctctagttcttaaggctttggttttctttgttttcaccctttgttttacttcataaagacgaaaaacaagaaaaattaagtatctatactaaaaaaaaatatgatctataattcgatatgtacacacgtttatacattgacttttatacaacacgttagtagatagaaccgtcattcttatagtatcaatatataaactataaagtaagatactagaatgcggccgatttatataggtacatccattttgaccattttaatgtcataagatctcgaaggtgtgaaatgtgaatggtggtgattaaaaaaacaagagtgtgcggtgtcgtacggtgagacggtttattttgcactatcatcatatgtgattttcgtgcacgctttagagaaaaaaaaattccacaatccacactatacatatatagaccataataatattttatgcatggtacaaaatgcagaagcaatgcaaatcaaattgctttccatttccgctaccgcatttactagtgaatcaagaaaagtaaatgtcaaaacatttaatgaaatgttcctaagataatgtgtagtatcttttcatcaatctacttctaattaatcttaaaactaacgtaatttaggcgtatttgtttcttgatttatattcaatattaattactaattttagtttgatttgtttctataaatactttgtctattgtcaactttagaaaaaaaaaaaaaaaaagatgtcttaatataaggaaatttcaaaaaaggtaacattccaaaaataaatctataaatatacatacatacatacagataagagatagataaaaagaacacaaccaaagaaac

SEQ ID NO: 23 - DNA, 971 bp, Arabidopsis thaliana
  misc_feature (1)..(971): Ceres Promoter AtBBE2
ggttcgtcaatattgtcgaggtaataaagcgtagggtataatgtatgtgtagattttttgattgcctagtcaaagggtaaccggttaacaaggaagattattagttactacaattctaggacaggagagaatgtatttaggcttcacgccacaactactgaagatagtggtgtcgctatctttagatgtgataaacataaagtcgtgtcggtatccttgaaaagttgaaattagtgcgtattagcattgtttatccctaaaggcacatatatattgttccttactttcaaacgacatatatttcttcagaaccttttgctataatatgtctgaataatcattacaatatagtatatacacacaaaacttattgatactatgcattctctttggctatgttggaaaattcacttgcatgacttgtataaacttctaagttatgcatgcgttttaaataatcaagttactactataaattaagatacaggtatgtatagcaattaattaaaaactaattaatacagtttagtaatgaataatgatataaacattatacagtacagtgtcgctgtttcagtaatgatataatattcacatcagatttacatgtaaagattcagatcatagcaaattagcaatgaagtaaaggacaatgcaagaaattgaccaaattacttgaatattctaataattcatttcttttctttttttggttggcgaaacatatatatttggttacgttatatgcttaagggatatactgcttatttttcaagtaatcaaaatataaagatagaattatagtaaggagataaaagttgagattttagtaatagaataaaaatagtaatgaagtaatatttacaccaactttacgtgtaagccatccccacacacacatcaacctcctatataaacctaaacacatacctcaaacaaccaaaagcaaaaacctcatcgacataaaaaga

SEQ ID NO: 24 - DNA, 999 bp, Arabidopsis thaliana
  misc_feature (1)..(999): Ceres Promoter AtROX7
aactaattaggtcgttaattgtccaagggtttttcatagttgatatagttctgttcaaatatagccatccttaatcgattcatgggatcgtaaattactacttcgagtgttgtaaaaaaaaatgaaacttctacattacaaactcgaatttaatgcatctggagtgatactataaaagtagggatgctctcaggtcgcatttgagagacacagaaatgattttaatggaattaatatattttcagtttttcacaaaaaaaaattgtgtttataacaactgcagattcaatgctgattttatgagtctcacctatagaatttatatttctatattcatagaggcagtataggtgttgacccaacatcgaaagaacacttcgtaaaaaattctttggaacaaggctgaaaatttactcccaaatttagctatccgatgaagataaatcatttaccgtttattaaagaattatcgagattttagtccaaaccaaaagagattatgagcctaagattttgaatttgtattggtaaaagaaattgaacgaaaatttcagaaaaaaatattaataaattgaacgatagagttcacttactacatagtcaactagtgcctagctataatagtttcaaaagacaaaaaaaaacaaaatcggttaactacttccgtgacataattctcattttgatttttgaatccagtctaatttgaaaagtatattcaaaatctttaaatccattaatgataacttttataatacgttgacacacgcaattgtatatacaatattcttgaattttaaatgtaaattctagaatatattgcgatcaccacactaatcaaaatctttgggacaacttgaacccacatttgacttttcttggtcaaatattttggcatcatgcatgatcttctctataaaaaccaaaaggcctcaacgacattcataaactcagtcattatatttatttttgttgtatttcaacgttcaatctctgaaa

SEQ ID NO: 25 - DNA, 450 bp, Arabidopsis thaliana
  misc_feature (1)..(450): Ceres Promoter AtCR2
tttgttttatgaagctaaaatgcttagctcacttggctccatttttccacacgggtgttaatgctcatacgtgacatgcttttgagaaaaagctgaaaacaacttatattaattcattattgatttacatattaaaattttgaaattttagcacaaattacataacatattattctttaaaagttttccaaataaacaatattaaagaatactaattcaaatatttttcgtatataatatcatgactactttctattaacaatttatagtcaaattatatagcgttataaacttataatatgaagacttacgtacttttctaatcaaagagagtatattaatataatattatcaggcactagcattagtcagtggctactgagtaaagctttgggccaccgagagaaaccattttctgagagcacactttcgttgacttctctttaacca

SEQ ID NO: 26 - DNA, 1000 bp, Arabidopsis thaliana
  misc_feature (1)..(1000): Ceres Promoter AtROX6
agctatacatcctccttgcatttattatagtgcctagatttttttttttttttttttgacattcagtgcctagaattttggtttatgtagatagaaaagtgacactcgacgcaaattcgaaagaatgccaccgaaaaaaatggaacgaatgatgtttcgctatgttggatgcttcagatcccatataacaacactactctcaaaattcataaactctcaaaacatattaatttagtagcatcacgaaaatacatgttgttctcaaatcattacaataacttctactcttattagctgaaaagtataaccttatgtttttgtaaaaagattgctaaacgttatatttgtatatcttttcagtttgttaatgtcattagaacgaagtgtaaaatcgcttcaagatttgttattctaaatgtattttagaacaaatattctgaacattaaattattttcaacaaaatttattttgaataacaaataacttgtcatggtcgtaactcttttttttcattctctagattgaatcaaaaataattttgttttgtagtataaaacatttttggtcttttattcaccgttttatagaacattaatatctcaagttctaatatgaaaacaaaaataactgatccctcaaaaaaaaggaagataaaggttgaaagacatgttattctcttcccaaaacaacctaatccaatacctctaaaaatttatctttctcacaatgccttcaaacaaattgtgttctttctatagttaacttgactatattaaaatctctaaagttaacttgacttctgaaacttgtgatctttctatagttaacttgactatattaaaatcttgagcaattaaaccattaaaaaaaaaaaaaacaggagaaaccttgaccaatacaacaaatcctaaaaatggccaacgataagaatcacaacagaaatttaaagactacatgtaactatttatactacagaaaacaccaaatagaatccaatca

SEQ ID NO: 27 - DNA, 530 bp, Catharanthus roseus
  misc_feature (1)..(530): Ceres Promoter CrSS
gatctttattgtttgtattttcataattaattaatccctacaactgatttcaaaaaaaaatctatttgaaaaaaataattctgctactaaaaattttaaaatcttcctataatatcattttacgagtttaatttatttattatcatattaaaacaataatattattcattacagaaattggacaaacaaaaagatctagcaaaagtattatgattttaatatatagtcgaaaatacgaattagacaagcgaaacattttgtaattaaatcgctatttatcatataattattttacaataattagtattaggaattataattattaattgatgtataaaccgataaaaatatatttatgatatatacaaaataaaggaataaaagaatgcataagtatattggataagcctataaatattactccacgtggtacatcactcttagaccgccttctttgaaagtgatttcccttggaccttgtttggtgagtttgtgggcttattatattccttcactgtccctctaagtat

SEQ ID NO: 28 - DNA, 1001 bp, Arabidopsis thaliana
  misc_feature (1)..(1001): Ceres Promoter AtSS1
agcaaccaatatgtacgtccatctttatatttattgaaaaataatgatatattgggaggtcaaaaatcggataagcttatgattctttttgtgcatttgcattttatttacccaacatatggaccataatattatatcttctgtttattatggtctatatgctagaatgtaataatacatcggaattttgaaagactagaaacattgctttgaaatcctgtgtattttgcacccaaaaaaaaataaaaaaaattgtgtgtttatatacgcaatttattcttaaccacatagtagttgtaaattgtaaacgcaaacagtaaacataaaacattccatattattgacgttttacgtttgtgtttacaatttgcaattgatgtttgatgataaacgcatcgatcgtcaatatttgatcaacaagactaagatcattgaatgcgactatgcgaccggttgatttattaacagtatccatttggtcctttcaatatcacaaaaatcttaaatttccttttttttttaatatccaagcaaatattctagggtatttctcaaaagtatatgtattttatttatttgtaatcaactgctccttggaattacacttgtatatattttctgatatctaaatacctacttctactttatcaaaaaaaagtttacctacttctaatcaatcttagacacaagtacataaatttttagaccatgcgtttgcgatcttgcgttgacagtcgtacgtctaccccaaaaatcagcaatgtacgtggattgcaactatttgactttttgttccatgtgcaacttaatctgtcaagaaaccccgagaaacaactctaatctcttcactaataaaataatccgacgagcataacgtgtgccatcaacttaatttacaaaataagaaaccttcttaaaaattaatcaaaaaagaaaaagaaaagttaaataatcaaacctataaatattcgtaacgactacaaagtacaagaaagtctcat

SEQ ID NO: 29 - DNA, 1039 bp, Eschscholzia californica
  misc_feature (1)..(1039): Ceres Promoter EcBBE
atctaacatgtacatagaccctagtatccttccataattttatgattgattgattctatcctctcattaagtctatataaggctaatactggtttcccattctgttggctactatcaaaatgacctaattagcaaagacaatttatctagttgactactatactaacaatggaactccctaaccaaagaaagagaagatatgggtgggtggggggagagtgacattcttctctaggttttcaattcctcccttgatcttaattttcctttctttcttttattgttttagattttcattatcacaagtaatcaacttgagttttcaacaaaaaaaaataatcaacttgagtataaaatttaatataaaatattagattttaattaataatcaactacaaaatttgagcgggacaaataaaaatcacgtgatttttttttataaaaggcaattaaacgttgtctgtaatcatgtaagaaagattctgactgaatgtataaaaaaattaatagttaagacgtggaaagaaccaaaaaccgtagtatatagtaattaattattttataaggtccaaattattattattagtcaatctaaccaggctgtattactattactaaatctattaatttatgaaaaattagctcgtaatctctttcttcccaaatttactcccacgcacacctcgtggtaggtcatcgtgcgtctcttatctattaactcatttgtttttaaatccctaagtcacctttataatcctgattttctaatccaacggtgcgtaattctctaacttgatcttgaagtccgcatctcattcttagaaccactgattgaaatatttgggatttgtttcgggatttgaccggtctcaataatagatttgtggacagacagccgccaatctggacagtatcttacacgtgtgagatgactgtaggatatgtggggtccatagaattagttgaccaaatcaaagttagttgacttaaaattcacagtataaataggattgacaatcataactggcattctcgtagt

SEQ ID NO: 30 - DNA, 850 bp, Eschscholzia californica
  misc_feature (1)..(850): Ceres Promoter EcNMCH3
taacttaatttttcattagttcatggcagctagctagctaggctcctgttttcttatcttcatcaagaggtggtgatatactatgtgtatcttgactgtgatatacaaattaaccatgagacacaagttaggtgggtaggtggacatgagctataggtggctaataaggcaagaaagggagatattttcttcgatcaccaagggcgtaggtggctgggtcatgagctatagttggtttttgataattcttcaatacgtgtggtccatgacattccttaggcaggatgatttggttggaaaactggctataacgctgccggtctttagtctaatgaacgtggcatgaaggatggttagtattgtggttagctcaattcagactcttttcactacatacgattgagaatggttggactttgtttggttttctcactcgctacgtctcagttgatgggtctaaggcctctttcttttcgttttagactaataaagttatctttatcgcgttctcattgtagattatgtaccaatgattatctttttctttctctctcttttgattatgtggtcttttgattatttgtctctctttaagatgagacaaataattgtagccccttttttttctgcataaatgcaatttgtcttgctaccatttttcgaatggaactttaagatgagacaaataattattgagtggtatgagtctatatgatgttgacttcatcatggaccactccatacccccaaaaactaacactatgatctaaagctatattaaaacggtttggttttgtggttagactaccagttcatcctctctatctcctcccaagaatatcaggtagct

SEQ ID NO: 31 - DNA, 610 bp, Papaver somniferum
  misc_feature (1)..(610): Ceres Promoter PsROMT
ttaagtgtttttagttttgttacaagtttggtgaatgatctttgatgatattttttttttgaagtagtgaacgaagtaatgttctacttcccataggatttgctctaacgattaactatgtttgtcccaaagggttgtatgacttacatacacaatattaaaagattcgaggataaggatatgtttattaaccaactcctcaaaacatttagacctggatcaatcagttcgattcttctcagacaattacgacatgtatgaatcgatggatacatttgaaacatatatccttacccgtgccgttacggcatgggttgagacctagtgattacatataaaacaactaattataccaatgacttgagttcgaaactcgtcccatgatcaattttttatcaatcaaaagaaatttatacgaaaggaaactatatgattgccatgactcgtaaatatacacaagcaatatggaggtcctattaactccaatcaaaccacaatagataaatatatcgtgaatcatgtgtggccaatatatacccacacttctatatataagtgcatccacttctctttgttttccaaaacaaaacataaacacaatttattcaga

SEQ ID NO: 32 - DNA, 575 bp, Papaver somniferum
  misc_feature (1)..(575): Ceres Promoter PsHMCOMT2
agcacagaaaaaagcccttgctctttcatgtagcatattattaattaagcttgtcaattttcagtcttatgttttgaagcatattggctgagatttttattatgtgcttgactgggttttccaggaaattaccactggcaggtcaatcaagaatttttctcagaacttaaacttccggcccggcagatgcaagaaaacacatataccacttgcatgaaaccagatatagcatgggctgacctttctcagaattattcccacgagcttcttgcttttctctcgtgggctgatgatatatgtgacactcctcaacagcaaccgccactgtcaacaactttagtactataccaacttataatgatttcttagttaggtcatggtgggggacttattcttttcttttttttttgttgaatattcatatctgcatcctttaatgcacacaaacggtccggcaggcttagctgcatgcacttataaatacaccataaattttgaagagatttcaaaacacccacacataacccaaagccaaagcaaaaaactgtcttctcttcttgacaattatatacagc

SEQ ID NO: 33 - DNA, 373 bp, Papaver somniferum
  misc_feature (1)..(373): Ceres Promoter PsSAT
ttgtcgagagagatgaattacaaaacaaaatagaaagcatcagcatattacattattacatcgatcctttataaaagataatatatacatacatatatatatatatatatataacctttggtcttcaactgctattgaattacaaaacaaaatagaaagcatcagcatattacatcgatcctttattcggaaagaggggtattctgttcggtgagagtttcttcttatccagctcctcgcaaatgaaatgattccataatacctctctaaaagacttggtcattatataagagagggagaccacgagcttcttctaaacaacagaaagtatcatctaccattatcaatcctgttaaacagttaaacactttgg

SEQ ID NO: 34 - DNA, 971 bp, Arabidopsis thaliana
  misc_feature (1)..(971): Ceres Promoter AtWDC
gaaggcttgtactgttccaaccgtccattactttttttgagaaaatttgttgagagggcaattttcaagtttgtaaatttgccacttcaataacttctttgatttgatttgtcaattaggtagttcttacttcttactacattgatgtcatgcactttcatttagctgtcattttagagaagaaattacaaaaacgttattattttaactaactacttatgtggaatactatctcatttcgggggtacattgttttaacggtggcgataaaaagtgttttgtaacagtgaatagagaataatatactgttgacacatgatagtgtaaaagtgaacattatataacgaacaagtgtgtggtaaatactatcaccagggtaaagtaaagacattgttgtattaatgtgacccatgctgacgaaaatatgtatgtctgtgactcataatgacaaaaattattaatcatacatcatgtcatcactttgccttaatttgttggttaatatagagcatatgttggctaataaaaagaagaagaagaaactatcaaaataaaaattacgagaacgttacaaaagtaaaaaaattagatgcatacatgtgttaaatgttttataaagtttaatttgtgtggagtatatcatttttgaacaaataaccataaatagaaaaacaagaaaatactcaattcttaagtcaaaaatagtattaccaaagtcaaaaatagatgtacgcatgtgttcttcgagtatctcaattctttcgaaggtattatatattttatatttatgctaaaattttacagcttctatgatagagagtgacttattatactatataaacgctagaccattgagtgatgcaaatcacaaagcttctttaataacacacatacagattctttcaacagctgccactacctttcgaaatgtaagcatctcctctaatctccatcatttttcagaatgtgtt

SEQ ID NO: 35 - DNA, 500 bp, Arabidopsis thaliana
  misc_feature (1)..(500): Ceres Promoter AtSLS6
gatggcaatggcgttgattctacagagattctccttcgagctttctccttcctatgttcatgcgcctcaaatagtcgtcaccattcacccacagttcggtgcacatcttatcctgcgcaagctgtaattctagaatcccatgtgctcttgtcagataaacagaggaaaataaaccagatcatagtataaaactcaaatctttttattaattgaattcagaactttgtgctcacatatgtcttgacaaaccaaaatatttccaagaaaaagagtgaaacgcatggaccaatatatgtcttcctattttcgtcgcttttcacacgaagtccaaatttgcttcttgcgccttggtggcacgactgtgagggtccaagtaaagcaaaaattaattagtcattacaaaataattgcccaaaatatcaaaatataaagtatttaagacatcaggggtgatgtcatcaccacatccatatcttctccgaaattctaaacatttatac

SEQ ID NO: 36 - DNA, 1006 bp, Arabidopsis thaliana
  misc_feature (1)..(1006): Ceres Promoter AtBBE5
tcctggagctaccattggcatttaataattgtttttgataattagaaagaaaatctttgaattaaatatttaacaatcttcccaaaatctctccatattaactacacgattagttacttaaataaagcttccaaaatatttaatatcatttaattactacaaaattatcatttttgatattgtttttctacatgactataacaattcgattataatcatcaattgtaactttaaaaaacttttatatataaacttttatatgtagtaactttaaaaaataaattgtccccgcggtataccgcgtgttaaaatctagttatatttaaaagttaaaactaacaaaaacgaaagtgtacttgatagaaacatagcttatatggcctaaatgtggtattaagtcttccaaagcaaaccatgaatagtttaagttttctagtgatgtaatgcatataattttatggattttagatagtgttgttttgagaaccatagtgttatttatatagagattaattcttaatttggcccttaatcaaattagaagttattaagtatatccagatatttacttttcattatatttatgtctcatgtcatttacttcatatatcaattgagtacataaacttagattaatcaaacaataaccataacatactgattaataccccaattaagttttagagaagtaactttgaattcatatgactctttttaacttaattttggacaactgaatgtgcattattttgtcgctttacaaacccgtggaaaatgttggtcacgttgctatcatttcacctaatcgtttcgtttgacaaatcaaaccatctttgaatttagagaaaaattcaatggaccttattttatcagaatcgctatatatttattaaaaaaaatggaaactcatttcttcttttacaagaaagaacaatatacaacatttccatcttgaagccttattgtgtgtttagcttctctcttgcatataatattcgctgctagg

SEQ ID NO: 37 - DNA, 1003 bp, Papaver somniferum
  misc_feature (1)..(1003): Ceres Promoter PsBBE
gttcctataaatgtggcttacagaagtcttgactgatgacaccaatgacaatcccgagaagttatctaaatgatctcgactgcaatcaagagcaaattttgaataccacagtaaaattaagtagaagaaatagaaaaaatagagcccgagaaccttactgatttggacaatattgtggttctatctttccgatttgttatttcatagcttttttattatgttattcaacttgatggatcttttttctcatatagtggaaagctaaattacacacaggagcatgaccgactgcttaccagtttgatgtccactcccaaaaaatctcgacttcgaacccaccacaaaatatccatccctaataacggtcgacaataaaggtttaacatcatgtctccttaatccattgccacagtaaaatcttttttacaacatttattttctgaaagaaaacatttacacagtgagcacttcaacaaatgacaccctagagagagtgccaaaaggcataaaactagagatccgcggagggctgtggagtgcgaacaagacatctcgacacttcattatctgtcaaaatatgatgattagctaccacaagatacccaaatcaaggataaaaacgagtaaataaatatcatattcgtttcatgaaaagaaatattatcactattttattttaatttaaaaatatgtaaattggaaaaatgatagccactttttttacggaaacagagggaacataatccgaatctaagtgttagtttgtctatctccatctttgatgaccattgaaatgcaatgtccaatcctaacgaaactggaatggcccgtgacatgtagcacagctgcacagcagccatttgagaaagtcagacgcgtttactcccacgtgcatcgcgtttacatctaataaaatgttagagttgcacgtgcctgcgggttattaaaaccagcactatattttgagcaccatttctttctatttttgacgtacttctccaac

SEQ ID NO: 38 - DNA, 1823 bp, Arabidopsis thaliana
  misc_feature (1)..(1823): Ceres Promoter 21876
gtctcttaaaaaggatgaacaaacacgaaactggtggattatacaaatgtcgccttatacatatatcggttattggccaaaagagctattttaccttatggataatggtgctactatggttggagttggaggtgtagttcaggcttcaccttctggtttaagccctccaatgggtaatggtaaatttccggcaaaaggtcctttgagatcagccatgttttccaatgttgaggtcttatattccaagtatgagaaaggtaaaataaatgcgtttcctatagtggagttgctagatagtagtagatgttatgggctacgaattggtaagagagttcgattttggactagtccactcggatactttttcaattatggtggtcctggaggaatctcttgtggagtttgatatttgcgagtataatctttgaacttgtgtagattgtacccaaaaccgaaaacatatcctatataaatttcattatgagagtaaaattgtttgttttatgtatcatttctcaactgtgattgagttgactattgaaaacatatcttagataagtttcgttatgagagttaatgatgattgatgacatacacactcctttatgatggtgattcaacgttttggagaaaatttatttataatctctcataaattctccgttattagttgaataaaatcttaaatgtctcctttaaccatagcaaaccaacttaaaaatttagattttaaagttaagatggatattgtgattcaacgattaattatcgtaatgcatattgattatgtaaaataaaatctaactaccggaatttattcaataactccattgtgtgactgcatttaaatatatgttttatgtcccattaattaggctgtaatttcgatttatcaatttatatactagtattaatttaattccatagatttatcaaagccaactcatgacggctagggttttccgtcaccttttcgatcatcaagagagtttttttataaaaaaatttatacaattatacaatttcttaaccaaacaacacataattataagctatttaacatttcaaattgaaaaaaaaaatgtatgagaattttgtggatccatttttgtaattctttgttgggtaaattcacaaccaaaaaaatagaaaggcccaaaacgcgtaagggcaaattagtaaaagtagaaccacaaagagaaagcgaaaaccctagacacctcgtagctataagtaccctcgagtcgaccaggattagggtgcgctctcatatttctcacattttcgtagccgcaagactcctttcagattcttacttgcaggttagatattttctctctttagtgtctccgatcttcatcttcttatgattattgtagctgtttagggtttagattcttagttttagctctatattgactgtgattatcgcttattctttgctgttgttatactgcttttgattctctagctttagatccgtttactcgtcgatcaatattgttcctattgagtctgatgtataatcctctgattaattgatagcgtttagttttgatatcgtcttcgcatgttttttatcatgtcgatctgtatctgctctggttatagttgattctgatgtatttggttggtgatgttccttagatttgatatacctgttgtctcgtggtttgatatgatagctcaactggtgatatgtggttttgtttcagtggatctgtgtttgattatattgttgacgttttggttgttgtatggttgatggttgatgtatttttgttgattctgatgtttcgatttttgtttttgttttgacagct

SEQ ID NO: 39 - DNA, 1000 bp, Arabidopsis thaliana
  misc_feature (1)..(1000): Ceres Promoter PT0668
atagagttttactatgcttttggaatctttcttctaatgtgccaactacagagaaatacatgtattaccactaggaatcggaccatatcatagatatcaggattagataactagttctcgtcgctatcacttcgcattaagttctagtaattgttaaagattctaattttttactaaacaaaaactaaatcaacatcaaatatgcaaagtgtgtgttgtccacacaagtgactcaaagtatacgcaggtgggattggaccatattattgcaaatcgtttccgaaccactcatatttctttttttctctcctttttttatccggagaattatggaaccacttcatttcaacttcaaaactaattttttggttcagtgatcaaatacaaaaaaaaaaaaaaagttatagatattaaatagaaaactattccaatcttaaaaatacaaatgaaaccataattttaatttatacaaaactatttaattagctaagggttgtcttaacgtttagaaaataaaaaattatgattgtctgtttaaaattacaatgaatgaataaaaaaaatatgcaatgaatgaaagaataaattttgtacatccgatagaatgagaaaatgaattttgtacaaaccactcaagaattcaaaacaattgtcaaagttttcttctcagccgtgtgtcctcctctcctagccgccacatctcacacactaatgctaaccacgcgatgtaaccgtaagcgctgagtttttgcatttcagatttcacttccaccaaacaaaactcgccacgtcatcaatacgaatcattccgtataaacgtctagattctttacagcctacaatgttctcttctttggtcggccattatttaacgctttgaacctaaatctagcccagccaacgaagaagacgaagcaaatccaaaccaaagttctccattttcgtagcttctttaagctttttcagtatcatagagacacttttttttttttgattagaa

SEQ ID NO: 40 - DNA, 1000 bp, Arabidopsis thaliana
  misc_feature (1)..(1000): Ceres Promoter PT0535
ttagtgaaattatgacattaagtaaggttttcttagttagctaatgtatggctattcaattgttatgttaggctattttagttagtatatgaatttaggcagtctatgcaaatgatttcgttttcattttttcatatgtaaacatcaagatcaagtaacgccattcgagttgatattttttttttaaattagtgtgtgtaaattttggaccgcttatttgagtttgctaatgaagttgcatatatattacgttaaaccataggcaaactaatttgaaacatccgattcgatttcctgtaatttttcttggttaattgaccaaaatcaagatcttcagaaataaaataaaagacgaaagaaagctgtcgcaaagcagattgtgttaaaaaaaagtggattgggctcaaacgcaacttgtccagcccgtgacaattaccctatacgcaagtaagagtaacgtatcactggcaaaagttggtattagttacgatatctttgtcatgggggcatgcatgggcatggcttaagagttaagccttaagaagagtcccacactcgtgactctcatgatcacttgttgtttcttacgggcaaatacatttaactttattcttcatttattcacctatattcttttggataataacttttctctatataaaataacaaacatcgtacgtttcatttatttacaacaagcgatgagaattaaaaggagaccttaattgatgatactcttcttttctctcggttacaacgggattattacagataatgataatctatatggatgctgacgtggaaaaacaaaatttggtgaaacacgtcaattaagcacgacttttccatggctagtggctaagatcgtttcatcacatggctatatcatataatacttggatgaattcaaaataaacgactgagaaaatgtccacgtcacggcgcaccgctttggacttaagtctcctataataaatacaacaccaaacattgcattcca

SEQ ID NO: 41 - DNA, 999 bp, Arabidopsis thaliana
  misc_feature (1)..(999): Ceres Promoter PT0585
tgaagtcatttaatatgagtttgacattaggtaaacctaatctatgagattatagaatgtagcaaaactatcaatgtttcttttccaaaatattttgtggtttttctttttggttcattatgttttgttatttgtgaattattttaatatgaagtaattatattgattttatatgatatacatattattttgatataaaatttaacacttatccattaaaatagcatgggcataatcaaaatcgggactattacgatgaaaaagatagttaaattgtatgataaaataaaatgtgtaagattaaaattttgggttttagaaaattactaaacaaaatatagacaaagtatgttgactattatttaaaatttaaatatcatcaataagatatagttaaagtcattaagtgtatagcaaaatgaaaattctaagattaaaattcgattaaaattttttttactaaattaaatatttaaaaatagggattatcatttactatttacaattctaatatcatgggtaaaaattgataactttttttaaacccgcctatctaggtgggcctaacctagtttactaattactatatgattaacttattaccacttttacttcttcttttttggtcaaattactttattgttttttataaagtcaaattactctttgcattgtaaataatagtagtaactaaaatcttaaaacaaaatattcaacctttcccattattggaatggtaatgtcttcaacaccattgaccaacgttaaggaatgtcttttaatatttttggaacctaaatgctaatactgtataccacaatcacttatgagtattgaagttgagatagaggaggtacaaggagaccttatctgcagaagacaaaaagccatttttagcaaaactaaagaaagaaaaaagattgaaacacaaatatgcgccactcgtagtccacccctatctctttggcaaaagccacttcactctttttccctttttat

SEQ ID NO: 42 - DNA, 1000 bp, Arabidopsis thaliana
  misc_feature (1)..(1000): Ceres Promoter PT0613
ttaatactaacattgtagaaagccacaaaaaagaaattgaaatgtgagtagatgctgagtcagaggtttggtcaatacacaacagctaattgagataatattatacacgtcacgatgacttgttttttctcctcccaacttgttaatttctttattcttaaaattaaaccatcgcaaaaacagaagaacacagctgtttttctcgactcccaatttctattttgctgctaaggacatttcatttcattatttcccaattcaggactccttagattttcctaaatttgttttcctaacttgctctctctcattctaacattttctcatttttttagattatcttgtactttttagtagattattttatcaggttttacaaacatacattgacattctaaaaagggcttctaaaaattcagtgtggaatgctgatatactaaaaaaaggtcatgcaaaattatctacgatttatctaaaattagataatttgccatatataactattaactaataatcgatcctttgattttttgtttagataaaacgaaacagctatatcttttttttttgttatcggattttaatcgaataaaagctgaaaaataacagttatatcttcttcttttttaactaatgaaacagttatatcttaaacaaacaacagaaacagtaaaatattaatgcaaatccgcgtcaagagataaattttaacaaactaataacaattgagataagattagcgcaaaagaaactctaattttagagcgtgtaaacacaaacacgtcttgaaagtaaacgtgaattacacgcttctaaaacgagcgtgagttttggttataacgaagatacggtgaagtgtgacacctttctacgttaatttcagtttgaggacacaactcaagttatgtttgatatctaaggacttgcactgtctccaaatctgcaggaaggactttttgattggatcaatataaataccatctccattctcgtctccttc

SEQ ID NO: 43 - DNA, 351 bp, Arabidopsis thaliana
  misc_feature (1)..(351): Ceres Promoter PT0625
gatcatgatcagtttcaactcgctgtgcccacgtgtcgagagatcggcacgtgcctgagctctcagccgctcataaatacacttgtttagtagcaacagtatactatagtagtcctctcctgtttggcttttagcttgcatcgatggatggatggatggatcgcatgagagggcttcgcgaaggtacggaaccttacacaacgcgtgtcctttctacgtggccatcgtgtaggcgtctcgccatgctacgtgtcccggaggatgtctcgatgccaacccttataaatactgttccattccaatcccatcgccacagccagtgcaaatctgatcgatcaagataatcgagca

SEQ ID NO: 44 - DNA, 1022 bp, Arabidopsis thaliana
  misc_feature (1)..(1022): Ceres Promoter PT0633
cccgatcggccttaatctgagtcctaaaaactgttatacttaacagttaacgcatgatttgatggaggagccatagatgcaattcaatcaaactgaaatttctgcaagaatctcaaacacggagatctcaaagtttgaaagaaaatttatttcttcgactcaaaacaaacttacgaaatttaggtagaacttatatacattatattgtaattttttgtaacaaaatgtttttattattattatagaattttactggttaaattaaaaatgaatagaaaaggtgaattaagaggagagaggaggtaaacattttcttctattttttcatattttcaggataaattattgtaaaagtttacaagatttccatttgactagtgtaaatgaggaatattctctagtaagatcattatttcatctacttcttttatcttctaccagtagaggaataaacaatatttagctcctttgtaaatacaaattaattttcgttcttgacatcattcaattttaattttacgtataaaataaaagatcatacctattagaacgattaaggagaaatacaattcgaatgagaaggatgtgccgtttgttataataaacagccacacgacgtaaacgtaaaatgaccacatgatgggccaatagacatggaccgactactaataatagtaagttacattttaggatggaataaatatcataccgacatcagtttgaaagaaaagggaaaaaaagaaaaaataaataaaagatatactaccgacatgagttccaaaaagcaaaaaaaaagatcaagccgacacagacacgcgtagagagcaaaatgactttgacgtcacaccacgaaaacagacgcttcatacgtgtccctttatctctctcagtctctctataaacttagtgagaccctcctctgttttactcacaaatatgcaaactagaaaacaatcatcaggaataaagggtttgattacttctattggaaagaaaaaaatctttggaaaaggcctgcaggg

SEQ ID NO: 45 - DNA, 1000 bp, Arabidopsis thaliana
  misc_feature (1)..(1000): Ceres Promoter PT0650
catacttaattctaaaaaaacaacacttatagtttataagcagctcttatgataaaaatctttctgagttatagctctgttaaacttgtattcaccccaaaaacggatgtttcatttcttattttttacttggagtattttattgtaatttgtaaaaaaaaatgtaaagtgggggatatcatgaaaaacaacgtcactttgtttggtcacaatatacatttgataaaataatggtcgtcgcgtgatttagttgatttttgttttatcaaccacgtgtttcacttgatgagtagtttatatagttaacatgattcggccacttcagatttgggtttgcccacatatgacataccgacatagaaggttaaatccacgtgggaaatgccaatattcaatgtttggttttcaaaagagaatcatttctttatatgatctcaaaagtatggaattgaaatgactaatgagcacatgcaattggtgctatcttaaaaaccgaacgtctttgaatttaatttgtttttcaccaaaggtacctaatgaaaccctttcattaaaaaataaaggtaacaaacaaaattttgtattggaaaaaacattttttggaatatataatttggtaatagaattatgagcaaaaaagaaaaagaaaagaaagaataatgagcataataaagcctttacagtattactaattgggccgagcagttttgggctcttgatcatgtctagtaatcttaaacagacgataaagttaactgcaatttagttggttcaggtgagctaccaaatccaaaaatacgcagattaggttcaccgtaccggaacaaaccggatttatcaaaatccttaagttatacgaaatcacgcttttccttcgatttctccgctcttctccactcttcttctctgttctatcgcagacatttttgtttatatgcatacataataataatacactcttgtcaggatttttgattctctctttggttttctcggaaaa

SEQ ID NO: 46 - DNA, 998 bp, Arabidopsis thaliana
  misc_feature (1)..(998): Ceres Promoter PT0660
caagtcaagttccaatattctaaggagaaataatagtatactaaacatacattagagaggttaaacttctttttggatttaagtgtgtatgcataggctatttattcttaagtataactattaactgtagctagatttatacaagaaatacataaaactttatgcatgtgaggtagccatgaatatacgtacatgttgcaatcgattatacatgttgtatttggatttctctatacatgttttaacttgtcattctctaagtatatacataccattaatactgtgggcatgagtttatgataagacttttcttttggagaccagttttgttttcctttccacctatatttgtctataggcttcacggtacactagtttacaagtgtttttatatgttctaaataaaattgagattttccggaacggtatgatctgtttgcaaataaggacgtatatataacagtatcaaatatatttgttgttataaggcaataatatattttctgagatattgcgtgttacaaaaaagaaatatttgttaagaaaaaaaaagatggtcgaaaaaggggagtaggtgggggcggtcggcttttgattagtaataaaagaaaccacacgagtgacctaccgattcgactcaacgagtctaccgagctaacacagattcaactcgctcgagcttcgttttatgacaagttggtttttttttttttttttaattttttcatcttcttgggtttggttgggtcactcttcaggtcaggtgtgtaaaaaagaaagaaagaaaagagagattgttgtgttgtaacccctttgactaaaatctaatgaacttttttaacacaacaaaactccttcagatctgaaagggttcttcttctctcttagtctcttcgtccttttattctccgtcgtcgtttcatgatctgactctctggtcttctcttcttcttcttcttcttctattttttcttacttcgtcactgttgtgtctgaac

SEQ ID NO: 47 - DNA, 1000 bp, Arabidopsis thaliana
  misc_feature (1)..(1000): Ceres Promoter PT0665
aaaaaggatgggtaatgggacctattttccccaacatcccacatgcacacttccctctccattctctcacatttatttctttcattctaatttatccattccgtgtgtaacatattcactaataatctcatctcactaactcattcattgattgtgatatgtttatctagaattagtgttttaacactgtgtctacatatgatttccttttcattgtatgtgaacatgttaactcactaatcattttgtattttcgagttaacatgagtctccacttcggtagactaaagtaaagataggtttgagtataataaagtttaaaatttgctttaaaatcaatatttataaataagtttttatcataagtgatttttgtatgttatattggaccttgtataaacagactacagaagaaaattatttatgagaacttgtaatgttagagtggacctcgtataaactaattatgtgggcttttaccataaactatttatgaaaattattatggcccacaccactataactaaagcccacatatttagcagcccagtttcattgtaagagacatgttcgctctggaactagaattttctggtttttgggtatttgttttcttatgtgtagagaaatgatggtaacgattaaatgttgtgtattacaatttacaatggtaagacgattaatatatttacacacaattttgttgttgctgtaacacgttagtgtgtgtgatgatagaatttcataaagctttaactacgaggggcaaaatgttaattctaaatagttgacagcagaaaaagatatgtatacataatataaggattaaaacgtaaataataataaataaggcgagttaaattaaaaccctgttaaaaccctagcttgaaacacatgtataaaaacacttgcgagcgcagcttcatcgccatcgccattctctctctcatcaaaagcttttctccttgattttcgcattctttagagtcttaacgcaaag

SEQ ID NO: 48 - DNA, 999 bp, Arabidopsis thaliana
  misc_feature (1)..(999): Ceres Promoter PT0672
cagccgtaaatcctccataaatttattttgcaagttttgctcattatataatgagcggaatttatgatataatcgtttgtaataatgttatgttttgatcaaaatttgaaattaaaagtaggtgagaacttgttatacagtgtagataaggtggatcttgaatataaaaataaaatttataagatgtatttaaagcagaaaagcataaaactttagataaaataatgtaaaaatgtgttagcatcaatgttgggatattggccgacccgaacttaatcaatgtcggaagccattacttctctcccaaaagacctttttccttcggagaactaggaacttcctcactacctttcgcttaacgtgaaagccataaatttcatatattcataaaaatcagaaaatctaaaactgtttagtatcacctgtttttggtatagactattggttttgtgttacttcctaaactatatgatttcgtacttcattggatcttatagagatgaatattcgtaaaaagataagttatctggtgaaacgttacttcagtcatgttgggtctagatttacatactactatgaaacattttaagataataattatcctagccaactatatgttctatattatgggccaagaagatatagaactaaaagttcagaatttaacgatataaattactagtatattctaatacttgaatgattactgttttagttgtttagaataaatagtagcgtgttggttaagataccatctatccacatctatatttgtgtgggttacataaaatgtacataatattatatacatatatatgtatatttttgataaagccatatattactccttgacctctgcccccatttccttttactataaataggaatactcatgatcctctaattcagcaatcaacaccaacgaacacaaccttttccaaagccaataataaaagaacaaaagcttttagtttcatcaaagacgaagctgccttagaa

SEQ ID NO: 49 - DNA, 1000 bp, Arabidopsis thaliana
  misc_feature (1)..(1000): Ceres Promoter PT0676
aagatagtacagtttcagtgttttgagaaaaaaagctgaactaaaactaaaatgtttaaggacacaatatttagtttcaattagataattcaacagtttgaacaattttttttttttttttttgaagtcatttatttatacaatgttttaaaacgcattaagcatttaggcagccgacaaacgcctattgtctaactgtaaataggcgcttccacttaggttcatattgcatatttactatatgtgtatagtgacaaaaaccaatatttctcttattttggatgaaggtatagtagttgttaaatgttcaatataattaagcattaatgacaaataaaataaaattaatttagttgataaaaagataatcttataaaaagatcgatgaatagatataatggtttactgaattctatagctcttaccttgcacgactatgtcccaaggagaggaagtaccttaactataattctgaacataattttgtctatcttggtgagtattatatgacctaaaccctttaataagaaaaagtataatactggcgtaacgtaataaattaacacaatcataagttgttgacaagcaaaaaaacatacataatttgtttaatgagatatattagttatagttcttatgtcaaagtacaattatgcctaccaaaattaattaatgatttcaacaggaagtctgagatgatgggccgacgtgtagttacgtttcttgaattgtgagagatggtatttattatactgaagaaaacattatttactaaataaattttcatttcacatcttctgtaatcaatgcgggtagatgaagaagttgttaatacgatggccaaccatatggatctcttttttggcgtttctatatatagtaacctcgactccaaaggcattacgtgactcaataaaatcaagtcttttgtttccttttatccaaaaaaaaaaaaaagtcttgtgtttctcttaggttggttgagaatcatttcatttca

SEQ ID NO: 50 - DNA, 998 bp, Arabidopsis thaliana
  misc_feature (1)..(998): Ceres Promoter PT0678
aattaaatgaaaccgcccctaaattaggagggatttgggtaagtggtaacacattcactggaaacatgtgaagaaaggaggatgtcaagtagctgaaaactcagtatagtaaccaacggcttctcaccaacctttcattaataatttggtcatccctatatttttattcaacattttgtttttcaatagcttagagcaccttaatacctttcagtgtttttttataaaaaaaacaaaaattgggattaatcatcaatccccaaatgtaacgtttacttagattatgttcatttttctatacacacaaatcatattcttttgttttaatcttcgaaaaacgagaggacattaaatacccctaaaaaaggaggggacattactaccaacgtacattaacatgtttgatagcaaacgatttattttgttcgttttgaaaaggggaaagtaatgtgtaaattatgtaaagattaataaacttttatggtatagtaacattttcgaataataagagagggaaaacactcgccattgtcggcaatttagaaccaatattagaagggtttttttagagaaaaaggacttaaaagtttagagaccttaacaacaacttatttagaaatagacatgcttaagttgacaacagcgagtttattttctatatcgaagaaaaatacgaactttttcttaattagatttcgaatgcatgcactatcgagaatcgaccgtcacaagaaaaaactaatatacatactgtacatatctatattcaatattggtggggatgggtttaatgtgtatttataattcatggataaattcacacaataaggtccatgaaactagaaggtaccaaaaataagcattaatgactctttgccacttatatatatgattctctcatagtaccattttattctcccaaacctatcttcttcttcctctcttgtctctctcgctctctctcttctacattgtttcttgaggtcaatctattaaaa

SEQ ID NO: 51 - DNA, 1000 bp, Arabidopsis thaliana
  misc_feature (1)..(1000): Ceres Promoter PT0683
gattgaatgatgagtgtgcacccttgtattactaataaaaaatttagcaacagttataagctaacgtcatccatgagtcattcattagattcactatttgcgttctcaaaaatcgaattgttaaaatttgagaagctctaatatacgagtcaatgagatgtggcaaaagcatgtccttgaccataaaatttcgaggggtcaactcattagataaggacaagaatcaaccaattgaaggcgtcttctataacaagtttctttattactaatattaaagtccaatggggtgagggggagaagaacttaaataaaaggaaataattggtaagtgaataaaatctaaatacgatactagatgattgatttgtgctagtgcatggtattagatcagatatgtgttactattcgaattcaaattggcatattccatgttgttgataagaaaattgtagaagtgtaaaagctgagttactatattcaaactagtggtttacataaagtgagacaacaactgtttcacaaaaatgactataaaatagtaagtagtattaggtcaattgattttaaaattttaatcaaattcaaatttgtgatataatcaaatttgtttatagaaaatgttaagaaatcaattttggcagaactaattcagtgagaaacaatcatttacaaaaacaattttaacattatttaacagtaagatttgacatttaacccgttcgtgtgaacccatcatatctaacatggctctacccatgacgcctccatgccatggacaattttgacagatcagaagttctgaacgtggacgaggtaagaacaccatgatgatacgattggagttagttatgtcgccaccgacatcactgccaatctcattaataaaagtggtactaaatctctaatctctattaactataaatataacaaagaaccaaaagaaagtttcttatctctcttatctttcataatttccaagaaacacaaaccttttctacta

SEQ ID NO: 52 - DNA, 1000 bp, Arabidopsis thaliana
  misc_feature (1)..(1000): Ceres Promoter PT0688
acgttcagaggcatcgcttttgtacaaattgaagcgggtttgttcaatatttaaaataacacaggaaacattcaaatgtattattgatgttgcttaggtttgtgaaatgatatgaaccatatcgtatatattactagatttttcttatatgttttaagggtagtggggctgacctatcattctgtttggcattaccaatcagactatcagagtattcaccattcaggattccataactagaaaaagaaggggtttacattttctcatactgtataattttctactatcagagattttatcgattacattaatctcatagtgattattctgatttataaaaaagttgacaaaataattaaaaccagtattttataacaagattgtctctctcccatggccattattttgacctctgacttatttaaatcttaattaacagcataatactgtattaagcgtatttaaatgaaacaaaataaaagaaaaaaagaacaaaacgaaagagtggaccacatgcgtgtcaagaaaggccggtcgttaccgttaaggtgtgtcgaactgtgattgggccacgttaacggcgtatccaaaagaaagaaagggcacgtgtatagatctaggaaaaaagaaagaatggacggtttagattgtatctaggtaccaggaaatggaacgtcacaccaaacggtacgtgtcggatcctgcccgttgatgctgacggtcagcaacttccccttattcatgcccccctgcccgttaattacgtgtaacccttccatgcgaaaatcaaacccttttttttttttgcgttcttcttcaacttttctttttaaatcaaaccttttctttttaaaatcacattgcatttcctaacgctcaacaaaatctctctctactaatatctctctctctctctctctattgttgaagaagactcataatcggagattgtttgtttttggtttgctctgtaaattggagaagttttgttagagatcaa

SEQ ID NO: 53 - DNA, 1000 bp, Arabidopsis thaliana
  misc_feature (1)..(1000): Ceres Promoter PT0695
aacattttctttaacttactcttaaattttaatagtaagttgatgcatgttatgttgatccgtcttgatcacaaatattgttttatggacgaattctttgacagtaaatggctatagtgactcagcttggagcatcccgatatgaaaacaaagtgcagtattgtgtcgtggtcatcactaacgcactttcctagaactatcgcgcgtgtttgacctatgcaacacaccagatgtcatgaacgtatacttaaatagaaacaatgatatagacaattggctatattctgtcatggaacgcaaaccggataacatgtctattagattcatcggacttgatcatggttatgtcttaatagacgaattctttgttaacgattggttaaaacggctcacgttagagcatcctactatgacttcaaaattgataaatattacatggaaatcactttaattttagttagaaggtagttaatttagatattcttatttaataaattaaaaaatagaagaaaaaaagatgagaagagtttttgtttataaaataagaaatatcttttattgtaattttaaaattaaacaaatttaatttatattaaaattatctttgttttattgttaaggcaataattatttttttggtgggaattgttaaaacaataattagtatactgttaagtggtcctttaataataagataacgtgatttaaaaaagaacgagacaggctaatatagtagagaggaaaaaatacaatttaggcccaataaagcccaatatagagttgtgctcaaacacaggtcttcgccagatttcctatgacgccgtgtgtcaatcatgacgccaagtgtcattcaagaccgtcacgtggcgttgtttctacacataggcgatccatacaaatcagtaacaaacacgaaaagagcattcatatgtacgaaagtagaaaagaagagactctttgtgataaaactaagtaagaaatagcataaaagtaaaaggga

SEQ ID NO: 54 - DNA, 1000 bp, Arabidopsis thaliana
  misc_feature (1)..(1000): Ceres Promoter PT0708
gtttccaaaactagtattctttatttgctctattcattatatttttatatttgtaacgtcccgaccgtctttattaggtttcgacaatcacttctcggaaggtcgtccatcctgaaattactctatcctaaacatgtttaactataaaattctctcgaaacttttgtaacgtatataaccacataaattctcttaaacttatttgcatacaccattatatttctgaaatcgatatgttacaatattatttaatatttagattacttttactgaatcgaattaaatatcaaatcgaaacaaatctaatctaccaaaaataattttgttataaacatttcttgcctagttctacctcatatacattttagttaaagaaagaaatcacaacaattcccataattcaataattaaatccacaaaatcttggagtaagtaagagaaataaaaagatagtatcttaacataaacaattcaaagatgctctctcacacaattcacacacacttacaaaacaaaagacagaaacaatgttttcattcaaatcaaaagaagttataacactagtacaaaaaaagctcaaattctaatagtaactctttttatttcccaattacccaaagattctctctcacttcacaaaactagctttgagagtcgtgttccacaaaatccattaaagctgaaacggttttgctcaccattcaaacaaatacaaaattgcaaaaccccaaattataacaaaataatataaaaattaaaccgctaaaaagagtgaaccaacaaaaatcgccgaatgtgtgtgtaatgagaaaaccgacccatcatcccaatcatctcttcccgtgtcactctcttcctctcccacgtttcttctctcttccctttatgggttttaacttctccttcttcttcttcttcaatcttcagttttcaaattcaacaacaattcacattttgatttcttcatcatctctctctctctcgcttctctctcaaatcg

SEQ ID NO: 55 - DNA, 1000 bp, Arabidopsis thaliana
  misc_feature (1)..(1000): Ceres Promoter PT0710
tagtgcgcgtggggagagggaatggtgaaaccttagtggttaagttatgaggaaaatgataaaaggataaaacaatcaaatgcagcttgaaacggccataacataaagtaccttatggtggtgcgaatatttttgtgtttctttcactcttttattgctgaaagctacgacacttgtcttaatatattgtttccgcaagtcacatgatctactttttatttaacgtctagaaacgccgagatatatgatgattagtatatcacgtctatgcaaattgttagttcgtgtttggccaaaagatatcgagacatgtctgaagaaccgagtctggttttgagatatttcttcaagcattactatacaatagaaaaaggagacacgcgaatatgataatagcaaaaggcataaaaaggcgaaaattaaagaaaaacgtaaagtgatttggcctcaatcaacgggaacgtatcttaattttagaggttcttcttttacttttgagacgagagagtttgcgtctttgcgagctgctttggttgactaaacattatcatattgaaaaccaaaatacaacggaggaatatttgtcacagtttcactttcacattgtttccttaacgtttaatcaaccttgttcaaaatttctatagttgtaatcatcattgtttacaaaattttcgttcaaagatgattttaaataaaattgtgaaagaaaaccttttctgaaataaggattggatgatagtgttaaaagaaaaatatgaactgaggcaaaaagaggagtggtccccggaagattgtgaaatgtgtcatctaaaccagccagacgtagtcacgtgttctctctagctttatgaacttccttagccagcaccatcattgtgattgtagtatatatgtaaccctaccttcatctctcccattttccattctccatatagactcctttacaatatacaaaacctatccaaaagcgaagaagccaagcaaacatattataaaa

SEQ ID NO: 56 - DNA, 1002 bp, Arabidopsis thaliana
  misc_feature (1)..(1002): Ceres Promoter PT0723
gtcatatcttatcaacacgtcaacgatcaaaacctttagcctattaaattcaacggcttagatcaaaacgaaactaggtgggtcccacttttaatatcgtggctgcataacatttcctcgataactgaagccgttgtggtctttctcagaatctggtgcttaaacactctggtgagttctagtacttctgctatgatcgatctcattaccatttcttaaatttctctccctaaatattccgagttcttgatttttgataacttcaggttttctctttttgataaatctggtctttccatttttttttttttgtggttaatttagtttcctatgttcttcgattgtattatgcatgatctgtgtttggattctgttagattatgttattggtgaatatgtatgtgtttttgcatgtctggttttggtcttaaaaatgttcaaatctgatgatttgattgaagcttttttagtgttggtttgattcttctcaaaactactgttaatttactatcatgttttccaactttgattcatgatgacacttttgttctgctttgttataaaattttggttggtttgattttgtaattatagtgtaattttgttaggaatgaacatgttttaatactctgttttrcgatttgtcacacattcgaattattaatcgataatttaactgaaaattcatggttctagatcttgttgtcatcagattatttgtttcgataattcatcaaatatgtagtccttttgctgatttgcgactgtttcattttttctcaaaattgttttttgttaagtttatctaacagttatcgttgtcaaaagtctctttcattttgcaaaatcttctttttttttttgtttgtaactttgttttttaagctacacatttagtctgtaaaatagcatcgaggaacagttgtcttagtagacttgcatgttcttgtaacttctatttgtttcagtttgttgatgactgctttgattttgtaggtcaaa

SEQ ID NO: 57 - DNA, 1001 bp, Arabidopsis thaliana
  misc_feature (1)..(1001): Ceres Promoter PT0740
tgtggccactaaagatttacccttaaccgggcccatataagcccacgtcaagtggcgcttatacgctctccgtaagagagccaacatttggtatgtaatgttgcaaattattcttcaagacaataaattcaaatataattcaatattgtccaaatatagtgatgtacttcagttgtgcacatagaaactccactaaaccaacttttagatagatgcattcacaaattttcaacaatgtcgcgaaagtctaatccatcaccagattctaacattttaattattatatttaactatacatactctaatcagcatgagtcaaacgtgtacaatagcccaagcatataataagaccaaagtcaaactcaaataaatgtctccaaactcaaaacttgaaaaagacctaattattacatggtagatatgactttgtcgacaagtaaaccaactaatcctcgaagctaccttctcttcccagttattatgtgtgatcgatttataaatctcttcttctaataacacctatatttttcttatgatgtgaataaatataaaacttttaactttaaaacatatttatccgaaatattgcacttagatttcaaatagataaataatagtactatctaactgatattgaaaagacctaacacggaaaacagttttataaaaaatcccaaatgtgggtaattatcttgatttcttgggggaaacagaaaatggattaagattaatcggagtcgtgtcaagcagctcgttaataactgtagcaagttgactgagtaagcatcaacgtgtcatctccgtaaagcccattatttctagtctcgccgcgtcttctcttccacgtagcacttcactttttctctccttttgtttcctttggaacacaaacgtttctatttataggaataattacgtcgtccgtatctgtgtcggaacatagatccaaattaaaagcgacttacttaattacatatcgttcgtgtttttttcttcaaaaa

SEQ ID NO: 58 - DNA, 1024 bp, Arabidopsis thaliana
  misc_feature (1)..(1024): Ceres Promoter PT0743
tcgattggcccgatcggccccaaaatcaagctgagccgcttcaaacttcagcttttgaaatcacccccaaactcatgtcctcttatcattataactaaaggatctttcattttatttaactcatcgtcttgcactacccaacccaaaggttccaactatacccgaagctttctaaaggtccaaagactttttttttcgagccagactattcaagccaagaaaagccaaaccccacaagccagtacttttcaattccatattataaacttatctgtcttgttttagtcccactaaaaacaacagaatttaatttaggttgagctaaaacccttgacaaaagtgtatagtcgtcgattcagtagcacactcatcactcatcagatttgatagttgacctaaagtatgactactccatttcaactaacaaatgaaaataaaagagacctaagggttagaggattgaaactatactctcaagtcttttatcactaggctactaccagctagttaacttgatggatttaagcaagaaaacgtagaatttatattcgagcagattgtttagctaaaaaagcttgggtttgaaattgccttttctcccatataagcacgtcggttcctaaataactctttctagcggagagtgtctttccaataatttaataaaaatggtgtttgtatatcaaaaaaaaaagaaaaaagaaactgatcgagatagaacgtttgcagttttataaacaatttaaaaaacaaaaaaaattaaactcaatgtattttttattaattcacaaacaataataaatcataggatcgaatatttacacggtatcaaaacctactcgccgctactatataaaaattgaagtcaaatatcaaccgcaattattaaaccagcaagacaataattcataaacttaatataaacataaataaattaatgttacacaacgatatatggtgagggttattactatcttcttcctctcaaaacacatctcctaaccttaagctttagacggcctgc

SEQ ID NO: 59 - DNA, 1000 bp, Arabidopsis thaliana
  misc_feature (1)..(1000): Ceres Promoter PT0758
agctagccacatcagtgaccaaaaaagataattaacaaaccaaataaaataacaaattttgatcatttggaataaaatttataaaaggaacgaaagcgccttctcacgggtcccatccattgaaatatattctctctttttgctctatataataataacgcgtactaatttgtagtatatattattacaaagtcgatatttgattgttttgtgaacgttgatatattaattttcttggatgatgacaaaaaaagtcatagaaagtaacgtgtgaacatagcattaacaaaatacaaacataatatataaccaaatatatgaaaataggataaaatctcattgaatagatcttcttctattcaaatatataaatatttgtttgtctataaaattaacagagcattcacattatctaaaataatagtaaaatcaaaataaaactaaataaaaataactctggttttataacgattgattttaaatattagtttttgttgtaaagagatcattatatatgtctgtaatatttttatactgagttacatgatatttagttattatagcgtaattaactaagataagaaattaactaaagtgatattctgattattattatttttgttaggacacgtacgtggaaaaactaaacactataggttacaaaacggtataataaactcaccattactggaaaatgtttgcatttgactcaataagtaacttattataagttactgatataatgcatagttttgaaattcttaaataaattattttggtttcgcatgaaaatatgaaaggagagaaatttattattgtcacttatatatatatacatcgtaatcattttttcgtgaataattctctctcccattccattatttctcagtatctctctttctttcccttactttattgttgcttttaaaccttcaatttgctcataaaccaaatatataatatcaaaacaaacaaacaaaaaatcagaattcccctaata

SEQ ID NO: 60 - DNA, 921 bp, Arabidopsis thaliana
  misc_feature (1)..(921): Ceres Promoter PT0829
aaagttttgaattattgggaatcaatttcgaagttttgtaattctttgggggctaataggatattttattttcttggtttcgtctattgttgtttttctatttatggttgggcttttagaactctggacaggcccatgtcatatgttttcccttctccttatatttttcatttttcattttgttaaattaatgcataatatccaaaaacaatttaaatttttgaaggaaccctttagttacggctccgaagctttcacaagtgagaatgtgagatcaaagaaggcaaatggaggattttaaaagttaaaatcatcttttatctgcaaaagttgacaatttttttgtatcaaatctaaatcatcaaactctcttaaactacaagagcataacaacctctatgtaatccatgaaataatctgcttgaaggacataacataaatcattatggctagagtgactaacttcaatcaaatcctcttaactctagctcccttacaatggtatcgtaaaacattatgcattagggattgttgtcctaggaaaataaaataaaaatccccacagaccaactaccattttaacttaaaaataagcttcgtccgcgacgaattgttttccatcctaaaaatagaatggtgtaatctgctaatggtttagttccattaacttgcaagttctattgaaagcctaaatgtcaataaagatattaaaattcggagtcaaaagacaaatgaatcaaaagcaacaagacaagtcagctccattcttcactacccatcttttacaataaatcatctctcttttcacaaatttcaaactactctcattgccctttagctttgttatagagccaacactacagagagactcacacacttgtttcaataattaaatctgaatttggctcttcttataaacta

SEQ ID NO: 61 - DNA, 763 bp, Arabidopsis thaliana
  misc_feature (1)..(763): Ceres Promoter PT0837
aactacaagggagacataatatcaccatctggttcctgttatcatctgaagatttcttgttttaccttccagtgataaaatgatccttataatacatatagatatattaaattgctgtattttaagattatagatatataaggtacatgagagtgtttatttaaaaaaattcacttggaattcatgttttgtgatacgttagattggaatccatttgggaaaagaagaatcatctgttcttatgtctcaaattttgacttcattcacttttcttcttgtcttttaagaaagcttccacaatctaactgttcgatgtgaaaactgagattcgagtaagaaaatgtgaactgtgttatactgttttttaattagataatttagattgcactcagataaattaataacattcctcgaatacttttatgtgattggatatattaggtatatctgccaaccaaccaataaactgctatgtttaaacaaattaaataaattagtatatgtttactcaagaataaagaagatagaaaagaaaattctatatgagctaaatttgctggaggaggcatcggacgtgggtaccagacctttccaagcacacgagtagtgcttagccatgtcatgctaacatacaccatttggttcatacaaaatccaaatcaaaatctatttttaaaatcttttgcacacgtctttgaaaaacacctctcatactatagctacggaagcttcaatttcaaggtttgtctaaaagctaacgatt

SEQ ID NO: 62 - DNA, 751 bp, Arabidopsis thaliana
  misc_feature (1)..(751): Ceres Promoter PT0838
atactggtatgcttaaggttgaagccaagatctctgtcttacccaagtaaccactttctattagaagggatcaacactaagaatatggagatttaagcctaagggctaaggcggttctcaacaatacatgatgtgaatacaatcacagacgatttactgaggtttgttgataagatcttgatcagtctctgcatcatctgttcaacaatctcaatctttgactgtttgctttcggagccataaacagaggaatcccttattccctgttataggagcaatacaccaagtattatttccatggctgaaattctcttatggaaacctaattgttccattgaagctgtaaaatcgaatctggtgaatattctcgagcaaagccgcatgctaattatgtcaattcagaagagtttgattaggagactcgaagcgagtttgatgatctttcttgatgttcaactccgattgtaagggtataattgacttttcatgtattacggctccaccacctgacactaaggcactctttgtccatctcgttggtatcatcggattcggatggtaaaaataaaaagagcagaggaaacttgttactcatgcaagcttctcaggtgccacgtcactccattacgtgtcatcttcacacaccatctcgctcaaaaccgatctcatttttcaaaccttaaaggcagaagcaactgattaagttaacactcttgagaagctctcgattaagcttgaacttggaggatca

SEQ ID NO: 63 - DNA, 669 bp, Arabidopsis thaliana
  misc_feature (1)..(669): Ceres Promoter PT0848
tctctttaaatcagttaactaaccgtttatatatttacgataaggtttgaagagattattgataaaataatacatttcataatcccgcgttcaaccgtttaaagtaacatttaagttgactatatctaattttttttccattaaatatggagctggtaaactttatcaacttctaaaaagtgtaacaacaaaaattaggtcaatcacaattctgttttttttattattttggattgacttccaattgcaaatagtcttagtgatcaccattatcatacatatatacatcaagtaggtttcatcatgatataccacaaagtatttgacaagccatatggttttggatcaaaaagtcggtccaaaattaatgttttatgtgcaagaaccgacccattgtacacacgtgttaacatcttcaagactttcatctctatttttcttttggtcattaagatacccattgatccgaatctgttacattcccacctacttttttaatttttactatccactccaaattaaacacaaccgatgattttaataattggaagcttttaaaaatatttcaaaacaagcctctttgtgtttgtctatatatatacacgtaataagaaggtgaatgaatctcacagcttacttgttctaaggcttccaataacgaaaacagta

SEQ ID NO: 64 - DNA, 702 bp, Arabidopsis thaliana
  misc_feature (1)..(702): Ceres Promoter PT0863
cgggaaacgacaatctgatctctagtccagtcgattggcccgatcggccgattataaacttacatgagacaagtataaataattattataaacttattaagtttaagatcaaggcttttgtgcaatgtatcaatgaatgttagatgtgatatgatgaaagcaatgttttaaacacatacatagtcattgatcggaatgtgtgttattagaaatgcatgcctaagccgatagggttatctatgtttggtcttggacattatagccaaatttcgaatctaattcttccaatatatatttttttttttttgcttagggccactactagtattgcttatcaattttaagagctcatgaaaatgcaacaatatagtagttgcaaatccttgtttcaagagaaatcaaagggccacttgtgaattgaataataataatatttgcaaataacctttcactaaaccataccaacaaaaccacacagatttggcaaagacataacctttgggagacgtgaaaaggctcaaaatttgacaattgtccttacaaattcgctcattagtgcaattgtgagatttgtttgcatccaaatccaattcataactcacactcgtctcaaattcgaaaaggcctgcagggccagtgcactgggatccaacaatgtcctccgactcgtccaagatcaagaggaagcggaaccgcaccgcg

SEQ ID NO: 65 - DNA, 435 bp, Arabidopsis thaliana
  misc_feature (1)..(435): Ceres Promoter PT0879
ttctaggaagactggtcaagctaagctgtttctgttttttgtttttgtactttactttttgtttgctagtgggaactgggtttattgggccttgaagttgataaaagatgaataaaagacatatcgcctaaagcccatatgagaagcagaagacaaaaacctccaactttgggcataaattttgattatagttaaaagtccagacccaatttggcacctggcttagttacgattctaaggcatgacacctgcctaatatgtttattacagaaaataaagagaatcagctaggtgtcccttattgaacacattaacaaactccaacgacactacgtgtcttcgtgactcttactatatccaaaaacctatagctaaagctgaattttccatgattagtatagtcccaaccaaaaaaatactgaagaaggcataagc

SEQ ID NO: 66 - DNA, 397 bp, Arabidopsis thaliana
  misc_feature (1)..(397): Ceres Promoter PT0886
agtgtatttgaaaacgacattgaagaattaatatatttttttttaattttagttttttatagtacaaatattaaaacaaacaatcctaccatatcataacatttgtaaataacattttaagttttgttttgagttttaattaattttctatgacaaaaaaatgaagtcaatagactaagtgaatcatatagtataaataaacacaatttaaatagtttcaaataaatttagaaagaataaaacaaatagaaatcagaaggtgtctgtttcctcctcgcaacatacgatcaaagagaaacaacttgaccctttacattgctcaagagctcatctcttccctctacaaaaatggccgcacgtctccaaccttctcccaactccttcttccgccatcatc

SEQ ID NO: 67 - DNA, 1024 bp, Arabidopsis thaliana
  misc_feature (1)..(1024): Ceres Promoter YP0007
agcagaacaactatatttattgtgtcacataaatctgagatcatttataaccaccaaagaacctatacacagtaaatgacaaatgtatctccctctatctctattgcccatatgtagatgctaaagtaagatttctcttttttttaatgtacttttttttgtataaagtatattccataagaaaaaggaaaagcttgtttatggatcaattgaccccaaaaaaagtttttagatcaaagcccaatataaaaaaaaaacacagtagtgacacaaaggaacttaaataaaccatgaattgatctataaacagtagagatcgataaggcgaacattttccatgtgaagtgtcttctttcatctataatatttttgacatccaataatttcctctataatatcattcacataattgatagaaacattatgttagaattgtccacatcatttgagctgtaatatattctgttttaacaaattatatggtagttgcttaatcttatgtccatcttcttctatgcatcgttttcgcgcctagttgtccagtccatttcaactacctacctctaattcttatcttaaaacaacattttttaatttaagtattatgctcaaagactaactagatagaaaaccgttattaaacattaaacgaattaaaagtcttacatggaaaatgtaggtttataaaccacgagttatgattgacaataaaaaaaatgcaaatcatcaatcaaaagagacttgagtgcgactctatatcaaccattgcaattaaaattatctatcacaaaaattttagacagattaagttaatttagtctaaattcactaatttattttctataattagtaattaactatatttatttatttacacattttctgataatttagaaatttgcatgaataacaaatataagattttggaaattagtagcaaatttaattaataattatttttgcctaaatgaaccaaactataaaacctccacatacaccagtcatcaaatttacagagacaaca

SEQ ID NO: 68 - DNA, 1000 bp, Arabidopsis thaliana
  misc_feature (1)..(1000): Ceres Promoter YP0008
ctcgagagatgaagtcttagtaatgtttgaacaaacaataatcacgttttccatcaaattcgagcatttaaagtttatattactacatgccccaagatgataccgtccatctcatccgaaaatatttctgaaattgcgctaagacaacaatgtttgctcaaattcgatcatttaaagtttacaaatctctcatcaatcttacaaacttctcacactaaacagaggtacatattttcttataaagacaaaaggttcgaacagctggcttctcaactcgagttgtttgtcagggcctctcttcactaactacaagttggtacttcaaatattggtggctagcttcacgtgatattgtctacaaattaaacccatgaaaaagctgcattaattgttccaagtgaaccctgaggagtgtcaatagtctttgctttagtgtgatcattaaaccaaatctctaaattcctaatttgtactaacatttggaacgtatttcctactcttctccctgctccaactcccaaaaataagattagttagatttctataactaatatacatgtatactcccaaaaacagtaaaaccatattaataaagctaattttgcatagatttatttcggtaaaccggcggttcaagttggggaaaaaaaagacaaacggtctaaagtcatccaaagacaaaaaaccaaagacaagttgagagagacgagaccaatcacaacattgcttcgtagattgcgtgacatcatccttgacggctactttcatttgtgtcttatttggataaaacgcacgtgtttaattcacgaaccttcatagcaataagaaatttccattactttcatattttcaactttttttattacccattacatgcttaaaatattaattcacaagtctttgtcaaaattcaatattttccaggttcatgaaccctttttatctcaatctactctataatatctccctataaattacaacaaaacctctttatttttca

SEQ ID NO: 69 - DNA, 999 bp, Arabidopsis thaliana
  misc_feature (1)..(999): Ceres Promoter YP0019
gatataagtagaatcattttttgccgccgtttctcgctaacacaccgaaaactgaatcaaatctcctagctcttctacgcaaaatcgagtgcatcgacaatggcggaacgtggtgtcgaacgtggtggagatcgcggcgatttcggacgtggattcggtggtcgcggcggtggaagaggtggtccgagaggtcgtggtcgccgtgcaggtcgtgctccagaggaggagaaatgggtgccagtgactaagcttggtcgtctcgtaaaggaaggtaagatcacaaagattgagcagatctacctccattctctcccagtcaaggagtaccagatcatagatttactcgtcggtccttcattgaaagacgaagtgatgaaaatcatgccggttcaaaaacaaaccagagccggtcagagaacgagattcaaggccttcatcgtcgtcggagatagtaacggtcacgtcggattaggagtcaaatgctccaaggaagttgcgacggcgatcagaggcgcgatcattctcgcgaaattgtctgtggttccgatacgaagaggttattggggtaacaagattggaaaaccacatacggttccgtgtaaggtaaccgggaaatgtggatctgttactgtacgtatggttccagctccgagaggttctggtattgtggcggctagagttcctaagaaggttcttcaattcgctggaattgatgatgtctttacttcttctagaggatccaccaaaactcttggaaacttcgtcaaggtatgtactttcacaatggctgttttggtttgatgaactctgaattaggcagtgaaaaagtaatcattaccagttaagtgaatttatattgaagattaggatttagctgattgtattggtttgagcatgtgagtttgtgtttaagattgcttgaattgaaatgctttaggttgtttgattacgctaaattctgactaatgtaattcaaattgttgttgtttttttttggtc

SEQ ID NO: 70 - DNA, 1024 bp, Arabidopsis thaliana
  misc_feature (1)..(1024): Ceres Promoter YP0028
gtcagtgaagtcgattggtagtacttgaaacacttggttggtttcatgtatttggcctatatataaacaaacatcgtaattatatacggatttttttcggaattttacgccatatctgtaagtatatataacatgcatgtcgttttcaaattcatatgatgaacgatccacgtaagtgctactactcctacaatattgcatgagagagatatgtatttataaattttattttgaagaagaaataagagggaaggttacttgggtggatcgatgtgaaaacaaaagaagaaaaagcgaaacccactaagccattacatgatatcgaccttcttatctttttcctctttattttatttttctcatcttctttttgtcaggacttttttctacttaatgaaacctccaaactatctaactaatacactcccatgtagaataaagaaaattatataagatattgttgatattttgtaactagaaaatatatttgctctgtaatttttcgtaagttaaatcaacattttaaagtagaaacaaatattactgcaaaaagtaggatcattatttttgtccaaaatctcagttagctatagggttgtagtaaaaacaaaacacattcttgatttgccccaaaaaataaagagagagaagaatattgttcaaaagtggtctcttctctctctaattatgttttcactaaacccaattagattcaaacagtctacaaagtccaaaagataaacatgggacaacaattcgatgcaaaaaatcctcttttcatgctctttttttattctctagtcttttaaattactaataaaaactcacaaatccaccaaacccattctctacaactcaccttcatctagatttacccactcccaccgagaaacacaagaaaaaaaatatacatatataaatatacaagacaacacatgatgctgatgcaatatacacaacaaagtattaaatcttagatattgtgggtctccctttcttctattcattttcttattcattaaaa

SEQ ID NO: 71 - DNA, 1024 bp, Arabidopsis thaliana
  misc_feature (1)..(1024): Ceres Promoter YP0039
ccgttcgagtatttgaaaatttcgggtacacccgcctaaataggcggaccttatctagtatatatatacatttgaactatattgtttactttttagttgatttaggctatgtcatgacattgacataaatctacctgttatttatcacgtgtaattcgtgtaaagtgtaaactagaaagttcaaatacgtatttgtttttgttctgttatataggattgtcatagttgtaaatctacaatttattacaacatgaataagtacacaagcaatgtaattggatttaattgctaaactctttacatggtcaatctaaatttgataagaaatacgtcacatattactaagactgatagtttttttgttgtcaccaattatttttgttaaattgacgaaaacaattccaaaaactcaaatgtacaaaatcatacagtctcacaaacatctcatagagaaagatataaatctcccatatgggaacgataacacgaggtcgaaatactattcgtaaaactaaaacgccttagttataaatcgttagttgtaaccgcggtcgagaatacatacagatccacgaaactactactacacatgctgctgaattggaatttggaaaagaccatcttctttaggaagagctcacccaatgagtgacaaaggtgtcggtggcttgttttctacccatatgtatacatcaaatggtagtttcattaacgtttggttttgagaaaagtaagactttggctagtagctaggttcgtatataataaactcttttgagaaagttcatcactggtggaaaatgttaaaccggttttttctcattttttccgccatgttaaccaccggtttaaaaagaccgtaacacattgaaagattaataagggtatatttgtaattacggtttgctggcaatttttaattattattttaattagagaaaatagagaagccctatcaatgtacatggtatatatataaaaggcaaaaccctagaaaacgatactattcgactcagccgtcctt

SEQ ID NO: 72 - DNA, 1024 bp, Arabidopsis thaliana
  misc_feature (1)..(1024): Ceres Promoter YP0050
aatctgatctctagtccagtcgattggtacttgagggaaacatcatatttttaaaccttgtctcagtaagctaacacacaccccttgtgattacttatccatgtttatccacaagaatgcagttggattgagatattttcttctttgttgaaatcaggcctcaaggtgttcatgtggtctgcaaaaaaattcccaaaaataaagatagtgacatctgaaatcgataatggattagacgaagagtttcgtgttattccttggtatgggcgggtttggggacagatattttggcacagacgaggactaggccactgtggtcctgcagcattaggtgtcccttccatgtcctgcattacattttattgatggattcatcaccctatctactacaacggctacacaaactatgaagagttttgtttactaataaatgcccaagtgaggggtcgatcgaacccgggacacgtttttcagtttaccatatagaattatccttggaacccttgatactccatagaacatcaccacctctgttgtcatctcaggaatccaggttcaaacctagtctctctctccctagtgggaggtatatggccactgggccaatgatgacaaaatgcaaaaaaaataaaatacatttgggttcattatctaaaatatctcttgtgtttgtaagttttggttgcacactcgtgtggttgaagtgtgtgtgagaggtactatacaatacactctgcttttgttttgtacctatctctttctcttctccacatatccaagactttggggataaagctgagatcattggttgccatttggttgtgtagaagcaatcacccatttgctttatccgaggttgataaatttcctcgggttctccttctgacacgtatgacaaattctaatagtatattcctcgtagatattacctatatattctcaatagttgcaggtacttaaggctttgtcttggcatcctcgtcctcttcagcaaaactcgtctctcttgcactccaaaaagcaa

SEQ ID NO: 73 - DNA, 999 bp, Arabidopsis thaliana
  misc_feature (1)..(999): Ceres Promoter YP0086
cttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctc

SEQ ID NO: 74 - DNA, 1024 bp, Arabidopsis thaliana
  misc_feature (1)..(1024): Ceres Promoter YP0088
tcgattgggattactacttcatctagtaaggttctgaaaacgtttgttgttgataaggaagattcgtctcaggttattactgttgatcttcaaggtttgtgattgtgacgcttatacatgtgctgaaactgtggtgtttatttattgaaaacaaaaaaaaagtctctcttgtagtttcattgtactaaatagaaaacaagaaacgtttttttctttaatcttctacattgataatattggatcaaaggattgtttctgcaagacacaacacaaacatacttatactagtttacttctactaagtactaactacatacccatacacacacttgcacctagactttacttctagacatcattaccctaaggtagaaccaagcttacaagcaagttttaccgacaactcttacattacaactctagtctgtagtctttaacgtagacttactaactagtcattagtggtttaattttttaaattttcatccatatgtttttgttgtagatataaactaaagtcggtcacatttaataattgtcattatgtccgcgtaaaagtcaattcagctattggacatttatgaaatgtaagattttctctctcatttccccgtgcgtgaagacatgcattggtttttctgtaataatcaacaaatccaaaccccttttcgatctttatttggacattgttagagacaaaatttctctatagtctttttcctaatttgataccatgtttttgtttctgcacaaatttactcactggtttaactaactatccacttatttatgattttaccattaggcgtcagctagccctagtcaaatttgtaaacaagccaagctatctacataaatcgagatgtcattaacgttaatcgtcgttaattcgaatttgaaaacatagatagctttagcagtacaatgggcaatggtaagaagaatagcaaaaggcccaatatttggtttgcagaaattaaagccttaaaaaaaagcccacagatatttgtcaaagaaccctaat

SEQ ID NO: 75 - DNA, 1024 bp, Arabidopsis thaliana
  misc_feature (1)..(1024): Ceres Promoter YP0092
aaagattgagttgagagagatggtggagacgcagaacagacaaagggagtttaccatatagtgctctaaagggcaatgagattgcagtgatgtggctatccggggaatcatcgcaggttattccttcccatgagcaacaatcaatggatgggttccaattcagaggagaaacagaagaagaaacgtttccagagaaccacagtagggattctcgatcttgcgagttgcagagagcctctgaaactgcaatagaaaggacactgatgaaaagaacacactgaaggagtatgccaatcatgtgaaaactcagagcttgtattggtcttgtggttgatgaagttctcacaaaacctttggctttgaatctcccctcattagtcatggtgagaacaagaacaagacgagaaacagacaaagaagatgaaaaaacttgttggccagtgttgactaagggggaatagccccagacataacaaaattagacttgtcgtacatctttaatatttttttatctgtttctttgtcctgacgctttcattattcctgtgatcaattttctcataccattggtccatcgttaatcctttcttaatttcattttctacgtaacatgagaggagaccaagtcctatgagaacagttgacgtaacagtggttgttaagttaagttaaaaagaggaagctagtgagagtgaccgttaggtagagaagtgagatctttaaccactcttctttctctctctctctgcttttttcgtcgtctttcacatctactgttcgcaaactctcttatgcttccaataatggtgataccaattgagacttgcaggagaatctcctcttctccacactctatcaactggtcagccatggaatggtcgtttcagtttcaatattcctggattctttttaaggattcctgtttctcttctgttcctggtatattcttaacgacgaaattagtatcggatcctggtaatacattttgaagcttttaagtaccattgcactgggatccaacaat

SEQ ID NO: 76 - DNA, 1020 bp, Arabidopsis thaliana
  misc_feature (1)..(1020): Ceres Promoter YP0096
gaggtcagtgagtcgattggtgcaaaattgaaaaattgaagggtgaaacaaatttaaagataatatctattaaatcctctaattttaaaaatttagcaaaaattgtattttcttatggatctgttagttcacacgtatcttaattagtaccaaatcatatctaatgattagtgataaaactagttagatatctatatgtgtctttaccatttaacttgaatccttcttcttttttttacgtaaacaacttgaatccttcgttaatacataaatttaaagcattttttctttaattctattgatcggtatatatttactataagttttagctcatatgcaatttcaaatgatatgcttttaaattttgtctaggtgtgatagttgtatctttaacataaatcttatagcaaaattatacttgatattctaaatttatctatttgctcttgtgaacctcatattagtctagagaaactttgaaatcctttcaattagttgtatgtccaatacatttttactaacatttattagtctttttaattaagattattgttagaaaaaaaaagattttttaaaaataaataatatgttttagatacaatgtgagttaggcttcttatattttaaaaaataaatttatttcatacttaaaaatagtttggaatttcaatttatttggctgaataccataaaatatgtcaatttgaaccttatacccattgactatttggtgttagaaaccctttaacaaaaaaaaactatttggtgttagatatcaaaataaaaaaagtttaaccattggtttcttatattgaattggatattgttacatgtattaaagtttttttggtttaattttgaaacgttgatagaaactattaagtttaagtttggtagtatatttatttgtggaaaatttaattgccattaaatataacgtcaactttttttggttttttttgagaagttacgttgtgattttgatttcctatataaaagttagattacgtcattttttaa

SEQ ID NO: 77 - DNA, 1000 bp, Arabidopsis thaliana
  misc_feature (1)..(1000): Ceres Promoter YP0097
ttcatctttatatttaagagtttaaaaactgcaacttttgtttttctttcactaagtcttatggccacagttaattaaaagcagatgaaaggtggtccaatggaaaaggagaatgtgattgggctagttgggagagttctgatgtctagtgttgggtacacgtgtccgtcagttacacatagcattaaatcagacggcatgtcattattcaaatctagttcacatagtacgactaatagctgataaattaatgattatacagcatatgaattatgaattcaaaaaaaaaaaaaaattgaaaatgttaaggagatgctatattttacaaaattcatcgcaatgctttctactaatttgctaagtggtcttctccagttagtcttgtcgattccaagcgatattattaaatcttgaagcatcgctcaaagcattatagcttaagataaccaaattgttattaaaaacacctagtgaaatttttaaattaaaacaattttgatatctttgtaatatctaatactactctttctgtgtctaaaaggattaattttcaaaaatttcacacatattaaaaaaaaaaaaaaattactagctaaacaattttcaataatcataaaacaatagtaacttaataatttttttttattttcaaaatagtccttcaagtttacaattcattttagtattataatcaacaaaatttgtattaaaaagttggaaaattaatctttgtggaacaaaaaaatctagaaatcattttttagaattagagagaggtttgataaaaaaaaataaaaaaaaatagagagaggtagtacatactaaacgatgtgatactactattgacaaaatcttaattctcagtttagtagaataaactagaaggaatgaatgaagtaaatgcgaatccaactactaacaaaccctacttagtcatcatattttcccatatgaaatccctatataaacccatcatcatctcccacttttttcatatcca

SEQ ID NO: 78 - DNA, 1004 bp, Arabidopsis thaliana
  misc_feature (1)..(1004): Ceres Promoter YP0101
ttctcgttctctagaatattgctggaccggattaggtcaatattattgggccagattagatattgaattgtcgacgttgcttacgttacgttatatcttgtttaagaattaaacctatcgacttagtcttaattaagaaaacattgccttaaattctctggtctgcgaccgtttttttgaccgttaacccctaattaaagaaacaaaataattatagaaagagcactgaaatgtgattattttaacagtactcttatgagaaaattcgtactttttagttttttttttgtacaaatctctaagaaaaacactactactaattaagaaacgtttcaaacaattttattttcgttggctcataatctttctttctcggtccgggactaaccgttggcaaaaaaaaaaaaaaagttgacaataattattaaagcgtaaatcatacctctcaaataaaaacttgaatttggaaacaaagacaactaaaaaactcgaatttaagagaattcctaaaatcaagtgaagtatcatcacttggtaaaatttcataaccgttggcttctatttctatgtgtgccttggtttgcaggagataatatttcatttccaaccaatgatattcgtacacatagtcaaacaaatgtttgtctttgttattatattgagaaagaaacaagaaagagagagagagatagataagacgaaggaagtgaagcttccaagcgcccaccgttaaaaatctcgtgtgcaagtttcaaatacaagtggccggtggtctccataatttgatcgtcatccaattaaaaaggaagaaaaagcgtgttttatacaagaaaactcattaaaatagcaagtctagaaatatctcaacactaatctaccacgtctattacacacacacacacacacacttgatcttaatttattttcaagattcaagaaaatacccattccattaccacaacttgaccacacgcctatatataaaacataaaagccctttcccc

SEQ ID NO: 79 - DNA, 1000 bp, Arabidopsis thaliana
  misc_feature (1)..(1000): Ceres Promoter YP0102
atttggttgataacgttttcactcgactaattatatacttcagaaggatagtaatagaataccaaaataattaaatgattggttagtgccttagtggagactttttaaccgattctaatagactaatgatgtagctaagcatttatttgggatcatcactgtttgaaaacgtgaaatgtgataaaagttatgaaacgattaaaatataaaataaccgtacaaaacattatgtaccgtttttttctctgttcttttggcgatttggtttagttcgttacactctaaatgttattgcagatatatatataatgatgcatttgcatctgaggaacatataattccggttaacacttccaaatcttatatccgtctaggtagggattttataaatcatttgtgtcatcatgcgttatgcttgtcggctttgaccataacgcagagatatagaactagcttttacttaacttttagatttattatttgatctagagttaagtggagatatatagtgtttttgttagattattggtggatgtgagagtttgtctttagtttcaagttgagaatataaggcaagaggagactctgaggcaatcagaggttttgattggcaaaatatccaaaaggcccaaaccaagtcgaagcccatctcgtacaaaaaaagaaagagatctgtaagaaaaaatattctttgatattcttacaaaaataagtgtaaaacttttattagtcaaaatcttcaatctttaaaaactctcatcactcctacgaaagcgcgtgagagttatgagacattccttaatagcattactcacaagtcacaagttcaaaacgtctgactgaaacagaaacaagcctttgttgaagtcttgaagaagagacattagtactcgtcgtatagccataaaaggtaatatacgaaatttcttcgctaatctcttcaccttcctctacgcgtttcactttcactttataaatccaaatctcccttcgaaaacat

SEQ ID NO: 80 - DNA, 1004 bp, Arabidopsis thaliana
  misc_feature (1)..(1004): Ceres Promoter YP0103
gttttgaagaacaatctggatcgaaatctaacataaggtcatcgtattcaagttacgcagtcaaggacttgacatcatcctactctggtctgaggttaccacttccaaagatgggatttttcgactcggtatgcttcctaagaaattcgttttattgaacctagcaaatatcttgtaatgtaagattcctgagatgatgaagaaaaaacaaacttttgttacagcaggagaacggagagaaagaaaacagagaaccaaatgctcttgaagcaaacagaagaagaagacacaaatccaaacttgagacttcttctacaccagaaaaccgcagcattctgggacaacgcaaaacacgaaagtgaaacgggcaatgatatatatgtcttgggtgcgttacaaggcatcgtttgcaactgttgagttggataagtcaactgtcttcttttcctttggttgtagtagctgccttttttttcctttgttgctttaagaaatagcccgaaaaaaagaatgttctacatttcggagcagaaaactaaccgaatgagtttttggtcggatcatcggatcgatcagatatattttgagttacgaactgttataaaaaaagccataattttgtgttgagtttgcaaaataccttataacttgttatttgagattgcacctccatatatattaattcgtaagagtatttattaagtaagctttagtataaatccttttttcctttaaagtaagttaatgttctactaaataatagtaaagttgaagaaccgctccgtttttacaccatgcacgtgttatctaacaaagaaaatatggtacacctaatggctaatgcaaaggacaacacaatgaaactaacttgactctgtgttatagaaacccatagacatctgcatacatcctagtatttgtataaattggactcaaattcctgaggacaatcatagcaaacaatcacatcatcgcaatatacataaacaaaagaggaagaaaaa

SEQ ID NO: 81 - DNA, 1003 bp, Arabidopsis thaliana
  misc_feature (1)..(1003): Ceres Promoter YP0107
taacaatccttgggaacattgcatccatagatatccggttaagatcgatctttgaactcataaaaactagtagattggttggttggtttccatgtaccagaaggcttaccctattagttgaaagttgaaactttgttccctactcaattcctagttgtgtaaatgtatgtatatgtaatgtgtataaaacgtagtacttaaatgactaggagtggttcttgagaccgatgagagatgggagcagaactaaagatgatgacataattaagaacgaatttgaaaggctcttaggtttgaatcctattcgagaatgtttttgtcaaagatagtggcgattttgaaccaaagaaaacatttaaaaaatcagtatccggttacgttcatgcaaatagaaagtggtctaggatctgattgtaattttagacttaaagagtctcttaagattcaatcctggctgtgtacaaaactacaaataatctattttagactatttgggccttaactaaacttccactccattatttactgaggttagagaatagacttgcgaataaacacattccccgagaaatactcatgatcccataattagtcggagggtatgccaatcagatctaagaacacacattccctcaaattttaatgcacatgtaatcatagtttagcacaattcaaaaataatgtagtattaaagacagaaatttgtagacttttttttggcgttaaaagaagactaagtttatacgtacattttattttaagtggaaaaccgaaattttccatcgaaatatatgaatttagtatatatatttctgcaatgtactattttgctattttggcaactttcagtggactactactttattacaatgtgtatggatgcatgagtttgagtatacacatgtctaaatgcatgctttgtaaaacgtaacggaccacaaaagaggatccatacaaatacatctcatagcttcctccattattttccgacacaaacagagca

SEQ ID NO: 82 - DNA, 1024 bp, Arabidopsis thaliana
  misc_feature (1)..(1024): Ceres Promoter YP0110
gggatgcggttccgcttcctcttgatcttggacgagtcggaggacattgttggatcccagtgcaatggtaatataaaacaagaaaacaagagattttataggacaatcactaaatgacatttaattgattaaacatttattcattaataattgtatgttactaacttcaacatttaataattttgtttaagatacgtttacatcagagactattaatatttttacaggttgtaactttaaactttgtcttgaatcgaacatgactatagattttgggcaaacttaaagataacaacatttccgttttttttcaaattattacaaatcaaactgatatattagacacaacacgattacacgtaatgaaaaaagaaaaagataaaaagataaaagaagggatcgattctgtttggtctggtttagtgagattcaaagttaagctcttcctttcaagacatgccttcttaaaccgggaatgtgaacgtttgtaatgtagtccgtccagttaatgcttccaacatcaaatccaaattctctcttctcgtcctctgacatattctccattaatctctggggtattgctgttatcaaatctgtaaaagaaaccaaaaaaaaaagatgaaaactttgcgggtaccggttttgtctgctctaagaattagaatgttaatgagttctgtcttaccttccaccatagaaagtgtatggctcataaatagtagcaaggtgtttggcttgttcaacagatttcttgcatataaactttagcttctgcatcatcttactatccactgaactcataccactcatcaacccactccgttcttgagcatctctccacaaatgatccgagaaatcatcaacggaattgaaaagtttcatcaaacgcaccataataggatcacctttagagtccatgcatggagatgttttgtagtggttataaagaagctccgctaagtcttcgaaaaccagcgggtttatcgccgaagaagcgatctgatacacgtttatttcaggttcc

SEQ ID NO: 83 - DNA, 1024 bp, Arabidopsis thaliana
  misc_feature (1)..(1024): Ceres Promoter YP0111
cgattggatttagtctatacattatagggcgcaagtttgtggatttaagaattatataaaaacttgaaatatatagtttttatgcattctcctcttgtgtaatacataaaccaaatatgagataggttaatctgtatttcagataatattaaattccaaacaatatttttacttgttataagaaggcaattaatatctctctgttaatggcaagtggtaccaagtagtattaaactattaatgcaatggaagagtactgttggaaattataatcctctatcacacattcaaacagatctcctgaaatcttctcttccaaacttgtacttctctgatccaaatgtaggctccaaaatatagacatttaccatttactaagtccacaactcctttcttgtctccttcaaaaatgactcttgtgtaaccaccatatgactccgacagttcggcattgccatgatgagagcttaaaaattcaccttcctgagcatttcaagtcttcactcccttagcttgacctgaaccaagataaaatgcctttgtcgtcccgtaatatccatcctgctttggacggcatcatagttacattcgatccatcctatttacaatgttattttagtattaaaaacatgacaataaatttgttgttaaacatattcaaatacaatatgattggatttataagtaattgtaatatgaaatgtccttagtaatatgttaaaaaatacatagatacacacacgtactaaaagaggcaacgcgggagatgtcattagaggaagaactaggaagcagagcgttcatgcaaaatgctaccaaaaacgttaatgcaatatctcaactaatcagcacagtccatttcatactgagaatgtaaaaaccaatcagcatcgtccattttttcatctaattatttgttaactcttaattggccacaacttccaaccacatgacgctctttctattccctttatatattcccatctcaaatgttcttggagacacaaaatatcataaacatata

SEQ ID NO: 84 - DNA, 996 bp, Arabidopsis thaliana
  misc_feature (1)..(996): Ceres Promoter YP0115
gtcgattggatgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttac

SEQ ID NO: 85 - DNA, 1024 bp, Arabidopsis thaliana
  misc_feature (1)..(1024): Ceres Promoter YP0117
gtcagtgagtcgattggatcacagtcctttatgataaaacaaactcataattattccaccgacaacatgcgttttaaattattttttcttaaattatattatattatattgatatcaacctagctaaaataattcggatggcgaaatcggacaatttttaatagaaaaaatgggtatgaagatagtctatgattccgttcttagcgactagagggacctgctcaaatctcccgggtgatacgcgatgtcaagctcaatagaaccccacaaccgacgagaccgagaaatccttgatttgggctagaagattttgaaataaatttaatatattctaagtaacttgcttaaattttttttcaaactctaaagacataactaacataaagtaaaaaaaaaaaagttaatacatgggaagaaaaaaattaaactaatgattagctctctaacgtgtttaatctcgtatcaagtttttttttaaaaattatattgctattaaaacattgtactattgtttctattttgtttagctattattcttgtgaaatgaaaagttgtgtttattcaattactaaatggcaatatttatcttggaaaactatacctctaattggattaggccctagacatcctctttagcttattgacgttaaaattattcccaaaactattaaagtttagtagtttgaaagatgcatcaagacctactcagataggtaaaagtagaaaactacagttagtgtgattatattttaaaatatataaaacaatcttattaaactaaatattcaagatatatactcaaatggaagataaaaacatttagtctgttaccactaccagcctagctagtcactaatagtcactttggaactgagtagatatttgcatcttgagttaccatggactcaaaagtccaaaaagagaccccgagtgaaaatgctaccaacttaataacaaagaagcatttacagcggtcaaaaagtatctataaatgtttacacaacagtagtcataagcaccattg

SEQ ID NO: 86 - DNA, 1000 bp, Arabidopsis thaliana
  misc_feature (1)..(1000): Ceres Promoter YP0119
taccaaaaataaggagtttccaaaagatggttctgatgagaaacagagcccatccctctccttttccccttcccatgaaagaaatcggatggtcctccttcaatgtcctccacctactcttctcttctttctttttttctttcttattattaaccatttaattaatttccccttcaatttcagtttctagttctgtaaaaagaaaatacacatctcacttatagatatccatatctatttatatgcatgtatagagaataaaaaagtgtgagtttctaggtatgttgagtatgtgctgtttggacaattgttagatgatctgtccatttttttcttttttcttctgtgtataaatatatttgagcacaaagaaaaactaataaccttctgttttcagcaagtagggtcttataaccttcaaagaaatattccttcaattgaaaacccataaaccaaaatagatattacaaaaggaaagagagatattttcaagaacaacataattagaaaagcagaagcagcagttaagtggtactgagataaatgatatagtttctcttcaagaacagtttctcattacccaccttctcctttttgctgatctatcgtaatcttgagaactcaggtaaggttgtgaatattatgcaccattcattaaccctaaaaataagagatttaaaataaatgtttcttctttctctgattcttgtgtaaccaattcatgggtttgatatgtttcttggttattgcttatcaacaaagagatttgatcattataaagtagattaataactcttaaacacacaaagtttctttattttttagttacatccctaattctagaccagaacatggatttgatctatttcttggttatgtattcttgatcaggaaaagggatttgatcatcaagattagccttctctctctctctctagatatctttcttgaatttagaaatctttatttaattatttggtgatgtcatatataggatcaa

SEQ ID NO: 87 - DNA, 999 bp, Arabidopsis thaliana
  misc_feature (1)..(999): Ceres Promoter YP0120
tagtttttgatttaatctacgtttttcttaatcataaatgggtaattattagtttttgcaaaatcaaaatccaaaaattgttctaaacactgcaaccatttaaggcctatatcactcagaaaatttctggtgggagaactaatcgtttgtcctttctaaatctcacatattagaatttagaattagtgtgctacataagaatattagttcagctcggaacaactattttttggtaaaacagagaacttaaacaaatgcattattttatcaacatgcattttgaattgaatataaaatttcataattgtaaagacataaattacataaaattttacatgaaaaaatagatatagaaagaaaatgaaactaactgatgatatgctctctaaattttttaatctcataacaagaattcaaattaattagttcatatttttggttaatataacatttacctgtctaagttggaactttcatttttttctgttttgtttagtcagtattcttaatgtgaaacggaaagttgaatttattcaaacttaaattcaatagcattaattaaaggcgaaagctattatctctacatgtggttcaaactagacatccaatttaattagcttattgacgttgaaatgttttccaaaactactatagtttggcaatttgaaagatgcatcagaactactcagacaggtaaaagtagaacctctagctgtgtgaattgtatgttagtccataaagaacatcttgtaaacttcatacttaagatatatattacaatatatacttgaatggtagataaaaacgattagtctgattgctagcatactcacaactatttggaaatgagtaagatattggcattctagagttactactatggagacaaaagtcgaataaaagagacctcacgtgaaaatgttacgagctagtaaaaaaagcatttacactaacggtaaaaaaagtatctataaatgtttacacaaggtagtagtcatt

SEQ ID NO: 88 - DNA, 999 bp, Arabidopsis thaliana
  misc_feature (1)..(999): Ceres Promoter YP0121
ttggattttttttttgttgagtcagcagaccatctaatctctctttttccaccacagcctgctttctatgaagcatttgggcttacggttgtggaatcaatgacttgtgcactcccaacgtttgctacctgtcatggtggacccgcagagattatcgaaaacggagtttctgggttccacattgacccatatcatccagaccaggttgcagctaccttggtcagcttctttgagacctgtaacaccaatccaaatcattgggttaaaatctctgaaggagggctcaagcgaatctatgaaaggttggcccattctccttgacaggcttaacaatacaacttgtatcgcttcaacaagatgatggcttaataaggatttttgcatgtataggtacacatggaagaagtactcagagagactgcttaccctggctggagtctatgcattctggaaacatgtgtctaagctcgaaaggagagaaacacgacgttacctagagatgttttactcattgaaatttcgtgatttggttagtgtaacccactgttattcttttgatgtctacatctactttacttacattattcttttcttcggtttgcaggccaattcaatcccgctggcaacagatgagaactgatcatgacagggtaggattttatttcctgcactttctttagatcttttgtttgtgttatcttgaataaaaattgttgggttttgtttccttcagtggtttgattttggacttatttgtgttaatgttgttttggctgttctcttaatatcaataacaaataaatttactggttggtatctaagatctaacaatagttactatttttagaggtaaagacaccaaccttgttatattggtcagagagctaaaaccttgacttgttgggaaaacaaaactctaatgacagaaaatctgacatgatgccttataattcacagcctcatgttctacataaatcctaacaatagcactttgtttct

SEQ ID NO: 89 - DNA, 1004 bp, Arabidopsis thaliana
  misc_feature (1)..(1004): Ceres Promoter YP0128
gataaactgataatggaaaagaacaaagaaaccagtttttaactatttgcatatgtaatttatttgttgcaaattatatttagttaaaatgtttcctctatttatatatatatatatcagtcaagcactatgtataagaaatgtcaatttataaatttttacatgtcctttaacagaaagaaaatgaatttttacatgtcattcatagagagtcactcgtttatttcttatatagagaataacacactcacatgcatatgcatgcaatatgatacattttatgacaaagataatcaacggaaacggtcaagacataatttgataaacaacttgcacgatgcacagatctgatcaaatatataactctttaacatatccaaaatattcaaaaagaaaaactcgatccaaactagcaacatcacgctcacgcggtaggctaaaaatttattaatctccaaaagtctttcttatgaacactgcaaacacaacaacttgaaaagtcatataggtttagatgatgacgcgtattggctatcgcttaccggagtggctcataaatacaataaacaatacgtaaaagtcaaagtcaaatatatttagtcaactataaccattaatcgggcaaaacctttagctgtcaaaacaacgtgaaaacgatatttgtatatatcatcaagaatcagtagataagagaatgatttaatcccctgactattacaattttggtgtaataaacagtctctattggtttttattctttgttttaatttctcatgacctatagagagaattaggtagtttcgaaaattggctaatcaacttttgaaaactactgtctactttgcttaaattctctacacttagtttcggataagataattgtcggactaatagttaatcccttgacaatctttgatattataaaaggtttagttaatctcttctctatataaatattcatacaccagctttcaaaaatatataatccaaacaccaaaaacaaa

SEQ ID NO: 90 - DNA, 1001 bp, Arabidopsis thaliana
  misc_feature (1)..(1001): Ceres Promoter YP0137
gtggcacatgctgaaaccccgagcatctctccggaagacacgcgtcgttcgctccaaagaaaacagtcacagctgccggagaatctccgccgtcttcttctgccaccggaaaaactctctccaccactttcagtgcccacctcgtgttatatccactgtatcctcgtagcaccatatcagcctaataaaattttatgtatcaaattttaagacatagccgaaactacactatactagacaataataatatgatttgtttcctgaaaaattatggtttcatgagaaacattaatcatctataaaacaaattagctatggcatcgaagagttatcaatcaaaactgatgaatctttacttaatatatacaacatatctttaccttgcggcggagaagatcggcgagagaagcaccccagccaccgtcactaaaggattcttcagtgatggaatcaccaaagagaaaaaccttccgtctcatcatcttccacacaatcttcttgagaaaatctgagagataagaaaggtgtagtggttttgctgaagtgatcgtgtttgatttagtaaagaaatgctttatttattgttgggggaaacataaataaataaagtaaaagtggatgcactaaatgctttcacccactaatcaccgacctttcatggtttattgtgaaatacactcatagatagacatacaataccttatgtacgtaaataacattttatttgtcgacacttatgtaagtaacgcatagattattttctatgtgattgccactctcagactctcagtttcaaccaataataacaataactacaacaacattaatcataaacatatgctctggtttacaattaaagcttagattaagaaactgtaacaacgttacagaaaaaaaatgttatttacgttttgtaagattagtctctagaatcatcaccgttttttatatattaatgattctttcttatatataaaacctttctcgaaatacccatgaaa

SEQ ID NO: 91 - DNA, 1001 bp, Arabidopsis thaliana
  misc_feature (1)..(1001): Ceres Promoter YP0143
atacaacagatggcagatatcgagttaaatacgtgaatcagccgttacgatattttaaaactagaaaattatttaaaaatattgcaaaataccatttaatttcattgttcataaaaaaaagaaattcaaaaacttaaaaactgattcaaaaatttggattaattctcattaacagtcttcaacactacaacaacatgtttctaatttattttatattttaataattaaacaatatatacgtctgcacattgttgctccgacataatctagtataaaaatagttgcagcatatgtgaaaagcaagcagcatttatcactcaatacttttaattttatctgttgtatgtattaaggttttgtagctttaagaaaacgcttataatataaaataacttctaaaagatatttcatgcgtatacaataaatatttgtgaaaaaacatttcgaaaacgtgtacaatatataaactattgtgttatcttttgacattcaaacaaatgttgacaatgtaattttatccatgatatgattggccaattagctgcgaggtaaaaatccgtatacgagtaaaagtaagataaaatttcgcaagaagatttttagcaggaaatctaagacaagtgtcatgaacgtgtcaatcaacaaacgaaaaggagaattatagaatccagattcgacgtaccacattaataaatatcaaaacattttatgttattttatttttgctctggcagttacactctttttcattgctccaataaaaaaatcactcgcatgcatgcatatatatacaccatagtaaactccgcctcttcttcattttaaaagtatcagtttacactgacacaatccttaactattttcctttgttcttcttcatctttattacacatttttttcaaggtaacaaataatctttttaagtcacttttatactctttaaatcttagattgatatatgaatgcatgttaatatttcaagatttataggtctaccaaac

SEQ ID NO: 92 - DNA, 1003 bp, Arabidopsis thaliana
  misc_feature (1)..(1003): Ceres Promoter YP0144
aaacgttgcaagattattgattgtgagaaagagtgctcaaggtagtactgatttctgtaaagctcacggtggtgggaaacgatgttcttggggagatgggaaatgtgagaaaatttgctagaggaaagaagcggtttatgcgctgcgcataacactattatgtctcgggagaacaaagatggaagcaagagcggtttgattggaccgggactctttagtggccttgtttttggctctacttctgatcattctcagtctggagctagcgctgtctctgattgtactgattctgttgaacgaatacagtttgagaataggcagaagaacaagaagatgatgataccgatgcaggttctagtaccttcatcaatgaaatctccaagtaattcacatgaaggagaaacaaacatctatgacttcatggttccggaggagagagttcacggcggtgggctagtaatgtctttacttggtggctccattgatcgaaactgaaagccatttatggtaaaagtgtcacattctcagcaaaaacctgtgtaaagctgtaaaatgtgtgggaatctccgaatctgtttgtagccggttacgttatgctggatcaaaaactcaagatttgttggatattgttatgctggatcggtggtgaaaccacttcccggttgctaaataaataaacgtttttgttttataatctttttcactaaacggcagtatgggcctttagtgggcttcctttaagcgaccaatacaatcgtcgcaccggaatctactaccatttataggtttattcatgtaaaacctcggaaaatttgagagccacaacggtcaagagacaaaaacaacttgaagataaagggataaggaaggcttcctacatgatggacaacatttctttccacacaaattctcataataaaaatcttataatacaaatacttacgtcataatcattcaatctagtccccatgttttaaggtcctgtttcttgtctgatacaaat

SEQ ID NO: 93 - DNA, 1004 bp, Arabidopsis thaliana
  misc_feature (1)..(1004): Ceres Promoter YP0156
ttggtttgcattgtgaagatttgtattaactatagaacattgaattgatggtgttaagttcttacacaagcgtgcttctcggtttgaactgtttcttttgtatgttgaatcagagcttagtttataggaaccagagtatctacttagtcattctctgatgctaagtgctaaggttctacctagttgccctctaggcccttatgttattgataacttatgaagctatttgaacacttgattcttaggagacctaagttggtacagccagatagagtgtatgttcttgttctctatgtgacaggatcaagctgccacacatagttcaagggtatgctctgtgtgggtttgctcagattgaggacaaatctatacaaggaagtagagtctttgacattttgatgttgtatgataagaagaagaaaggagagtaataaagaaagagaaaagggaaacagaaacacgtgggagaacatcccaaagaggaagcacacgcggatcttcatgcaaagctccccgattctcccatgtggtccctttctccctttgtccccctcctctttcttcttttctcattttactcctttttttaccattatacaacgaatcttttttatcataattttttggttttggtttattttccaataacactttcttggttacttcccattctcactttttcatataagaaactcactttgggaaacttatgtttgagaatgacaagtctttttagagaaagtgatgtaacaaatctaaagtgattatataataaccttgcacaatgtttttgattttttgtaagattcgaatattaggtttattattcgtagggaataaacttactttcaaaagcgttcataagttaatactttcatatatgatcataagtacggacactattgttttttgtttgtttgtgtttattctaaaagaaagtagcttttaattgaaatgtcctcggaggcacagtttaaagttcgagtgtaacagtttctaaggca

SEQ ID NO: 94 - DNA, 1000 bp, Arabidopsis thaliana
  misc_feature (1)..(1000): Ceres Promoter YP0158
ttattagattaatagattgcattgcattgcttgtgctttcaatttacaaattgtctcccaactccatcgacacatctctttttgtgtatataagattcagacttgttatattttttttataaatatgttattagcatcttaagttaaattgattttttatatctgcattaaggattacacgactatatttgcgattgtgtgttggttaaaatataatttaggattgtctttaactacatttaggattatatgactatatttggttaaatataaaatctagctgtgattattagtattcaaaaataagtagcctaaccaattaaaacaacggctattggggcaaattagaacattttagtgtgtccaaaatataatggtcattaggtcatattcctcctagcttcatcgcagcataattgaatgattgccttatttagaagagcttttccactttcccaaaatctaggtgggatctttttgttttgaccttcatttttcttgtttaccatttttagctaaattatttacgattacaaaagatatcaaaagttggatcataatacaatttatagacttactgtagaaaattcgtatgtacaagtacaacaaattcttcataataaattttgaaaattctattacaaatgttgtaagaaatagaatttgaaatatatataaactaaggagaaaaaaaaagagaacatgcattgctctagtcagagtggaccaacatcaacgagataagataacataaaaaccaactcaccataactaaaaacatcccaagagatccaacgattcatatcaaacacaaaaacatcgaacgatcagatttaaaccatctctggtatctccaaaacacaaacacttttttttttcttttgtctgaatggaacaaaagcatgcgacatctctgtgtctttatcttctctctcctcttcttgaaaaactgaacctttaattctttcttcacatctcctttagctttctgaagctgcta

SEQ ID NO: 95 - DNA, 1005 bp, Arabidopsis thaliana
  misc_feature (1)..(1005): Ceres Promoter YP0188
gattggtatgaaatttcggagaccaacaaaaaaaactttattgagcttggagtgaagctatatatatggggcaagatcataatatgtttatatcggccttttcgttaactgaaaataatagttttgagaaatatatcaaatggtaaacagacatcatctttgaaaaataccatcaatgaagttaatattgttattggcatatggtttacccatcttaattttaatgcaaccaaacaaacaagaaacaaaaactgtataagatacaaggtgttttacgattttccgtcttaaaaccgaaatatttttgttcctacgactttaaacggactttgcttaagttgtgtgcatgtaagctcgtcgtccctcgattgtcatcaacattcaccaatatcagcctctatcacacgagtgaaggtggtgattcggcttaatgaaaacagagaaatatttcaatatgattcctattaaattttaaatcttttttctcaatctctagattttcattaaaagcatcatgatttttttccactatgttcatatatctctatcacagttttaggtacattgtagaaattggataagatacgtcatacgtctaacatgaatttggtctagcaaggaaggtttgagataataagtgaaaagaaaacacaagataataaattataatttataaatgctttatagtattgaaaaataagatgattttttttttttttaataccggattggctgatccacttatgatgactcaaatgttattaagtttcaagacaatttatgatgacacaaatcacaatgagtcaatagtagccacgaagccagaaaaaaaaaatgtactacaaaaagataatgatagtacaaaatgatacgtcgtactgccacatgtacgacacaactcgattaccaaaaagcagagccatccaaccataaaactcaaaacacacagattccactggcgtgtgctctcctcacttcactcgtccttgaaacttgaggtactga

SEQ ID NO: 96 - DNA, 1002 bp, Arabidopsis thaliana
  misc_feature (1)..(1002): Ceres Promoter YP0190
taaatagtgacattggtaagaagaaaaaaaacactattaaatagtgaaaaaatggtttataactctcttaattaacattacttattattgctagcacctaaaatctcccacaaaatatttgttgtaaaacacaaatttacaaaatgattttgtttttaaattagtaacacatgttcatatatacgttaataagaacataccctatatgattttatataaaaaaatttctttgagacgtcttattcttttttctttaataatatgcaattgtgagagtttggatttgaatggtagcattagaagcaaacttgaaccaaacatatttcatgaagtcaaacttgaaccaatgtgatcactaatcacagtgttcgcagtgtaaggcatcagaaaatagaagaagggacatagctatgaatcatataatcttgacacatgttttataggttttaggtgtgtatgctaacaaaaaatgagacagctttcttctaatagacttaatatttgggctaaatgtaccacagttgtgaatttcttacaaaaatgggccgagctacaaaaaactacaggcccactctcaactcttatcaaacgacagcgttttacttttttaaaagcacacactttttgtttggtgtcggtgacggtgagtttcgtccgctcttcctttaaattgaagcaacggttttgatccgatcaaatccaacggtgctgattacacaaagcccgagacgaaaacgttgactattaagttaggttttaatctcagccgttaatctacaaatcaacggttccctgtaaaacgaatcttccttccttcttcacttccgcgtcttctctctcaatcacctcaaaaaaatcgatttcatcaaaatattcacccgcccgaatttgactctccgatcatcgtctccgaatctagatcgacgagatcaaaaccctagaaatctaaatcggaatgagaaattgattttgatacgaattagggatctgtgtgttgaggac

SEQ ID NO: 97 - DNA, 995 bp, Arabidopsis thaliana
  misc_feature (1)..(995): Ceres Promoter YP0212
agtcgattggtacactcttaatttaattagagtaagagatcaacaaaaatatagaattttctttatatcgaagtgctacgaccttatatatatagaaaaaaaagcataggtgaatctctaaattgagattgtgctgtagtaaacatattaagtttttagtttttttaagaaatgaatctttttgttgattaattcaaactagtagtcattaagattccggagattccaatttagaaaagtcaaagattcaaagaacaagtccaggtccacatgttgaatccgattcatcatccactcatccttcatatcttcctccaccgtctccgcccaaaaaatcaataacaataaaaaatcctaaaaaaacatatttgattttgaaaaaactttatcatatattatattaattaaatagttatccgatgactcatcctatggtcagggccttgctgtctctgacgtccttaattatcattatttttaaatttgtctctctcagaaaattacgccacaatcttcctctttcccttttccgaaaacagctaatatttgtggacctaaactaaataacgtagcctctagattttatataattactaatactatatgctactacttgttattatttactccaatcatatatgataccaatcaagaatcactacataagtagaaaactttgcaatgagtccattaattaaaattaagaataaacttaaaattttatggtattttaagattccctttggattgtaatgacaagaaatcagcaaattagtcgtaactcgtaagaataaacaagatcaatttttactttctttacaaagattccgttgtaattttagaaatttttttttgtcactgtttttttatagattaatttatctgcatcaatccgattaagaagtgtacacatgggcatctatatatatctaacaggtaaaacgtgtatgtacatgcataaggttttacgtgcttctataaatatatgtggcagt

SEQ ID NO: 98 - DNA, 1024 bp, Arabidopsis thaliana
  misc_feature (1)..(1024): Ceres Promoter YP0214
ccagtcgattggcgcctcgcatgcctatcatatttaaccgtcaataatggatttggcggttttggtaggccgggtcaaccggattaaaagaaaacggtttggagtccttccttgcaattgaattttcacacattcgggttttgtgatttctctgtcataatgggcccggcacatatggttcataacccatgtgggcctatggtataatttttccaattaaaactattgttaggtcgataaaacaaaaaacaataaaaacgagtggaatacacataccaaaaagaatgtgatgaacattagtaattttattttgatggttaatgaaaaacaaaataaatgcatcttggcatcttccgttggaaagcgcaaatagggcagattttcagacagatatcactatgatggggggtgagagaaagaaaacgaggcgtacctaatgtaacactacttaattagtcgttagttataggactttttttttgtttgggcctagttataggatcataaggtaaaaatgaagaatgaatattagattagtaggagctaatgatggagttaagtatgcacgtgtaagaactgggaagtgaaacctcctgtatggtgaagaaactatacaacaaagccctttgttggtgtatacgtattaatttttattcttttatcacaagcgatacgtatcttaagacataataaatatatatcttactcataataaatatcttaagatatatatacagtatacacctgtatatatataataaataggcatatagtagaaattaatatgagttgttgttgttgcaaatatataaatcaatcaaaagatttaaaacccaccattcaatcttggtaagtaacgaaaaaaaagggaagcaagaagaaccacagaaaagggggctaacaactagacacgtagatcttcatctgcccgtccatctaacctaccacactctcatcttctttttcccgtgtcagtttgttatataagctctcactctccggtatatttccccattgcactgga

SEQ ID NO: 99 - DNA, 911 bp, Arabidopsis thaliana
  misc_feature (1)..(911): Ceres Promoter YP0263
atctagctgtggattccaccaaaattctggcagggccatgatctaaaaactgagactgcgcgtgttgttttgcagtgatttgtatttcatatttgcaccatcctacacagtccacttggtatcgtaaccaaacataaggagaacctaattacattattgttttaatttcgtcaaactggtttttaccttttagttacatagttgattcttcatttgttttagtagttatggagcacaataatgtgcaacaaagaaagatcatagtggattaatatgttgagaggtcagaaattcttggttaacaaaaaaaagttacaaggactgagattttgggtgggagaaagccatagcttttaaaacatgattgaacttaaaagtgatgttatggtttgaggggaaaaaggttgatgtcaactaagatagttgaagtaatgtcttaaactaaagtaaaccaccggtccaaccgtggtccggaagcatctctggtatgatttatcctaaaaatcaaaatagtagaaacatactttaaatatatacattgatcggacgaaaattgtaaactagtatagtttcaaaaactagttgaacaggttatgtaccttaaacatttatttcaaacttaaacactaaagaacatatatgaatagaagtttatataaattactatatatctaccataaatctcttataattatgatgtcacgatgaggaagtgttgaaacgttaaaatgccaaaatataagcatgcgacggaattttggcagaagattgtagagttgtaatctgtcgcaatcattactcgtgctagcatttttcattttcccttcatttgtggataacgcacgatataacattctacacaccaacaagattctataaaaacgcaaaggttgtctccatagaatatcgtc

SEQ ID NO: 100 - DNA, 999 bp, Arabidopsis thaliana
  misc_feature (1)..(999): Ceres Promoter YP0275
aaacattaatatgtagtaactatgggcgtatgctttactttttaaaatgggcctatgctataattgaatgacaaggattaaacaactaataaaattgtagatgggttaagatgacttatttttttacttaccaatttataaatgggcttcgatgtactgaaatatatcgcgcctattaacgaggccattcaacgaatgttttaagggccctatttcgacattttaaagaacacctaggtcatcattccagaaatggatattataggatttagataatttcccacgtttggtttatttatctattttttgacgttgaccaacataatcgtgcccaaccgtttcacgcaacgaatttatatacgaaatatatatatttttcaaattaagataccacaatcaaaacagctgttgattaacaaagagattttttttttttggttttgagttacaataacgttagaggataaggtttcttgcaacgattaggaaatcgtataaaataaaatatgttataattaagtgttttattttataatgagtattaatataaataaaacctgcaaaaggatagggatattgaataataaagagaaacgaaagagcaattttacttctttataattgaaattatgtgaatgttatgtttacaatgaatgattcatcgttctatatattgaagtaaagaatgagtttattgtgcttgcataatgacgttaacttcacatatacacttattacataacatttatcacatgtgcgtctttttttttttttactttgtaaaatttcctcacttttaagacttttataacaattactagtaaaataaagttgcttggggctacaccctttctccctccaacaactctatttatagataacattatatcaaaatcaaaacatagtccctttcttctataaaggttttttcacaaccaaatttccattataaatcaaaaaataaaaacttaattagtttttacagaagaaaagaaaaca

SEQ ID NO: 101 - DNA, 981 bp, Arabidopsis thaliana
  misc_feature (1)..(981): Ceres Promoter YP0285
gggattatatatgatagacgattgtatttgcgggacattgagatgtttccgaaaatagtcatcaaatatcaaaccagaatttgatgtgaaaacactaattaaaacatataattgacaactagactatatcatttgttaagttgagcgttgaaagaaaatgaaagagtgtagactgtagtacgtatgagtttcccaaaagatggtgcttgaatattattgggaagagactttggttggttcggttgaatgaagatttttacctgccatgttgatagagaaaggcaaataaatgtaggggtcgatgtctaacgtaaagactggatcaaccaagagtcctcctcctcgtcttcaccaaaaaaaaagagtcctcctcgtggaaacttatttcttctccagccaagatctcatctcatctcttcactctatgaaatataaaggaatcttatggtttttctaaaaactatagtacgtctatataccaaaggaaacaatataaaatcagttaatctgataaattttgagtaaataataaagttaactttgtacttacctatatcaaactaattcacaaaataaagtaataataacaaagaatttttagtagatccacaatatacacacacactatgagaaatcataatagagaattttaatgattttgtctaactcatagcaacaagtcgctttggccgagtggttaaggcgtgtgcctgctaagtacatgggctctgcccgcgagagttcgaatctctcaggcgacgtttcttttgttttcggccataaaggaaaaagcccaattaacacgtctcgcttataagcccataaagcaaacaatgggctgtctctgtctcactcacacacgcgttttcctactttttgactatttttataaccggcgggtctgacttaattagggttttctttaataatcagacactctctcactcgtttcgtcaacattgaacacagacaaaaccgcgt

SEQ ID NO: 102 - DNA, 996 bp, Arabidopsis thaliana
  misc_feature (1)..(996): Ceres Promoter YP0286
gaaaacaatcataggttacgctattatcatcgaaaggtatgtgatgcatattcccattgaaccagatttccatatattttatttgtaaagtgataatgaatcacaagatgattcaatattaaaaatgggtaactcactttgacgtgtagtacgtggaagaatagttagctatcacgcatacatatatctatgaataagtgtgtatgacataagaaactaaaatatttacctaaagtccagttactcatactgatttcatgcatatatgtattatttatttatttttaataaagaagcgattggtgttttcatagaaatcatgatagattgataggtatttcagttccacaaatctagatctgtgtgctatacatgcatgtattaattttttccccttaaatcatttcagttgataatattgctctttgttccaactttagaaaaggtatgaaccaacctgacgattaacaagtaaacattaattaatctttatatgagataaaaccgaggatatatatgattgtgttgctgtctattgatgatgtgtcgatattatgcttgttgtaccaatgctcgagccgagcgtgatcgatgccttgacaaactatatatgtttcccgaattaattaagttttgtatcttaattagaataacatttttatacaatgtaatttctcaagcagacaagatatgtatcctatattaattactatatatgaattgccgggcacctaccaggatgtttcaaatacgagagcccattagtttccacgtaaatcacaatgacgcgacaaaatctagaatcgtgtcaaaactctatcaatacaataatatatatttcaagggcaatttcgacttctcctcaactcaatgattcaacgccatgaatctctatataaaggctacaacaccacaaaggatcatcagtcatcacaaccacattaactcttcaccactatctctcaatctctcgtttcatttcttgacgcgtgaaaa

SEQ ID NO: 103 - DNA, 1000 bp, Arabidopsis thaliana
  misc_feature (1)..(1000): Ceres Promoter YP0337
taatttttttatttttggaactaacacttattagtttaggtttccatcacctatttaattcgtaattcttatacatgcatataatagagatacatatatacaaatttatgatcatttttgcacaacatgtgatctcattcattagtatgcattatgcgaaaacctcgacgcgcaaaagacacgtaatagctaataatgttactcatttataatgattgaagcaagacgaaaacaacaacatatatatcaaattgtaaactagatatttcttaaaagtgaaaaaaaacaaagaaatataaaggacaattttgagtcagtctcttaatattaaaacatatatacataaataagcacaaacgtggttacctgtcttcatgcaatgtggactttagtttatctaatcaaaatcaaaataaaaggtgtaatagttctcgtcatttttcaaattttaaaaatcagaaccaagtgatttttgtttgagtattgatccattgtttaaacaatttaacacagtatatacgtctcttgagatgttgacatgatgataaaatacgagatcgtctcttggttttcgaattttgaactttaatagttttcttttttagggaaactttaatagttgtttatcataagattagtcacctaatggttacgttgcagtaccgaaccaattttttacccttttttctaaatgtggtcgtggcataatttccaaaagagatccaaaacccggtttgctcaactgataagccggtcggttctggtttgaaaaacaagaaataatctgaaagtgtgaaacagcaacgtgtctcggtgtttcatgagccacctgccacctcattcacgtcggtcattttgtcgtttcacggttcacgctctagacacgtgctctgtccccaccatgactttcgctgccgactcgcttcgctttgcaaactcaaacatgtgtgtatatgtaagtttcatcctaataagcatctcttaccacattaattaaaaa

SEQ ID NO: 104 - DNA, 1000 bp, Arabidopsis thaliana
  misc_feature (1)..(1000): Ceres Promoter YP0356
ttagttcattgaaacgtcaactttttacttgcaaccactttgtaggaccattaactgcaaaataagaattctctaagcttcacaaggggttcgtttggtgctataaaaacattgttttaagaactggtttactggttctataaatctataaatccaaatatgaagtatggcaataataataacatgttagcacaaaaaatactcattaaattcctacccaaaaaaaatctttatatgaaactaaaacttatatacacaataatagtgatacaaagtaggtcttgatattcaactattcgggattttctggtttcgagtaattcgtataaaaggtttaagatctattatgttcactgaaatcttaactttgttttgtttccagttttaactagtagaaattgaaatttttaaaaattgttacttacaataaaatttgaatcaatatccttaatcaaaggatcttaagactagcacaattaaaacatataacgtagaatatctgaaataactcgaaaatatctgaactaagttagtagttttaaaatataatcccggtttggaccgggcagtatgtacttcaatacttgtgggttttgacgattttggatcggattgggcgggccagccagattgatctattacaaatttcacctgtcaacgctaactccgaacttaatcaaagattttgagctaaggaaaactaatcagtgatcacccaaagaaaacattcgtgaataattgtttgctttccatggcagcaaaacaaataggacccaaataggaatgtcaaaaaaaagaaagacacgaaacgaagtagtataacgtaacacacaaaaataaactagagatattaaaaacacatgtccacacatggatacaagagcatttaaggagcagaaggcacgtagtggttagaaggtatgtgatataattaatcggcccaaatagattggtaagtagtagccgtctatatcatccatactcatcataacttcaacct

SEQ ID NO: 105 - DNA, 1000 bp, Arabidopsis thaliana
  misc_feature (1)..(1000): Ceres Promoter YP0374
aagacacccgtaaatgttgtcatgtagaagaaactagaaacgttaaacgcatcaaatcaagaaattaaattgaaggtaatttttaacgccgcctttcaaatattcttcctaggagaggctacaagacgcgtatttctttcgaattctccaaaccattaccattttgatatataataccgacatgccgttgataaagtttgtatgcaaatcgttcattgggtatgagcaaatgccatccattggttcttgtaattaaatggtccaaaaatagtttgttcccactactagttactaatttgtatcactctgcaaaataatcatgatataaacgtatgtgctatttctaattaaaactcaaaagtaatcaatgtacaatgcagagatgaccataaaagaacattaaaacactacttccactaaatctatggggtgccttggcaaggcaattgaataaggagaatgcatcaagatgatatagaaaatgctattcagtttataacattaatgttttggcggaaaattttctatatattagacctttctgtaaaaaaaaaaaaatgatgtagaaaatgctattatgtttcaaaaatttcgcactagtataatacggaacattgtagtttacactgctcattaccatgaaaaccaaggcagtatataccaacattaataaactaaatcgcgatttctagcacccccattaattaattttactattatacattctctttgcttctcgaaataataaacttctctatatcattctacataataaataagaaagaaatcgacaagatctaaatttagatctattcagctttttcgcctgagaagccaaaattgtgaatagaagaaagcagtcgtcatcttcccacgtttggacgaaataaaacataacaataataaaataataaatcaaatatataaatccctaatttgtctttattactccacaattttctatgtgtatatatatacccacctctctcttgtgtatttg

SEQ ID NO: 106 - DNA, 998 bp, Arabidopsis thaliana
  misc_feature (1)..(998): Ceres Promoter YP0377
tataaaccattcctataacaccatatttaaacataacaatgaattgcttggatttcaaactttattaaatttggattttaaattttaatttgattgaattatacccccttaattggataaattcaaatatgtcaactttttttttgtaagatttttttatggaaaaaaaaattgattattcactaaaaagatgacaggttacttataatttaatatatgtaaaccctaaaaagaagaaaatagtttctgttttcactttaggtcttattatctaaacttctttaagaaaatcgcaataaattggtttgagttctaactttaaacacattaatatttgtgtgctatttaaaaaataatttacaaaaaaaaaaacaaattgacagaaaatatcaggttttgtaataagatatttcctgataaatatttagggaatataacatatcaaaagattcaaattctgaaaatcaagaatggtagacatgtgaaagttgtcatcaatatggtccacttttctttgctctataacccaaaattgaccctgacagtcaacttgtacacgcggccaaacctttttataatcatgctatttatttccttcatttttattctatttgctatctaactgatttttcattaacatgataccagaaatgaatttagatggattaattcttttccatccacgacatctggaaacacttatctcctaattaaccttactttttttttagtttgtgtgctccttcataaaatctatattgtttaaaacaaaggtcaataaatataaatatggataagtataataaatctttattggatatttctttttttaaaaaagaaataaatcttttttggatattttcgtggcagcatcataatgagagactacgtcgaaaccgctggcaaccacttttgccgcgtttaatttctttctgaggcttatataaatagatcaaaggggaaagtgagatataatacagacaaaacaagagaaaaga

SEQ ID NO: 107 - DNA, 999 bp, Arabidopsis thaliana
  misc_feature (1)..(999): Ceres Promoter YP0380
acaagtaccattcacttttttacttttcaatgtatacaatcatcatgtgataaaaaaaaaaatgtaaccaatcaacacactgagatacggccaaaaaatggtaatacataaatgtttgtaggttttgtaatttaaatactttagttaagttatgattttattatttttgcttatcacttatacgaaatcatcaatctattggtatctcttaatcccgctttttaatttccaccgcacacgcaaatcagcaaatggttccagccacgtgcatgtgaccacatattgtggtcacagtactcgtcctttttttttcttttgtaatcaataaatttcaatcctaaaacttcacacattgagcacgtcggcaacgttagctcctaaatcataacgagcaaaaaagttcaaattagggtatatgatcaattgatcatcactacatgtctacataattaatatgtattcaaccggtcggtttgttgatactcatagttaagtatatatgtgctaattagaattaggatgaatcagttcttgcaaacaactacggtttcatataatatgggagtgttatgtacaaaatgaaagaggatggatcattctgagatgttatgggctcccagtcaatcatgttttgctcgcatatgctatcttttgagtctcttcctaaactcatagaataagcacgttggttttttccaccgtcctcctcgtgaacaaaagtacaattacattttagcaaattgaaaataaccacgtggatggaccatattatatgtgatcatattgcttgtcgtcttcgttttcttttaaatgtttacaccactacttcctgacacgtgtccctattcacatcatccttgttatatcgttttacttataaaggatcacgaacaccaaaacatcaatgtgtacgtcttttgcataagaagaaacagagagcattatcaattattaacaattacacaagacagcgagattgtaaaagagtaagagagagag

SEQ ID NO: 108 - DNA, 1000 bp, Arabidopsis thaliana
  misc_feature (1)..(1000): Ceres Promoter YP0381
cacggtcaaagtattgctaacatggtcattacattgaaaaagaaaattaattgtctttactcatgtttattctatacaaataaaaatattaaccaaccatcgcactaacaaaatagaaatcttattctaatcacttaattgttgacaattaaatcattgaaaaatacacttaaatgtcaaatattcgttttgcatacttttcaatttaaatacatttaaagttcgacaagttgcgtttactatcatagaaaactaaatctcctaccaaagcgaaatgaaactactaaagcgacaggcaggttacataacctaacaaatctccacgtgtcaattaccaagagaaaaaaagagaagataagcggaacacgtggtagcacaaaaaagataatgtgatttaaattaaaaaacaaaaacaaagacacgtgacgacctgacgctgcaacatcccaccttacaacgtaataaccactgaacataagacacgtgtacgatcttgtctttgttttctcgatgaaaaccacgtgggtgctcaaagtccttgggtcagagtcttccatgattccacgtgtcgttaatgcaccaaacaagggtactttcggtattttggcttccgcaaattagacaaaacagctttttgtttgattgatttttctcttctctttttccatctaaattctctttgggctcttaatttctttttgagtgttcgttcgagatttgtcggagattttttcggtaaatgttgaaattttgtgggatttttttttatttctttattaaacttttttttattgaatttataaaaagggaaggtcgtcattaatcgaagaaatggaatcttccaaaatttgatattttgctgttttcttgggatttgaattgctctttatcatcaagaatctgttaaaatttctaatctaaaatctaagttgagaaaaagagagatctctaatttaaccggaattaatattctccgaccgaagttattatgttgcaggct

SEQ ID NO: 109 - DNA, 999 bp, Arabidopsis thaliana
  misc_feature (1)..(999): Ceres Promoter YP0384
tttaaaaaattggataaaacaccgataaaaattcacatttgcaaattttattcagtcggaatatatatttgaaacaagttttgaaatccattggacgattaaaattcattgttgagaggataaatatggatttgttcatctgaaccatgtcgttgattagtgattgactaccatgaaaaatatgttatgaaaagtataacaacttttgataaatcacatttattaacaataaatcaagacaaaatatgtcaacaataatagtagtagaagatattaattcaaattcatccgtaacaacaaaaaatcataccacaattaagtgtacagaaaaaccttttggatatatttattgtcgcttttcaatgattttcgtgaaaaggatatatttgtgtaaaataagaaggatcttgacgggtgtaaaaacatgcacaattcttaatttagaccaatcagaagacaacacgaacacttctttattataagctattaaacaaaatcttgcctattttgcttagaataatatgaagagtgactcatcagggagtggaaaatatctcaggatttgcttttagctctaacatgtcaaactatctagatgccaacaacacaaagtgcaaattcttttaatatgaaaacaacaataatatttctaatagaaaattaaaaagggaaataaaatatttttttaaaatatacaaaagaagaaggaatccatcatcaaagttttataaaattgtaatataatacaaacttgtttgcttccttgtctctccctctgtctctctcatctctcctatcttctccatatatacttcatcttcacacccaaaactccacacaaaatatctctccctctatctgcaaattttccaaagttgcatcctttcaatttccactcctctctaatataattcacattttcccactattgctgattcatttttttttgtgaattatttcaaacccacataaaaaaatctttgtttaaatttaaaacca

SEQ ID NO: 110 - DNA, 998 bp, Arabidopsis thaliana
  misc_feature (1)..(998): Ceres Promoter YP0385
actcaacaataggacaagccaaaaaaattccaattattgtgttactctattcttctaaatttgaacactaatagactatgacatatgagtatataatgtgaagtcttaagatattttcatgtgggagatgaataggccaagttggagtctgcaaacaagaagctcttgagccacgacataagccaagttgatgaccgtaattaatgaaactaaatgtgtgtggttatatattagggacccatggccatatacacaatttttgtttctgtcgatagcatgcgtttatatatatttctaaaaaaactaacatatttactggatttgagttcgaatattgacactaatataaactacgtaccaaactacatatgtttatctatatttgattgatcgaagaattctgaactgttttagaaaatttcaatacacttaacttcatcttacaacggtaaaagaaatcaccactagacaaacaatgcctcataatgtctcgaaccctcaaactcaagagtatacattttactagattagagaatttgatatcctcaagttgccaaagaattggaagcttttgttaccaaacttagaaacagaagaagccacaaaaaaagacaaagggagttaaagattgaagtgatgcatttgtctaagtgtgaaaggtctcaagtctcaactttgaaccataataacattactcacactccctttttttttctttttttttcccaaagtaccctttttaattccctctataacccactcactccattccctctttctgtcactgattcaacacgtggccacactgatgggatccacctttcctcttacccacctcccggtttatataaacccttcacaacacttcatcgctctcaaaccaactctctcttctctcttctctcctctcttctacaagaagaaaaaaaacagagcctttacacatctcaaaatcgaacttactttaaccaccaaatactgattgaacacacttgaaa

SEQ ID NO: 111 - DNA, 1000 bp, Arabidopsis thaliana
  misc_feature (1)..(1000): Ceres Promoter YP0396
catagtaaaagtgaatttaatcatactaagtaaaataagataaaacatgttatttgaatttgaatatcgtgggatgcgtatttcggtatttgattaaaggtctggaaaccggagctcctataacccgaataaaaatgcataacatgttcttccccaacgaggcgagcgggtcagggcactagggtcattgcaggcagctcataaagtcatgatcatctaggagatcaaattgtatgtcggccttctcaaaattacctctaagaatctcaaacccaatcatagaacctctaaaaagacaaagtcgtcgctttagaatgggttcggtttttggaaccatatttcacgtcaatttaatgtttagtataatttctgaacaacagaattttggatttatttgcacgtatacaaatatctaattaataaggacgactcgtgactatccttacattaagtttcactgtcgaaataacatagtacaatacttgtcgttaatttccacgtctcaagtctataccgtcatttacggagaaagaacatctctgtttttcatccaaactactattctcactttgtctatatatttaaaattaagtaaaaaagactcaatagtccaataaaatgatgaccaaatgagaagatggttttgtgccagattttaggaaaagtgagtcaaggtttcacatctcaaatttgactgcataatcttcgccattaacaacggcattatatatgtcaagccaattttccatgttgcgtacttttctattgaggtgaaaatatgggtttgttgattaatcaaagagtttgcctaactaatataactacgactttttcagtgaccattccatgtaaactctgcttagtgtttcatttgtcaacaatattgtcgttactcattaaatcaaggaaaaatatacaattgtataattttcttatattttaaaattaattttgatgtattacccctttataaataggctatcgctacaacaccaataac

SEQ ID NO: 112 - DNA, 1514 bp, Arabidopsis thaliana
  misc_feature (1)..(1514): Ceres Promoter p13879
tttcgatcctcttcttttttaggtttcttgatttgatgatcgccgccagtagagccgtcgtcggaagtttcagagattaaaaccatcaccgtgtgagttggtagcgaattaacggaaagtctaagtcaagattttttaaaaagaaatttatgtgtgaaaagaagccgttgtgtatatttatataatttagaaaatgtttcatcattttaattaaaaaattaataatttgtagaagaaagaagcattttttatacataaatcatttaccttctttactgtgtttttcttcacttacttcatttttacttttttacaaaaaagtgaaaagtaaattacgtaattggtaacataaattcactttaaatttgcatatgttttgttttcttcggaaactatatcgaaaagcaaacggaaagaacttcacaaaaaaccctagctaactaaagacgcatgtgttcttcttattcttcatatatcctctgtttcttgtgttctgttttgagtcttacattttcaatatctgactctgattactatatctaaaagggaacatgaagaacttgagaccatgttaaactgtacaatgccttcaaacatggctaactaaagatacattagatggctttacagtgtgtaatgcttattatctttaggttttttaaatcccttgtattaagttatttaccaaattatgttcttgtactgcttattggcttggttgttgtgtgctttgtaaacaacacctttggctttatttcatcctttgtaaacctactggtctttgttcagctcctcttggaagtgagtttgtatgcctggaacgggttttaatggagtgtttatcgacaaaaaaaaaatgtagcttttgaaatcacagagagtagttttatattcaaattacatgcatgcaactaagtagcaacaaagttgatatggccgagttggtctaaggcgccagattaaggttctggtccgaaagggcgtgggttcaaatcccactgtcaacattctctttttctcaaattaatatttttctgcctcaatggttcaggcccaattatactagactactatcgcgactaaaatagggactagccgaattgatccggcccagtatcagttgtgtatcaccacgttatttcaaatttcaaactaagggataaagatgtcatttgacatatgagatatttttttgctccactgagatatttttctttgtcccaagataaaatatcttttctcgcatcgtcgtctttccatttgcgcattaaaccaaaaagtgtcacgtgatatgtccccaaccactacgaattttaactacagatttaaccatggttaaaccagaattcacgtaaaccgactctaaacctagaaaatatctaaaccttggttaatatctcagcccccttataaataacgagacttcgtctacatcgttctacacatctcactgctcactactctcactgtaatcccttagatcttcttttcaaatttcaccattgcactggatg

SEQ ID NO: 113 - DNA, 1954 bp, Arabidopsis thaliana
  misc_feature (1)..(1954): Ceres Promoter p326
gtgggtaaaagtatccttctttgtgcatttggtatttttaagcatgtaataagaaaaaccaaaatagacggctggtatttaataaaaggagactaatgtatgtatagtatatgatttgtgtggaatataataaagttgtaaaatatagatgtgaagcgagtatctatcttttgactttcaaaggtgatcgatcgtgttctttgtgatagttttggtcgtcggtctacaagtcaacaaccaccttgaagttttcgcgtctcggtttcctcttcgcatctggtatccaatagcatacatataccagtgcggaaaatggcgaagactagtgggcttgaaccataaggtttggccccaatacggattccaaacaacaagcctagcgcagtcttttgggatgcataagactaaactgtcgcagtgatagacgtaagatatatcgacttgattggaatcgtctaagctaataagtttaccttgaccgtttatagttgcgtcaacgtccttatggagattgatgcccatcaaataaacctgaaaatccatcaccatgaccaccataaactcccttgctgccgctgctttggcttgagcaaggtgtttccttgtaaagctccgatctttggataaagtgttccactttttgcaagtagctctgacccctctcagagatgtcaccggaatcttagacagaacctcctctgccaaatcacttggaagatcggacaatgtcatcatttttgcaggtaatttctccttcgttgctgctttggcttgagcacggtgcttctttgtaaagctccgatctttggataagagcggatcggaatcctctaggaggtgccagtcccttgacctattaatttatagaaggttttagtgtattttgttccaatttcttctctaacttaacaaataacaactgcctcatagtcatgggcttcaaattttatcgcttggtgtatttcgttatttgcaaggccttggcccattttgagcccaataactaaatctagccttttcagaccggacatgaacttcgcatattggcgtaactgtgcagttttacctttttcggatcagacaagatcagatttagaccacccaacaatagtcagtcatatttgacaacctaagctagccgacactactaaaaagcaaacaaaagaagaattctatgttgtcattttaccggtggcaagtggacccttctataaaagagtaaagagacagcctgtgtgtgtataatctctaattatgttcaccgacacaatcacacaaacccttctctaatcacacaacttcttcatgatttacgacattaattatcattaactctttaaattcactttacatgctcaaaaatatctaatttgcagcattaatttgagtaccgataactattattataatcgtcgtgattcgcaatcttcttcattagatgctgtcaagttgtactcgcacgcggtggtccagtgaagcaaatccaacggtttaaaaccttcttacatttctagatctaatctgaaccgtcagatatctagatctcattgtctgaacacagttagatgaaactgggaatgaatctggacgaaattacgatcttacaccaaccccctcgacgagctcgtatatataaagcttatacgctcctccttcaccttcgtactactactaccaccacatttctttagctcaaccttcattactaatctccttttaaggtatgttcacttttcttcgattcatactttctcaagattcctgcatttctgtagaatttgaaccaagtgtcgatttttgtttgagagaagtgttgatttatagatctggttattgaatctagattccaatttttaattgattcgagtttgttatgtgtgtttatactacttctcattgatcttgtttgatttctctgctctgtattaggtttctttcgtgaatcagatcggaa

SEQ ID NO: 114 - DNA, 2016 bp, Arabidopsis thaliana
  misc_feature (1)..(2016): Ceres Promoter p32449
gatcggccttcttcaggtcttctctgtagctctgttacttctatcacagttatcgggtatttgagaaaaaagagttagctaaaatgaatttctccatataatcatggtttactacaggtttacttgattcgcgttagctttatctgcatccaaagttttttccatgatgttatgtcatatgtgataccgttactatgtttataactttatacagtctggttcactggagtttctgtgattatgttgagtacatactcattcatcctttggtaactctcaagtttaggttgtttgaattgcctctgttgtgatacttattgtctattgcatcaatcttctaatgcaccaccctagactatttgaacaaagagctgtttcattcttaaacctctgtgtctccttgctaaatggtcatgctttaatgtcttcacctgtctttctcttctatagatatgtagtcttgctagatagttagttctacagctctcttttgtagtcttgttagagagttagttgagatattacctcttaaaagtatccttgaacgctttccggttatgaccaatttgttgtagctccttgtaagtagaacttactgggaccagcgagacagtttatgtgaatgttcatgcttaagtgtcgaacgtatctatctctactatagctctgtagtcttgttagacagttagttttatatctccatttttttgtagtcttgctagttgagatattacctcttctcttcaaagtatccttgaacgctcaccggttatgaaatctctacactatagctctgtagtcttgctagatagttagttctttagctctctttttgtagcctagttctttagctctccttttgtagccttgctacagagtaagatgggatattacctccttgaacgctctccggttatgaccaatttgttgtagctccttgtaagtagaacttaggatagagtgagtcaactttaagaaagaacctagtatgtggcataaccagattgcaggctctgtctcggctacagtaacgtaactctatagctctttgttttgttcagaaagaaccagtgattggatgattcgtccttagaaactggacctaacaacagtcattggctttgaaatcaagccacaacaatgcctatatgaaccgtccatttcatttatccgtttcaaaccagcccattacatttcgtcccattgataaccaaaagcggttcaatcagattatgttttaattttaccaaattctttatgaagtttaaattatactcacattaaaaggattattggataatgtaaaaattctgaacaattactgattttggaaaattaacaaatattctttgaaatagaagaaaaagcctttttccttttgacaacaacatataaaatcatactcccattaaaaagattttaatgtaaaattctgaatataagatattttttacaacaacaaccaaaaatatttatttttttccttttttacagcaacaagaaggaaaaacttttttttttgtcaagaaaaggggagattatgtaaacagataaaacagggaaaataactaaccgaactctcttaattaacatcttcaaataaggaaaattatgatccgcatatttaggaagatcaatgcattaaaacaacttgcacgtggaaagagagactatacgctccacacaagttgcactaatggtacctctcacaaaccaatcaaaatactgaataatgccaacgtgtacaaattagggttttacctcacaaccatcgaacattctcgaaacattttaaacagcctggcgccatagatctaaactctcatcgaccaatttttgaccgtccgatggaaactctagcctcaacccaaaactctatataaagaaatcttttccttcgttattgcttaccaaatacaaaccctagccgccttattcgtcttcttcgttctctagttttttcctcagtctctgttcttagatcccttgtagtttccaaatcttccgataaggcct

SEQ ID NO: 115 - DNA, 857 bp, Arabidopsis thaliana
  misc_feature (1)..(857): Ceres Promoter PD1367
  misc_feature (116)..(116): n is a, c, t, g, unknown, or other
  misc_feature (136)..(136): n is a, c, t, g, unknown, or other
  misc_feature (154)..(154): n is a, c, t, g, unknown, or other
  misc_feature (159)..(159): n is a, c, t, g, unknown, or other
  misc_feature (168)..(168): n is a, c, t, g, unknown, or other
  misc_feature (172)..(172): n is a, c, t, g, unknown, or other
  misc_feature (175)..(175): n is a, c, t, g, unknown, or other
  misc_feature (679)..(679): n is a, c, t, g, unknown, or other
  misc_feature (680)..(680): n is a, c, t, g, unknown, or other
  misc_feature (686)..(686): n is a, c, t, g, unknown, or other
  misc_feature (724)..(724): n is a, c, t, g, unknown, or other
  misc_feature (737)..(737): n is a, c, t, g, unknown, or other
ttggaattaattctgcggccatggggctgcaggaattcgatggcccgatcggccacagttttcttttctcatcttacaacaagtttccaggaggatagagacataaacgaagctcnggattgtatcgttctttttnagcttttattcacatccngaaangtcctgtangttntangattctgttatcttgcggttttgagttaatcagaaacagagtaatcaatgtaatgttgcaggctagatctttcatctttggaaatttgtttttttctcatgcaatttctttagcttgaccatgagtgactaaaagatcaatcagtagcaatgatttgatttggctaagagacatttgtccacttggcatcttgatttggatggttacaacttgcaagacccaattggatacttgctatgacaactccaactcaagagtgtcgtgtaactaagaaccttgactaatttgtaatttcaatcccaagtcatgttactatatgtttttttgtttgtattattttctctcctacaattaagctctttgacgtacgtaatctccggaaccaactcctatatccaccatttactccacgttgtctccaattattggacgttgaaacttgacacaacgtaaacgtatctacgtggttgattgtatgtacatatgtacaaacgtacacctttnnctcctnctttcacttcatcacttggcttgtgaattcattaattncctgcgaaggccntgcagggccatcaccactgcagtggaacaatgaagactaatctttttctctttctcatcttttcacttctcctatcattatcctcggccgaattcagtaaaggagaagaacttttcactg

SEQ ID NO: 116 - DNA, 999 bp, Arabidopsis thaliana
  misc_feature (1)..(999): Ceres Promoter YP0087
tgaattgagtaaaatgtgttttcaaacagttaggtggtagaaggtaaaggtaataacatcatgatcttactaaaagaattgttgcatactaactatcaatattctcaacaacataatataatgtttttttaggtaattttccattttaattttttgtgattaaacaattaaacaactcgaatgatgatgataaaaaaaaaaaattaacaactcgaataagttaaagtagcaatacacatgtcgttcaattcaaccaataaagtaagacttatatttttaagaagttgactaatagcttaataagttggaaaacttgtgtagtttcttaattcccacgtgcagtaagaaataaaaatgaaaaaaattattatatccttcccactctgcgacttttcttttattttatcaaatattaaaaagattcatatcacagtttacacattgaaatcataaacgataattatgtattttgtaataaaaagttagttctgaagctcatactttggatagtcgctagtcgctaatatgctccttgtaataattaaagtcactacgacgcacgtcaaagccgatatttagggcttaattgatgcgtgtttttcttttcatataatagtaatataaattagtactaataaagtatgatggatggttgagacagaaaagaaaaaagatgactgtatggtcatcattacaaagaagaatgtattcttcatgttcttaagaataataaaatgtcacttgtaaatcaagttggtaagcattttgagaactttgttcgatgcaacgtatgatgatttatgtagacaaaagataaaaccgtatcttcaactattgccaagaaaagataaaacctaatctagtcagtctctcaacataaatacaacccaatagccaaactgtgtccaattcggagagaaactaaactaaaacaaaacacaaaagcccaacataagcccaataaaacccattttataaacagaacattactaacactca

SEQ ID NO: 117 - DNA, 1000 bp, Arabidopsis thaliana
  misc_feature (1)..(1000): Ceres Promoter YP0093
atgatgaacattctacatatataattattatgtttaagcacttagacagcataaattctttctaattatataaatctaaccttgttacattgtacatctataaattacttgaagaaataacgagttctatttctttttaaaaattaaaaatactataccatatctcagtgattaagttgaaccaaaaggtacggaggagaaacaagcatttgattcttccttattttattttattcatctctcactaatgatggtggagaaaaaaagaaaatacctaacaaacaaatatatattgtcatacaaaaatatttctatatttttagttaattagtttatattcctcacttttcagggcttatataagaaagtgagcaaacacaaatcaaaatgcagcagcaaatactatcatcacccatctccttagttctattttataattcctcttctttttgttcatagctttgtaattatagtcttatttctctttaaggctcaataagaggaggtactattactacacttctctctacttttacttgtattttagcattaaaatcctaaaatccgttttaaattcaaaaataaacttagagatgtttaatctcgattcggtttttcggctttaggagaataattatatgaaattagtatggatatctttactagtttccattcaaatgattctgatttcaatctaatactctcactctttaattaaactatatgtagtgtaatttcacactgttaaatttctaccatgtcatgtatattagagttgcatagaaaattgtaaaacatccatttgaattcgaatgaaacaaaatgttttaaaataaaattttggtttttaaaagaaaaatctaaaactgaattatatcgtttaaccaagttgtaaaagtcataaaacgtagtatcttgtaaatcgctcttccacggtccaaatagacttctagtaataaacaagtaaaactaattttggtttcttactaattttcacaga

SEQ ID NO: 118 - DNA, 999 bp, Arabidopsis thaliana
  misc_feature (1)..(999): Ceres Promoter YP0108
ttagctgaaccaggaaattgatctcttataccagtttccgggtttagattggtttgatggcgatttgattaaacccccgaaattttatgtcgtagttgtgcatagtattattattctttgcggacaatagacgtatcgggaccaagttctgtagcaaaattgtataagcttaagtttgatgaaatttaaaggtaatcactaaaacccaaatgggacaataaaccggtgaagatttagagtttttaattttgactcatgaatctggagaaagagccctcgttaaaaggagtgaatcaatccataggggaaaaagttttgtctttttaaaaactaaagaaccaaaccttaatagaagcagctcaatgtgtgacaactttccactggcactaagataaagtgactagcgatgagtgcaattattgaaatagtagatggtaaatattacatacaagagtaaaaatatctttatgtcaatgcttaattcagtgtttctggttaacaagagaaacttctctaactttcgtaattgggtcttataaaattttatgcaattatgattttacccttttactacttttcattagctttcacgaatctattttgacaagagaaatcattagaggtaaacatgctttttggtcaagggccttaacagttccaccaatcaagctcaaaagttgtacttaaccgacatcttctgtgaaaacatataattacatgtacaaatcaaaactaccttatgaaataaatagaaatattgcagttcatttctaatttaacctcttcaacttttaaaactatttacatttctttatgtcatttctagtcattttgatgcaaattgtaccatttatggattatcttcacaaatttttaagttggtgaaaactttttggtgggtagttaaaacttgaaatagaaatttactttaccaaaataaactaatgaaaagtaatcactccactccctataataagatttccaacgttcccactaagc

SEQ ID NO: 119 - DNA, 999 bp, Arabidopsis thaliana
  misc_feature (1)..(999): Ceres Promoter YP0022
tagttccattacaatttccaaatgatttgttacaaagctacaagattattcgaaataggatttcatccataagagagaatggtgtggtcgacgctacaatgttgatttattggttgtggtttgcatcttggggatgtcaaatcctaagtttcaagttcttgtaaaaacgttttcaggtttctttaatatattttaatattaatgtaaaaagaaaagatatagcttttgtacaaaaaaatttgtttaatcactatgtaggaggatgcgatcaaattcatggaatgatgtattattagcttttctatcctcactctaaaaacaatactatagtgagttaaataatttgatcatttcaatgtagattaaaattttattaaaagaagaaaaatttaaaagcctataacaaaataaaaaaggaggctcgaggtatgatgggtgtagcagaagagctggcaacagctatcgactgagtgattacgaactcagtactcagtgttctcagctcacacactctttttttgttctctttcttttggacagctttcattttctcttttcttttttctattttgtttcaaaattccatccatattaaaataggcctgatcatgagaataaaggaaatactaatgatgagtttctcaataatgcaataagatgcaattattatgagctatttactattgaaaatgagcaaataaatgtcaaaacacaatctggttaagttagagcaactccattgtataggattcatgtagtttctaagaaaacaaaatgtattaatattttacttttacatccaaaaaaccaacttatatgagtaatagaaacgatcctaatattaggaattttagagattttctctcatctgtttcttaacttttcaatatttttattttttaaaattgtatgagtttctactaagaaactactgctggagttggtcttagcttcccaatgcttctccacctatatatatgcatatctccttcttaaaac

SEQ ID NO: 120 - DNA, 999 bp, Arabidopsis thaliana
  misc_feature (1)..(999): Ceres Promoter YP0080
aagcggcaatttagtaagaagtactcaaagtatcatttaccaaaagtatatggttttgggaagagttgttagggatgtattctttctaaacagatgatatgacgatgttcttgaaaactaatgttaaagacggaatctctggcatcttcactcgggagatatattaaaccgttgattgtagttagccatgtacttagcttagtgcacaaataatctgctgcaagaaatctttttctattataatatctctcatttaaacattagaacatattgtttaacttgttcttctagaaataaaactgctaatttcttatggtaaactattttcctttagattgcacaatcgaactcgaaaatctagtggagactatgtgactatgtttatatatatgaaacctaaatcaaattatcccaataattgggagacacaaaagaaaaattacgaaagaaaacaggaaatcaaatcaaaagataaagagaaggtaaaaaaaggcaagaagcactaatgtttaatatttatagttttctccattaaagaaaaagcgatgatgtgtgttctcatcttttgtgaaagtatatatattgcttttgcttttctcaaaagcaaaagactcatccaacaagaacaaaaaaaaaaactaaagctcaatccaaaagacgaagaatgcattggatactacaacttctttttcacttttctttcaaatttacaattatgattttcacaatacagtttattcaaaaataaataaaaaaacgaggcatgaaaataatgattatcctcttcacttattaagccactcactataagcagagcaactccagaacatagtgagcccccaaaacattaaagcatgatgatgtctaatgatgatgatcttcttcgttccatttctctaaatttttgggatttctgcgaagacccttcttctctttctcttctctgaacttcaagattcgtgtcggacaaatttttgtttttatttttctgatgttaca

SEQ ID NO: 121 - DNA, 1000 bp, Arabidopsis thaliana
  misc_feature (1)..(1000): Ceres Promoter PT0565
caccaaatatagtgttatttcaatactaaaatggtgttatggttggagatgccctaaagataaacatgacgagacacgagatttattaatttcttgatcaaccataacttaataacttaatattaatttcacttaataatttccaattaagtgaatctttacttcaccaaaagttcctaacgaactcttattttctagcatcaatattaccatgaactagcatcaatactatcatgaaaaattcctacttcctatccaactcttaataacaatgctagtcttaacaatattcatcaaaaacttgatatagaccttctaacttagccacgactagtatcggtgaataccaaaattaatgtattcatgagaacttgagatttctctaatgtattcttgttactaaacaagtaacaacactcaagaaatatcatgatcaaatattttactcataaactccatatttcacattttgaaaattttaaacagcaaatcacattgaattttcgtggtaaaagtatttaaaattgaaaaatagcagctcctgatttcaatgtataaatttatctttatatggtttatgtctccaacttattttaaaaaagagagaaagagcacccaaaaggtgaccgtttgaaattcgaatttatttccgtttgaaattcgaattcaaaaaaagtaaaccgaaccgagtctcgttactgactgtcacacattgtttccctaaaagctaattaacccatacgtggcgtaatataacaggtcagtgatcaatactaaataacagacatacacctttaaaattcgtgcacgctccaaaacaaaatctacacttcaaaatcaacggtcacgatcattcctcaaatttcaaaaaattatttaacctcacttccttcgctttgtttttaaaacctctctctctttctctttctctttcgccattaaaactctgtttcctttttcagagattctcagagaagattcattttaccctaagaaaaaa

SEQ ID NO: 122 - DNA, 999 bp, Arabidopsis thaliana
  misc_feature (1)..(999): Ceres Promoter YP0015
ttgagccttattgttgttattgacttttagccaatagaaagagatggaaattcaataattatccacaaaattccaaatcattggtgtacaaaaagatctaaggctgttatattttcaaaaaagaaagaaaagaaatgcaacaaatatggattaaactgtggtttgtaaattgagctttgcatgaaaactttatcactatgatttcactactccatatttattgactaaagtggcactaatgaatttcttaatcatgaaatcttgtatcaaaaagtactaaaataaacatgacattggcaattaggaaaattctaaattagaaattagtaaaaatgaaaggtgaaagggaaagatgatgatatgaattggttggtgaccaggagaaatgtatcccgatttttgcagacactttcagtgtccccattcatataattatggcccacctcgttaagatttttcattcaccaccataacaagatctaagcttagatttcatgtaattaaacatataatatacttgccaatactatctaataaagtatacttaagcaaaaattattactctagtgtaaggcgatgaaatataagtttagttgaaaatttatgtcgatataacaaagtataatgaattaagaccttggttttcgattaacaaactaattaaacactagttttgcctaataaaaccgggaatcgtattcaaaaccgaacgacaaaacaagggacaagttgagagacaaaaccaaatcagcatctttcttccagaaatgtcatgaccacatgacgtcatcttgacccttcttcattgtgatatctgtggataaagcgcacgtgtttaattcacgaaccttcgtagtaacgaaaaatccacaactttcatattttttaattacccactaaactaaaacaaatttggaaaaacatgaaaaactttttctttttttccaggttcgtgaacctcgtaccctctatataaacctcttaaccaccttccacata